(19) United States
(12) Patent Application Publication
(10) Pub. No.: US 2025/0329417 A1
(43) Pub. Date: Oct. 23, 2025

(54) GUIDE DESIGN AND OFF-TARGET SEARCHES

(71) Applicant: CRISPR THERAPEUTICS AG, Zug (CH)

(72) Inventors: John Michael TOOMEY, South Boston, MA (US); Sarah J. SPENCER, Southt Boston, MA (US)

(21) Appl. No.: 18/860,591

(22) PCT Filed: Apr. 27, 2023
(86) PCT No.: PCT/IB2023/054329

§ 371 Date: Oct. 25, 2024

Related U.S. Application Data
(60) Provisional application No. 63/335,388, filed on Apr. 27, 2022.

(51) Int. Cl. G16B 30/10; C12N 9/22; C12N 15/11; C12N 15/90

(52) U.S. Cl. CPC G16B 30/10; C12N 9/226; C12N 15/11; C12N 15/907; C12N 2310/20

(57) ABSTRACT

Disclosed herein include systems, devices, and methods for determining a protospacer sequence. For each of protospacer sequences, homology strings of the protospacer sequence can be generated. Each of the homology strings can be mapped to a reference sequence sequence to determine a match of the homology string in the reference sequence. Matches of one or more of the homology strings of can be filtered based on a protospacer adjacent motif (PAM) space to determine one or more off-target sites of the protospacer sequence. A profile of each protospacer sequence can be determined using the off-target sites of the protospacer sequence. A protospacer sequence can be selected based on its profile. A guide comprising the selected protospacer sequence can be designed and used for gene editing.

### CROSS-REFERENCE TO RELATED APPLICATIONS

The present application is a U.S. national phase application under 35 U.S.C. § 371 of International Application No. PCT/IB2023/054329, filed on Apr. 27, 2023 and published as WO 2023/209614 A1 on Nov. 2, 2023, which claims the benefit of priority to U.S. Provisional Application No. 63/335,388, filed on Apr. 27, 2022. The content of each of these related applications is incorporated herein by reference in its entirety.

### REFERENCE TO SEQUENCE LISTING

The present application is being filed along with a Sequence Listing in electronic format. The Sequence Listing is provided as a file entitled 80EM-341700-US_SequenceListing, created Apr. 20, 2023, which is 14 kilobytes in size. The information in the electronic format of the Sequence Listing is incorporated herein by reference in its entirety.

### BACKGROUND

### Field

The present disclosure relates generally to the field of gene editing, and more particularly to guide design and off-target prediction.

### Description of the Related Art

Existing methods for guide designs and off-target prediction can be inefficient and slow, with many opportunities for user error. These methods have technical limitations in terms of search comprehensiveness. There is a need for improved methods for guide designs and off-target prediction that are efficient, fast, and comprehensive.

### SUMMARY

Disclosed herein include systems (or devices) for determining protospacer sequences in a sequence of interest. A sequence of interest can be a sequence for editing, such as gene editing. In some embodiments, a system (or device) for determining protospacer sequences in a sequence of interest comprises: non-transitory memory configured to store executable instructions. The non-transitory memory can be configured to store the reference sequence. The system can comprise: a processor (e.g., a hardware processor or a virtual processor, or two or more processors) in communication with the non-transitory memory. The processor can be programmed by the executable instructions to perform: receiving a sequence of interest. The processor can be programmed by the executable instructions to perform: determining a plurality of protospacer sequences (e.g., 1, 2, 3, 4, 5, 6, 7, 8, 9, 10, 15, 20, 30, 40, 50 or more protospacer sequences) in the sequence of interest. For example, the plurality of protospacer sequences can comprise some or all possible protospacer sequences in the sequence of interest. A protospacer sequence when present in a guide can be referred to as a spacer sequence (T(s) in the protospacer sequence would be U(s) in the spacer sequence). The processor can be programmed by the executable instructions to perform: generating a plurality of homology strings (e.g., 30, 40, 50, 60, 70, 80, 90, 100, 150, 200, 300, 400, 500, 750, 1000, or more, homology strings) of each of the plurality of protospacer sequences (or a plurality of homology strings of each of one or more of the protospacer sequences). The processor can be programmed by the executable instructions to perform: mapping (or aligning) each of the plurality of homology strings to a reference sequence (or a genome, or a sequence), such as a reference genome sequence, to determine a match (or at least one match, or one or more matches, such as 2, 3, 4, 5, 10, 15, 20, 30, 40, 50, 100, or more matches) of the homology string in the reference sequence. The match can be a perfect match (have zero mismatch) to (a subsequence of) the reference sequence. The match can have a perfect alignment to (a subsequence of) the reference sequence. The processor can be programmed by the executable instructions to perform: filtering (or removing) one or more of the matches of each of one or more homology strings of the plurality of homology strings, based on a protospacer adjacent motif (PAM) space, to determine one or more off-target sites of the protospacer sequence (e.g., 100, 1000, 2500, 5000, 7500, 10000, 25000, 50000, 75000, 100000, 250000, 500000, 750000, 1000000, or more off-target sites). The processor can be programmed by the executable instructions to perform: determining a protospacer sequence score of each of the plurality of protospacer sequences (or a protospacer sequence score of each of one or more protospacer sequences of the plurality of protospacer sequences) based on the off-target sites of the protospacer sequence. The processor can be programmed by the executable instructions to perform: determining a profile, of each of the plurality of protospacer sequences, comprising the protospacer sequence score of the protospacer sequence and based on the off-target sites of the protospacer sequence. The processor can be programmed by the executable instructions to perform: outputting each (or one or more, such as 1, 2, 3, 4, 5, 6, 7, 8, 9, 10, 15, 20, 30, 40, 50, or more) of the plurality of protospacer sequences and the profile of the protospacer sequence.

Disclosed herein include systems (or devices) for determining protospacer sequences in a sequence of interest (e.g., a sequence for editing). In some embodiments, a system (or a device) for determining protospacer sequences in a sequence of interest comprises: non-transitory memory configured to store executable instructions. The non-transitory memory can be configured to store the reference sequence. The system can comprise: a processor (e.g., a hardware processor or a virtual processor, or two or more processors) in communication with the non-transitory memory. The processor can be programmed by the executable instructions to perform: receiving a plurality of protospacer sequences (e.g., 1, 2, 3, 4, 5, 6, 7, 8, 9, 10, 15, 20, 30, 40, 50 or more protospacer sequences). For example, the plurality of protospacer sequences can comprise some or all possible protospacer sequences in the sequence of interest. A protospacer sequence when present in a guide can be referred to as a spacer sequence (T(s) in the protospacer sequence would be U(s) in the spacer sequence). The processor can be programmed by the executable instructions to perform: generating a plurality of homology strings (e.g., 30, 40, 50, 60, 70, 80, 90, 100, 150, 200, 300, 400, 500, 750, 1000, or more, homology strings) of each of the plurality of protospacer sequences (or a plurality of homology strings of each of one or more of the protospacer sequences). The processor can be programmed by the executable instructions to perform: mapping (or aligning) each of the plurality of homology strings to a reference sequence (or a genome, or a sequence), such as a reference genome sequence, to determine a match (or at least one match, or one or more matches, such as 2, 3, 4, 5, 10, 15, 20, 30, 40, 50, 100, or more matches) of the homology string in the reference sequence. The match can be a perfect match (have zero mismatch) to (a subsequence of) the reference sequence. The match can have a perfect alignment to (a subsequence of) the reference sequence. The processor can be programmed by the executable instructions to perform: filtering (or removing) one or more of the matches of each of one or more homology strings of the plurality of homology strings, based on a protospacer adjacent motif (PAM) space, to determine one or more off-target sites of the protospacer sequence (e.g., 100, 1000, 2500, 5000, 7500, 10000, 25000, 50000, 75000, 100000, 250000, 500000, 750000, 1000000, or more off-target sites). The processor can be programmed by the executable instructions to perform: determining a protospacer sequence score of each of the plurality of protospacer sequences (or a protospacer sequence score of each of one or more protospacer sequences of the plurality of protospacer sequences) based on the off-target sites of the protospacer sequence. The processor can be programmed by the executable instructions to perform: outputting each (or one or more, such as 1, 2, 3, 4, 5, 6, 7, 8, 9, 10, 15, 20, 30, 40, 50, or more) of the plurality of protospacer sequences and the protospacer sequence score of the protospacer sequence. In some embodiments, the processor is programmed by the executable instructions to perform: determining a profile, of each of the plurality of protospacer sequences, comprising the protospacer sequence score of the protospacer sequence and/or based on the off-target sites of the protospacer sequence. Outputting each of the plurality of protospacer sequences and the protospacer sequence score of the protospacer sequence can comprise: outputting each (or one or more, such as 1, 2, 3, 4, 5, 6, 7, 8, 9, 10, 15, 20, 30, 40, 50, or more) of the plurality of protospacer sequences and the profile of the protospacer sequence.

Disclosed herein include systems (or devices) for determining profiles of protospacer sequences. In some embodiments, a system (or a device) for determining profiles of protospacer sequences comprises: non-transitory memory configured to store executable instructions. The non-transitory memory can be configured to store the reference sequence. The system can comprise a processor (e.g., a hardware processor or a virtual processor, or two or more processors) in communication with the non-transitory memory. The processor can be programmed by the executable instructions to perform: receiving a plurality of protospacer sequences (e.g., 1, 2, 3, 4, 5, 6, 7, 8, 9, 10, 15, 20, 30, 40, 50 or more protospacer sequences). For example, the plurality of protospacer sequences can comprise some or all possible protospacer sequences in a sequence of interest. A protospacer sequence when present in a guide can be referred to as a spacer sequence (T(s) in the protospacer sequence would be U(s) in the spacer sequence). The processor can be programmed by the executable instructions to perform: for each of the plurality of protospacer sequences: generating a plurality of homology strings of the protospacer sequence (e.g., 30, 40, 50, 60, 70, 80, 90, 100, 150, 200, 300, 400, 500, 750, 1000, or more, homology strings). The processor can be programmed by the executable instructions to perform: mapping (or aligning) each of the plurality of homology strings to a reference sequence (or a genome, or a sequence), such as a reference genome sequence, to determine a match (or at least one match, or one or more matches, such as 2, 3, 4, 5, 10, 15, 20, 30, 40, 50, 100, or more matches) of the homology string in the reference sequence. The match can be a perfect match (have zero mismatch) to (a subsequence of) the reference sequence. The match can have a perfect alignment to (a subsequence of) the reference sequence. The processor can be programmed by the executable instructions to perform: filtering (or removing) one or more of the matches of homology strings of the plurality of homology strings, based on a protospacer adjacent motif (PAM) space, to determine one or more off-target sites of the protospacer sequence (e.g., 100, 1000, 2500, 5000, 7500, 10000, 25000, 50000, 75000, 100000, 250000, 500000, 750000, 1000000, or more off-target sites). The processor can be programmed by the executable instructions to perform: determining a profile of the protospacer sequence using the off-target sites of the protospacer sequence. In some embodiments, the profile of a protospacer sequence comprises a protospacer sequence score of the protospacer sequence. In some embodiments, the processor is programmed by the executable instructions to perform: outputting the profile of the protospacer sequence of each of one or more of the plurality of protospacer sequences.

In some embodiments, the plurality of protospacer sequences comprises protospacer sequences (e.g., some or all protospacer sequences) in the sequence of interest. In some embodiments, receiving the plurality of protospacer sequences comprises: receiving a sequence of interest. Receiving the plurality of protospacer sequences can comprise: determining the plurality of protospacer sequences in the sequence of interest.

In some embodiments, receiving the sequence of interest comprises: receiving the sequence of interest from a user interface (UI) element (e.g., a text field). In some embodiments, receiving the sequence of interest comprises: obtaining the sequence of interest from a file (e.g., a file in a storage device, e.g., a file in FASTA format or CSV format) and/or over a network (e.g., LAN, WAN, or Internet). In some embodiments, the sequence of interest comprises a gene, or a portion thereof. The sequence of interest can comprise an exon, or a portion thereof, of a gene and/or an intron, or a portion thereof, of a gene.

In some embodiments, the PAM space comprises a PAM sequence. The PAM sequence can be 2, 3, 4, 5, 6, or more nucleotides in length. The PAM space can comprise an on-target PAM sequence (e.g., NGG for SpCas9). Alternatively or additionally, the PAM space can comprise one or more off-target PAM sequences (e.g., NAG, NGA, NAA, NCG, NGC, NTG, and NGT for SpCas9). Alternatively or additionally, the PAM space can comprise a spacing (e.g., 1, 2, 3, 4, 5, 6, 7, 8, 9, or 10 nucleotides) between an PAM sequence and an associated protospacer sequence. Alternatively or additionally, the PAM space can comprise a spacing (e.g., 1, 2, 3, 4, 5, 6, 7, 8, 9, or 10 nucleotides) between an PAM sequence and a cleavage site in an associated protospacer sequence. Alternatively or additionally, the PAM space can comprise a relative positioning (e.g., 3′ or 5′) of an on-target PAM sequence and an associated protospacer sequence. In some embodiments, each of the plurality of protospacer sequences is associated with a PAM sequence (e.g., an on-target PAM sequence) in the reference sequence.

In some embodiments, determining the plurality of protospacer sequences in the sequence of interest comprises: determining the plurality of protospacer sequences in the sequence of interest based on the PAM space. Determining the plurality of protospacer sequences in the sequence of interest based on the PAM space can comprise: identifying an on-target PAM sequence in the sequence of interest. Determining the plurality of protospacer sequences in the sequence of interest based on the PAM space can comprise: identifying a protospacer sequence associated with the on-target PAM sequence in the sequence of interest using a protospacer length (e.g., 20 nucleotides in length, or 17, 18, 19, 20, 21, 22, 23, or 24 nucleotides in length), a spacing (e.g., 1, 2, 3, 4, 5, 6, 7, 8, 9, or 10 nucleotides) between an on-target PAM sequence and an associated protospacer sequence, and/or a relative positioning of an on-target PAM sequence and an associated protospacer sequence in the PAM space.

In some embodiments, a nucleic acid guided nuclease (or nucleic acid guided endonuclease or RNA-guided DNA endonuclease), or a portion thereof and/or a variant thereof (e.g., a nickase), is associated with the PAM space. The PAM space can be determined based on the specific nucleic acid guided nuclease, which can be selected. The nucleic acid guided nuclease can be associated with a protospacer length (e.g., 20 nucleotides in length, or 17, 18, 19, 20, 21, 22, 23, or 24 nucleotides in length). The nucleic acid guided nuclease can be a CRISPR-associated (Cas) nuclease of a species. The nucleic acid guided nuclease can be S. pyogenes Cas9 (SpCas9), S. aureus Cas9 (SaCas9), or S. lugdunensis Cas9 (slCas9). The nucleic acid guided nuclease can be a Class 1 Cas or Class 2 Cas. The nucleic acid guided nuclease can be a Cas of type I, II, III, IV, V, or VI. The nucleic acid guided nuclease can be Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, Cas9, Csn2, Cas4, Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5), C2c4, C2c8, C2c9, Cas13, Cas13a (C2c2), Cas13b, Cas13c, Cas13d, or Cas13x.1.

In some embodiments, the processor is programmed by the executable instructions to perform: receiving a selection of a nucleic acid guided nuclease (or nucleic acid guided endonuclease or RNA-guided DNA endonuclease), or a portion thereof and/or a variant thereof (e.g., a nickase). The processor can be programmed by the executable instructions to perform: obtaining (or selecting or retrieving) the PAM space associated with the nucleic acid guided nuclease. The processor can be programmed by the executable instructions to perform: receiving a selection of a reference sequence (e.g., a reference genome sequence of hg16, hg17, hg18, hg19, hg38, mm10, canFam4, chlSab2, macFas5, rheMac10, or rn6).

In some embodiments, each of the plurality of homology strings (e.g., 30, 40, 50, 60, 70, 80, 90, 100, 150, 200, 300, 400, 500, 750, 1000 or more) of a protospacer sequence comprises one or more mismatches (mm) (or zero, one, or more mismatches) relative to the protospacer sequence and/or one or more indels (or zero, one, or more indels) relative to the protospacer sequence. An indel can be referred to as a gap. An indel can be an insertion. An indel can be a deletion. The maximum number of mismatches can vary, such as 0, 1, 2, 3, 4, or 5 mismatches. The maximum number of indels can vary, such as 0, 1, 2, 3, 4, or 5 indels. In some embodiments, the maximum number of mismatches can be 5 when there is no indel. The maximum number of mismatches can be 2 when there is 1 indel (or at most 1 indel). The maximum number of mismatches can be 0 when there are 2 indels (or at most 2 indels). A homology string can be of a homology string type. A homology string type can comprise a combination of a number of mismatches and a number of indels, NmmXgap, where N can be for example 0, 1, 2, 3, 4, or 5, and X can be for example 0, 1, or 2, such as 0mm0gap, 1mm0gap, 2mm0gap, 3mm0gap, 4mm0gap, 5mm0gap, 0mm1gap, 1mm1gap, 2mm1gap, or 0mm2gap. In some embodiments, homology strings of the plurality of homology strings of a protospacer sequence with one mismatch, relative to the protospacer sequence, comprise all possible sequences with one mismatch at each position of the protospacer sequence. Homology strings of the plurality of homology strings of a protospacer sequence with two mismatches, relative to the protospacer sequence, can comprise all possible sequences with two mismatches relative to the protospacer sequence. Homology strings of the plurality of homology strings of a protospacer sequence with three mismatches, relative to the protospacer sequence, can comprise all possible sequences with three mismatches relative to the protospacer sequence. Homology strings of the plurality of homology strings of a protospacer sequence with four mismatches, relative to the protospacer sequence, can comprise all possible sequences with four mismatches relative to the protospacer sequence. Homology strings of the plurality of homology strings of a protospacer sequence with five mismatches, relative to the protospacer sequence, can comprise all possible sequences with five mismatches relative to the protospacer sequence. Homology strings of the plurality of homology strings of a protospacer sequence with one indel relative to the protospacer sequence can comprise all sequences with one indel at each position of the protospacer sequence. Homology strings of the plurality of homology strings of a protospacer sequence with two indels relative to the protospacer sequence can comprise all sequences with two indel relative to the protospacer sequence.

In some embodiments, the plurality of homology strings of a protospacer sequence comprises all (comprehensive or exhaustive) homology strings of the protospacer sequence of each of one or more homology string types, optionally wherein homology string type comprises a combination of a number of mismatches (e.g., 0, 1, 2, 3, 4, or 5 mismatches) and a number of indels (e.g., 0, 1, 2, 3, 4, or 5 indels). In some embodiments, the plurality of homology strings of a protospacer sequence comprises the protospacer sequence. Alternatively, the plurality of homology strings of a protospacer sequence does not comprise the protospacer sequence.

In some embodiments, a match of a homology string of a protospacer sequence comprises a perfect alignment (e.g., 0 mismatch) of the homology string to a position of the reference sequence. A corresponding off-target site of the protospacer sequence can comprise an alignment of the off-target site to the position of the reference sequence that is not a perfect alignment.

In some embodiments, filtering one or more of the matches of each of the one or more homology strings comprises: removing from the matches of each of the one or more homology strings one or more of the matches of the homology string. The one or more off-target sites of the protospacer sequence can comprise the remaining matches of the homology string. The remaining matches of the plurality of homology strings can be the one or more off-target sites In some embodiments, filtering one or more of the matches of the one or more homology strings comprises: filtering a match of a homology string, based on an absence of a PAM sequence (e.g., an on-target PAM sequence) being associated with the match in the reference sequence (e.g., the match does not have an associated PAM sequence in the genome), to determine one or more off-target sites of the protospacer sequence. In some embodiments, filtering one or more of the matches of the one or more homology strings comprises: filtering a match of a homology string, based on an absence of any on-target PAM sequence and/or any off-target PAM sequence being associated with the match in the reference sequence, to determine one or more off-target sites of the protospacer sequence. The one or more off-target sites of the protospacer sequence can be comprehensive (e.g., 100%) of the off-target sites of the protospacer sequence. The one or more off-target sites can comprise at least 99% (or 95%, 96%, 97%, 98%, 99%, 99.9%, 99.99%, or more) of all possible off-target sites of the protospacer sequence.

In some embodiments, the processor is programmed by the executable instructions to perform: filtering the one or more off-target sites of the protospacer sequence using low complexity region (LCR) filtering to generated one or more filtered off-target sites. Determining the protospacer sequence score of each of the plurality of protospacer sequences can comprise: determining the protospacer sequence score of each of the plurality of protospacer sequences based on the filtered off-target sites of the protospacer sequence. Determining the profile of each of the plurality of protospacer sequences can comprise: determining the profile, of each of the plurality of protospacer sequences, comprising the protospacer sequence score of the protospacer sequence and based on the filtered off-target sites of the protospacer sequence.

In some embodiments, determining the protospacer sequence score of each of the plurality of protospacer sequences comprises: determining an off-target site score for each of the one or more off-target sites of the protospacer sequence. Determining the protospacer sequence score of each of the plurality of protospacer sequences can comprise: determining a protospacer sequence score of each of the plurality of protospacer sequences using the off-target site scores of the one or more off-target sites of the protospacer sequence.

In some embodiments, the protospacer sequence score is based on a number of the off-target sites. The protospacer sequence score can be based on the distribution of mismatches of the off-target sites. The protospacer sequence score can be based on the distance of an off-target site to the closest annotated exon. The protospacer sequence score can reflect a strength of interaction between a guide comprising the protospacer sequence and a target of the guide. The protospacer sequence score can comprise an off-target score, a CCTop score and/or a CFD score.

In some embodiments, the processor is programmed by the executable instructions to perform: consolidating two of the off-target sites of a protospacer sequence that overlap to generate consolidated off-target sites of the protospacer sequence. The processor can be programmed by the executable instructions to perform: consolidating overlapping off-target sites of the off-target sites of a protospacer sequence to generate consolidated off-target sites of the protospacer sequence. In some embodiments, determining the protospacer sequence score comprises: determining a protospacer sequence score of each of the plurality of protospacer sequences based on the consolidated off-target sites of the protospacer sequence.

In some embodiments, the profile of a protospacer sequence comprises an off-target profile of the protospacer sequence. In some embodiments, the profile of a protospacer sequence comprises a summary of the off-target sites of the protospacer sequence. The summary of the off-target sties of the protospacer sequence can comprise a number of one or more matches of the protospacer sequence in the reference sequence. The summary of the off-target sties of the protospacer sequence can comprise a number of off-target sites of the protospacer sequence for each of one or more homology string types.

In some embodiments, the processor is programmed by the executable instructions to perform: ranking and/or sorting the plurality of protospacer sequences based on the protospacer sequence scores and/or the profiles. Outputting each of the plurality of protospacer sequences and the profile of the protospacer sequence can comprise: outputting each of the plurality of protospacer sequences and the profile of the protospacer sequence comprises based on the ranking and/or sorting.

In some embodiments, outputting each of the protospacer sequences and the profile of the protospacer sequence comprises: outputting each of the plurality of protospacer sequences and the profile of the protospacer sequence to one or more files. Outputting each of the protospacer sequences and the profile of the protospacer sequence can comprise: generating a user interface (UI) comprises one or more UI elements representing each of the plurality of protospacer sequences and the profile of the protospacer sequence.

Disclosed herein include methods for determining a profile of a protospacer sequence. In some embodiments, a method for determining a profile of a protospacer sequence can be under control of a processor (e.g., a hardware processor or a virtual processor, or two or more processors). The method can comprise: receiving a sequence of interest. The method can comprise: determining a protospacer sequence in the sequence of interest. A protospacer sequence when present in a guide can be referred to as a spacer sequence (T(s) in the protospacer sequence would be U(s) in the spacer sequence). The method can comprise: generating homology strings of the protospacer sequence (e.g., 30, 40, 50, 60, 70, 80, 90, 100, 150, 200, 300, 400, 500, 750, 1000, or more, homology strings). The method can comprise: mapping (or aligning) the homology strings to a reference sequence (or a genome, or a sequence), such as a reference genome sequence, to determine matches (e.g., 100, 1000, 2500, 5000, 7500, 10000, 25000, 50000, 75000, 100000, 250000, 500000, 750000, 1000000, or more, matches) of the homology strings in the reference sequence. The match can be a perfect match (have zero mismatch) to (a subsequence of) the reference sequence. The match can have a perfect alignment to (a subsequence of) the reference sequence. The method can comprise: filtering (or removing) one or more (e.g., 10, 20, 30, 40, 50, 100, 500, 1000, or more) of the matches of the homology strings, based on a protospacer adjacent motif (PAM) space, to determine one or more off-target sites of the protospacer sequence (e.g., 100, 1000, 2500, 5000, 7500, 10000, 25000, 50000, 75000, 100000, 250000, 500000, 750000, 1000000, or more off-target sites). The method can comprise: determining a profile of the protospacer sequence using the off-target sites of the protospacer sequence.

Disclosed herein include methods for determining a profile of a protospacer sequence. In some embodiments, a method for determining a profile of a protospacer sequence comprises: receiving a protospacer sequence in a sequence of interest. A protospacer sequence when present in a guide can be referred to as a spacer sequence (T(s) in the protospacer sequence would be U(s) in the spacer sequence). The method can comprise: generating a plurality of homology strings of the protospacer sequence. The method can comprise: mapping (or aligning) each of one or more of the plurality of homology strings to a reference sequence or a genome, or a sequence), such as a reference genome sequence, to determine a match (or at least one match, or one or more matches, such as 2, 3, 4, 5, 10, 15, 20, 30, 40, 50, 100, or more matches) of the homology string in the reference sequence. The match can be a perfect match (have zero mismatch) to (a subsequence of) the reference sequence. The match can have a perfect alignment to (a subsequence of) the reference sequence. The method can comprise: filtering (removing) one or more of the matches of homology strings of the plurality of homology strings, based on a protospacer adjacent motif (PAM) space, to determine one or more off-target sites of the protospacer sequence. The method can comprise: determining a profile of the protospacer sequence using the off-target sites of the protospacer sequence. In some embodiments, the method comprises: outputting the protospacer sequence and the profile of the protospacer sequence.

Disclosed herein include methods of editing a sequence. In some embodiments, a method for editing a sequence comprises: obtaining a guide comprising a protospacer sequence of a sequence of interest. The protospacer sequence can be selected from a plurality of protospacer sequences (e.g., 1, 2, 3, 4, 5, 6, 7, 8, 9, 10, 15, 20, 30, 40, 50 or more protospacer sequences) of the sequence of interest. For example, the plurality of protospacer sequences can comprise some or all possible protospacer sequences in a sequence of interest. The protospacer sequence can be selected from a plurality of protospacer sequences of the sequence of interest by: for each of the plurality of protospacer sequences of the sequence of interest: generating a plurality of homology strings of the protospacer sequence (e.g., 30, 40, 50, 60, 70, 80, 90, 100, 150, 200, 300, 400, 500, 750, 1000, or more, homology strings). For example, the plurality of protospacer sequences can comprise some or all possible protospacer sequences in the sequence of interest. A protospacer sequence when present in a guide can be referred to as a spacer sequence (T(s) in the protospacer sequence would be U(s) in the spacer sequence). The protospacer sequence can be selected from a plurality of protospacer sequences of the sequence of interest by: mapping (or aligning) each of the plurality of homology strings to a reference sequence (or a genome, or a sequence), such as a reference genome sequence, to determine a match (or at least one match, or one or more matches, such as 2, 3, 4, 5, 10, 15, 20, 30, 40, 50, 100, or more matches) of the homology string in the reference sequence. The match can be a perfect match (have zero mismatch) to (a subsequence of) the reference sequence. The match can have a perfect alignment to (a subsequence of) the reference sequence. The protospacer sequence can be selected from a plurality of protospacer sequences of the sequence of interest by: filtering (or removing) one or more of the matches of homology strings of the plurality of homology strings, based on a protospacer adjacent motif (PAM) space, to determine one or more off-target sites of the protospacer sequence. The protospacer sequence can be selected from a plurality of protospacer sequences of the sequence of interest by: determining a profile of the protospacer sequence using the off-target sites of the protospacer sequence. The protospacer sequence can be selected from a plurality of protospacer sequences of the sequence of interest by: selecting the protospacer sequence from the plurality of protospacer sequences of the sequence of interest based on the profile of the protospacer sequence selected (or based on the profile of each of one or more of the plurality of protospacer sequences). The method can comprise: editing a sequence in a nucleic acid using the guide and a nucleic acid guided nuclease (or nucleic acid guided endonuclease or RNA-guided DNA endonuclease), or a portion thereof and/or a variant thereof (e.g., a nickase).

Disclosed herein include methods of for generating a guide for editing a sequence. In some embodiments, a method for generating a guide for editing a sequence comprises: receiving a plurality of protospacer sequences (e.g., 1, 2, 3, 4, 5, 6, 7, 8, 9, 10, 15, 20, 30, 40, 50 or more protospacer sequences). For example, the plurality of protospacer sequences can comprise some or all possible protospacer sequences in the sequence of interest. A protospacer sequence when present in a guide can be referred to as a spacer sequence (T(s) in the protospacer sequence would be U(s) in the spacer sequence). The method can comprise, for each of the plurality of protospacer sequences: generating a plurality of homology strings of the protospacer sequence (e.g., 30, 40, 50, 60, 70, 80, 90, 100, 150, 200, 300, 400, 500, 750, 1000, or more, homology strings). The method can comprise: mapping each of the plurality of homology strings to a reference sequence to determine a match (or at least one match, or one or more matches, such as 2, 3, 4, 5, 10, 15, 20, 30, 40, 50, 100, or more matches) of the homology string in the reference sequence. The match can be a perfect match (have zero mismatch) to (a subsequence of) the reference sequence. The match can have a perfect alignment to (a subsequence of) the reference sequence. The method can comprise: filtering (or removing) one or more of the matches of homology strings of the plurality of homology strings, based on a protospacer adjacent motif (PAM) space, to determine one or more off-target sites of the protospacer sequence (e.g., 100, 1000, 2500, 5000, 7500, 10000, 25000, 50000, 75000, 100000, 250000, 500000, 750000, 1000000, or more off-target sites). The method can comprise: determining a profile of the protospacer sequence using the off-target sites of the protospacer sequence. The method can comprise: obtaining a guide comprising a protospacer sequence of the plurality of protospacer sequences. The guide can be selected based on the profiles of protospacer sequences of the plurality of protospacer sequences (or based on the profile of each of the plurality of protospacer sequences). The method can comprise: selecting the protospacer sequence based on the profiles of protospacer sequences of the plurality of protospacer sequences.

In some embodiments, the protospacer sequence of the guide has the best profile (e.g., the best protospacer sequence score, or the protospacer sequence with fewest predicted off-target sites and/or least impactful off-target sites) among profiles of protospacer sequences of the plurality of protospacer sequences.

In some embodiments, obtaining the guide comprises: designing the guide. In some embodiments, the guide comprises a guide ribonucleic acid (gRNA). The guide can comprise a single guide RNA (sgRNA). The sgRNA can comprise a prime editing guide RNA (pegRNA). In some embodiments, the method comprises: determining an empirical profile (e.g., editing efficiency, off-target profile) of the guide.

In some embodiments, the method comprises: editing a sequence in a nucleic acid (e.g., deoxyribonucleic acid or DNA) using the guide and a nucleic acid guided nuclease (or nucleic acid guided endonuclease or RNA-guided DNA endonuclease), or a portion thereof and/or a variant thereof (e.g., a nickase). The editing can be base editing or prime editing. The nucleic acid can be in a cell. The cell can be in a subject, e.g., a mammal, such as a human. The nucleic acid guided nuclease can be a CRISPR-associated (Cas) nuclease of a species. The nucleic acid guided nuclease can be S. pyogenes Cas9 (SpCas9), S. aureus Cas9 (SaCas9), or S. lugdunensis Cas9 (slCas9). The nucleic acid guided nuclease can be a Class 1 Cas or Class 2 Cas. The nucleic acid guided nuclease can be a Cas of type I, II, III, IV, V, or VI. The nucleic acid guided nuclease can be Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, Cas9, Csn2, Cas4, Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5), C2c4, C2c8, C2c9, Cas13, Cas13a (C2c2), Cas13b, Cas13c, Cas13d, or Cas13x.1.

In some embodiments, wherein the plurality of protospacer sequences comprises protospacer sequences in a sequence of interest (e.g., all possible protospacer sequences in a sequence of interest). In some embodiments, the profile of a protospacer sequence comprises a protospacer sequence score of the protospacer sequence. Determining the profile of the protospacer sequence can comprise: determining a protospacer sequence score of the protospacer sequence using the off-target sites of the protospacer sequence. In some embodiments, the method comprises: outputting the profile of the protospacer sequence of each of one or more of the plurality of protospacer sequences. In some embodiments, receiving the plurality of protospacer sequences comprises: receiving a sequence of interest. Receiving the plurality of protospacer sequences can comprise: determining the plurality of protospacer sequences in the sequence of interest.

In some embodiments, receiving the sequence of interest comprises: receiving the sequence of interest from a user interface (UI) element (e.g., a text field). In some embodiments, receiving the sequence of interest comprises: obtaining the sequence of interest from a file (e.g., a file in a storage device, e.g., a file in FASTA format or CSV format) and/or over a network (e.g., LAN, WAN, or Internet). In some embodiments, the sequence of interest comprises a gene, or a portion thereof. The sequence of interest can comprise an exon, or a portion thereof, of a gene and/or an intron, or a portion thereof, of a gene.

In some embodiments, the PAM space comprises a PAM sequence. The PAM sequence can be 2, 3, 4, 5, 6, or more nucleotides in length. The PAM space can comprise an on-target PAM sequence (e.g., NGG for SpCas9). Alternatively or additionally, the PAM space can comprise one or more off-target PAM sequences (e.g., NAG, NGA, NAA, NCG, NGC, NTG, and NGT for SpCas9). Alternatively or additionally, the PAM space can comprise a spacing (e.g., 1, 2, 3, 4, 5, 6, 7, 8, 9, or 10 nucleotides) between an PAM sequence and an associated protospacer sequence. Alternatively or additionally, the PAM space can comprise a spacing (e.g., 1, 2, 3, 4, 5, 6, 7, 8, 9, or 10 nucleotides) between an PAM sequence and a cleavage site in an associated protospacer sequence. Alternatively or additionally, the PAM space can comprise a relative positioning (e.g., 3′ or 5′) of an on-target PAM sequence and an associated protospacer sequence. In some embodiments, each of the plurality of protospacer sequences is associated with a PAM sequence (e.g., an on-target PAM sequence) in the reference sequence.

In some embodiments, determining the plurality of protospacer sequences in the sequence of interest comprises: determining the plurality of protospacer sequences in the sequence of interest based on the PAM space. Determining the plurality of protospacer sequences in the sequence of interest based on the PAM space can comprise: identifying an on-target PAM sequence in the sequence of interest. Determining the plurality of protospacer sequences in the sequence of interest based on the PAM space can comprise: identifying a protospacer sequence associated with the on-target PAM sequence in the sequence of interest using a protospacer length (e.g., 20 nucleotides in length, or 17, 18, 19, 20, 21, 22, 23, or 24 nucleotides in length), a spacing between an on-target PAM sequence and an associated protospacer sequence (e.g., 1, 2, 3, 4, 5, 6, 7, 8, 9, or 10 nucleotides), and/or a relative positioning (e.g., 3′ or 5′) of an on-target PAM sequence and an associated protospacer sequence in the PAM space.

In some embodiments, a nucleic acid guided nuclease (or nucleic acid guided endonuclease or RNA-guided DNA endonuclease), or a portion thereof and/or a variant thereof (e.g., a nickase), is associated with the PAM space. The PAM space can be determined based on the specific nucleic acid guided nuclease, which can be selected. The nucleic acid guided nuclease can be associated with a protospacer length (e.g., 20 nucleotides in length, or 17, 18, 19, 20, 21, 22, 23, or 24 nucleotides in length). The nucleic acid guided nuclease can be a CRISPR-associated (Cas) nuclease of a species. The nucleic acid guided nuclease can be S. pyogenes Cas9 (SpCas9), S. aureus Cas9 (SaCas9), or S. lugdunensis Cas9 (slCas9). The nucleic acid guided nuclease can be a Class 1 Cas or Class 2 Cas. The nucleic acid guided nuclease can be a Cas of type I, II, III, IV, V, or VI. The nucleic acid guided nuclease can be Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, Cas9, Csn2, Cas4, Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5), C2c4, C2c8, C2c9, Cas13, Cas13a (C2c2), Cas13b, Cas13c, Cas13d, or Cas13x.1.

In some embodiments, the method comprises: receiving a selection of a nucleic acid guided nuclease (or nucleic acid guided endonuclease or RNA-guided DNA endonuclease), or a portion thereof and/or a variant thereof (e.g., a nickase). The method can comprise: obtaining (or selecting or retrieving) the PAM space associated with the nucleic acid guided nuclease. The method can comprise: receiving a selection of a reference sequence (e.g., a reference genome sequence of hg16, hg17, hg18, hg19, hg38, mm10, canFam4, chlSab2, macFas5, rheMac10, or rn6).

In some embodiments, each of the plurality of homology strings (e.g., 30, 40, 50, 60, 70, 80, 90, 100, 150, 200, 300, 400, 500, 750, 1000 or more) of a protospacer sequence comprises one or more mismatches (mm) (or zero, one, or more mismatches) relative to the protospacer sequence and/or one or more indels (or zero, one, or more indels) relative to the protospacer sequence. An indel can be referred to as a gap. An indel can be an insertion. An indel can be a deletion. The maximum number of mismatches can vary, such as 0, 1, 2, 3, 4, or 5 mismatches. The maximum number of indels can vary, such as 0, 1, 2, 3, 4, or 5 indels. In some embodiments, the maximum number of mismatches can be 5 when there is no indel. The maximum number of mismatches can be 2 when there is 1 indel (or at most 1 indel). The maximum number of mismatches can be 0 when there are 2 indels (or at most 2 indels). A homology string can be of a homology string type. A homology string type can comprise a combination of a number of mismatches and a number of indels, NmmXgap, where N can be for example 0, 1, 2, 3, 4, or 5, and X can be for example 0, 1, or 2, such as 0mm0gap, 1mm0gap, 2mm0gap, 3mm0gap, 4mm0gap, 5mm0gap, 0mm1gap, 1mm1gap, 2mm1gap, or 0mm2gap. In some embodiments, homology strings of the plurality of homology strings of a protospacer sequence with one mismatch, relative to the protospacer sequence, comprise all possible sequences with one mismatch at each position of the protospacer sequence. Homology strings of the plurality of homology strings of a protospacer sequence with two mismatches, relative to the protospacer sequence, can comprise all possible sequences with two mismatches relative to the protospacer sequence. Homology strings of the plurality of homology strings of a protospacer sequence with three mismatches, relative to the protospacer sequence, can comprise all possible sequences with three mismatches relative to the protospacer sequence. Homology strings of the plurality of homology strings of a protospacer sequence with four mismatches, relative to the protospacer sequence, can comprise all possible sequences with four mismatches relative to the protospacer sequence. Homology strings of the plurality of homology strings of a protospacer sequence with five mismatches, relative to the protospacer sequence, can comprise all possible sequences with five mismatches relative to the protospacer sequence. Homology strings of the plurality of homology strings of a protospacer sequence with one indel relative to the protospacer sequence can comprise all sequences with one indel at each position of the protospacer sequence. Homology strings of the plurality of homology strings of a protospacer sequence with two indels relative to the protospacer sequence can comprise all sequences with two indel relative to the protospacer sequence.

In some embodiments, the plurality of homology strings of a protospacer sequence comprises all (comprehensive or exhaustive) homology strings of the protospacer sequence of each of one or more homology string types, optionally wherein homology string type comprises a combination of a number of mismatches (e.g., 0, 1, 2, 3, 4, or 5 mismatches) and a number of indels (e.g., 0, 1, 2, 3, 4, or 5 indels). In some embodiments, the plurality of homology strings of a protospacer sequence comprises the protospacer sequence. Alternatively, the plurality of homology strings of a protospacer sequence does not comprise the protospacer sequence.

In some embodiments, a match of a homology string of a protospacer sequence comprises a perfect alignment (e.g., 0 mismatch) of the homology string to a position of the reference sequence. A corresponding off-target site of the protospacer sequence can comprise an alignment of the off-target site to the position of the reference sequence that is not a perfect alignment.

In some embodiments, filtering one or more of the matches of the homology strings comprises: removing from the matches of the homology strings of the plurality of homology string one or more of the matches of the homology strings. The one or more off-target sites of the protospacer sequence can comprise the remaining matches of the plurality of homology strings. The remaining matches of the plurality of homology strings can be the one or more off-target sites. In some embodiments, filtering one or more of the matches of the homology strings comprises: filtering a match of a homology string, based on an absence of a PAM sequence (e.g., an on-target PAM sequence) being associated with the match in the reference sequence (e.g., the match does not have an associated PAM sequence in the genome), to determine one or more off-target sites of the protospacer sequence. In some embodiments, filtering one or more of the matches of the homology strings comprises: filtering a match of a homology string, based on an absence of any on-target PAM sequence and/or any off-target PAM sequence being associated with the match in the reference sequence, to determine one or more off-target sites of the protospacer sequence. The one or more off-target sites of the protospacer sequence can be comprehensive (e.g., 100%) of the off-target sites of the protospacer sequence. The one or more off-target sites can comprise at least 99% (or 95%, 96%, 97%, 98%, 99%, 99.9%, 99.99%, or more) of all possible off-target sites of the protospacer sequence.

In some embodiments, the method comprises: filtering the one or more off-target sites of the protospacer sequence using low complexity region (LCR) filtering to generated one or more filtered off-target sites. Determining the protospacer sequence score of the protospacer sequence can comprise: determining the protospacer sequence score of the protospacer sequence using the filtered off-target sites of the protospacer sequence. Determining the profile of the protospacer sequence can comprise: determining the profile of the protospacer sequence using the filtered off-target sites of the protospacer sequence.

In some embodiments, determining the protospacer sequence score of the protospacer sequence comprises: determining an off-target site score for each of the one or more off-target sites of the protospacer sequence. Determining the protospacer sequence score of the protospacer sequence can comprise: determining the protospacer sequence score of the protospacer sequence using the off-target site scores of the one or more off-target sites of the protospacer sequence.

In some embodiments, the protospacer sequence score is based on a number of the off-target sites. The protospacer sequence score can be based on the distribution of mismatches of the off-target sites. The protospacer sequence score can be based on the distance of an off-target site to the closest annotated exon. The protospacer sequence score can reflect a strength of interaction between a guide comprising the protospacer sequence and a target of the guide. The protospacer sequence score can comprise an off-target score, a CCTop score and/or a CFD score.

In some embodiments, the method comprises: consolidating two of the off-target sites of a protospacer sequence that overlap to generate consolidated off-target sites of the protospacer sequence. The method comprises: consolidating overlapping off-target sites of the off-target sites of a protospacer sequence to generate consolidated off-target sites of the protospacer sequence. In some embodiments, determining the protospacer sequence score comprises: determining a protospacer sequence score of each of the plurality of protospacer sequences based on the consolidated off-target sites of the protospacer sequence.

In some embodiments, the profile of a protospacer sequence comprises an off-target profile of the protospacer sequence. In some embodiments, the profile of a protospacer sequence comprises a summary of the off-target sites of the protospacer sequence. The summary of the off-target sties of the protospacer sequence can comprise a number of one or more matches of the protospacer sequence in the reference sequence. The summary of the off-target sties of the protospacer sequence can comprise a number of off-target sites of the protospacer sequence for each of one or more homology string types.

In some embodiments, the method comprises: ranking and/or sorting the plurality of protospacer sequences based on the protospacer sequence scores and/or the profiles. Outputting each of the plurality of protospacer sequences and the profile of the protospacer sequence can comprise: outputting each of the plurality of protospacer sequences and the profile of the protospacer sequence comprises based on the ranking and/or sorting.

In some embodiments, outputting each of the protospacer sequences and the profile of the protospacer sequence comprises: outputting the profile of the protospacer sequence of each of one or more of the plurality of protospacer sequences and the profile of the protospacer sequence to one or more files. In some embodiments, outputting each of the protospacer sequences and the profile of the protospacer sequence comprises: generating a user interface (UI) comprises one or more UI elements representing, or a report comprising, the profile of the protospacer sequence of each of one or more of the plurality of protospacer sequences and the profile of the protospacer sequence.

Also disclosed herein include a non-transitory computer-readable medium storing executable instructions, when executed by a system (e.g., a computing system) or a device, causes the system to perform any method or one or more steps of a method disclosed herein.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1 displays a non-limiting exemplary cartoon of CRISPR-Cas9 mediated DNA editing.

FIG. 2 displays a exemplary on-target and off-target sites of a guide spacer sequence.

FIG. 3 displays examples of how previous methods of identifying off-target sites can miss off-target sequences during guide design.

FIG. 4 shows a non-limiting exemplary flow diagram of the AVOLANCHE strategy disclosed herein.

FIG. 5 displays an exemplary flow diagram of where AVOLANCHE can be deployed in a CRISPR-Cas9 experimental design.

FIG. 6 depicts an exemplary use case for the methods disclosed herein (e.g., to disrupt an exon of a gene).

FIG. 7A-FIG. 7F depict non-limiting exemplary use of the AVOLANCHE tool disclosed herein.

FIG. 8-FIG. 9B depict exemplary outputs of the AVOLANCHE tool disclosed herein.

FIG. 10A-FIG. 10B depict non-limiting exemplary data showing that the AVOLANCHE tool can find more sites (FIG. 10A) in less time (FIG. 10B) than previous workflow.

FIG. 11 displays non-limiting exemplary data showing that the disclosed methods can find additional off-target sites as compared to previous tools.

FIG. 12 shows that AVOLANCHE does not miss sites that exist in the genome.

FIG. 13 displays a non-limiting exemplary block diagram of AVOLANCHE workflow.

FIG. 14 displays a non-limiting exemplary chart of homology string generation.

FIG. 15A-FIG. 15C show how deletions, mismatches, and insertions are calculated using formulas for calculating expected sequences for sequences with maximum 1 gap.

FIG. 16 displays a non-limiting exemplary flow diagram for a brute-force approach used to validate the AVOLANCHE methods disclosed herein.

FIG. 17 displays flowcharts for comparing standard workflows and the disclosed AVOLANCHE method.

FIG. 18 displays number of sites found by AVOLANCHE as compared to standard workflow.

FIG. 19 displays non-limiting exemplary data showing that after removing low-complexity regions (LCRs), AVOLANCHE still identified more sites as compared to a standard workflow.

FIG. 20 depicts non-limiting exemplary data showing that AVOLANCHE found that do not overlap any site found by a standard workflow.

FIG. 21 displays data showing that standard workflow (e.g., CCTop and CRISPOR) missed ungapped sites.

FIG. 22 displays exemplary mismatched and/or gapped sites with non-NRG PAMs missed by standard workflow (e.g., COSMID). “-”, gap; “*/.”, mismatch.

FIG. 23 displays data showing that standard workflow (e.g., CCTop AND CRISPOR) missed some 3 mm sites.

FIG. 24 displays non-limiting exemplary data showing standard workflow (e.g., CCTop and CRISPOR) missed sites with 2 mismatches and no gaps.

FIG. 25 displays non-limiting exemplary data showing that after LCR-filtering, the AVOLANCHE method disclosed herein found sites that do not overlap with any site found using a standard workflow.

FIG. 26 displays a graph showing that the disclosed AVOLANCHE method can find more sites as compared to a standard workflow (e.g., prior to consolidation).

FIG. 27 displays a non-limiting exemplary chart showing that AVOLANCHE can find sites with many possible alignments, which can be consolidated.

FIG. 28A-FIG. 28B display Venn diagrams showing data related to alternative chromosome sites are not 100% redundant in two different AVOLANCHE-generated data sets.

FIG. 29A-FIG. 29B show a non-limiting exemplary single web-app approach of AVOLANCHE.

FIG. 30 displays a non-limiting exemplary multi web-app approach of AVOLANCHE.

FIG. 31 shows a non-limiting exemplary flowchart for AVOLANCHE to LCR filter integration.

FIG. 32 is a flow diagram showing an exemplary method of determining profiles (e.g., off-target profiles) of protospacer sequences. A protospacer sequence can be selected based on its profile and used to design a guide for gene editing.

FIG. 33 is a block diagram of an illustrative computing system configured to implement guide design and off-target searches.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Overview

Existing methods for guide designs and off-target prediction can be inefficient and slow, with many opportunities for user error. These methods have technical limitations in terms of search comprehensiveness. There is a need for improved methods for guide designs and off-target prediction that are efficient, fast, and comprehensive.

Disclosed herein include systems (or devices) for determining protospacer sequences in a sequence of interest. A sequence of interest can be a sequence for editing, such as gene editing. A system or a device can perform any method (or a portion thereof) of the present disclosure. In some embodiments, a system (or device) for determining protospacer sequences in a sequence of interest comprises: non-transitory memory configured to store executable instructions. The non-transitory memory can be configured to store the reference sequence. The system can comprise: a processor (e.g., a hardware processor or a virtual processor, or two or more processors) in communication with the non-transitory memory. The processor can be programmed by the executable instructions to perform: receiving a sequence of interest. The processor can be programmed by the executable instructions to perform: determining a plurality of protospacer sequences (e.g., 1, 2, 3, 4, 5, 6, 7, 8, 9, 10, 15, 20, 30, 40, 50 or more protospacer sequences) in the sequence of interest. For example, the plurality of protospacer sequences can comprise some or all possible protospacer sequences in the sequence of interest. A protospacer sequence when present in a guide can be referred to as a spacer sequence (T(s) in the protospacer sequence would be U(s) in the spacer sequence). The processor can be programmed by the executable instructions to perform: generating a plurality of homology strings (e.g., 30, 40, 50, 60, 70, 80, 90, 100, 150, 200, 300, 400, 500, 750, 1000, or more, homology strings) of each of the plurality of protospacer sequences (or a plurality of homology strings of each of one or more of the protospacer sequences). The processor can be programmed by the executable instructions to perform: mapping (or aligning) each of the plurality of homology strings to a reference sequence (or a genome, or a sequence), such as a reference genome sequence, to determine a match (or at least one match, or one or more matches, such as 2, 3, 4, 5, 10, 15, 20, 30, 40, 50, 100, or more matches) of the homology string in the reference sequence. The match can be a perfect match (have zero mismatch) to (a subsequence of) the reference sequence. The match can have a perfect alignment to (a subsequence of) the reference sequence. The processor can be programmed by the executable instructions to perform: filtering (or removing) one or more of the matches of each of one or more homology strings of the plurality of homology strings, based on a protospacer adjacent motif (PAM) space, to determine one or more off-target sites of the protospacer sequence (e.g., 100, 1000, 2500, 5000, 7500, 10000, 25000, 50000, 75000, 100000, 250000, 500000, 750000, 1000000, or more off-target sites). The processor can be programmed by the executable instructions to perform: determining a protospacer sequence score of each of the plurality of protospacer sequences (or a protospacer sequence score of each of one or more protospacer sequences of the plurality of protospacer sequences) based on the off-target sites of the protospacer sequence. The processor can be programmed by the executable instructions to perform: determining a profile, of each of the plurality of protospacer sequences, comprising the protospacer sequence score of the protospacer sequence and based on the off-target sites of the protospacer sequence. The processor can be programmed by the executable instructions to perform: outputting each (or one or more, such as 1, 2, 3, 4, 5, 6, 7, 8, 9, 10, 15, 20, 30, 40, 50, or more) of the plurality of protospacer sequences and the profile of the protospacer sequence.

Disclosed herein include systems (or devices) for determining protospacer sequences in a sequence of interest (e.g., a sequence for editing). In some embodiments, a system (or a device) for determining protospacer sequences in a sequence of interest comprises: non-transitory memory configured to store executable instructions. The non-transitory memory can be configured to store the reference sequence. The system can comprise: a processor (e.g., a hardware processor or a virtual processor, or two or more processors) in communication with the non-transitory memory. The processor can be programmed by the executable instructions to perform: receiving a plurality of protospacer sequences (e.g., 1, 2, 3, 4, 5, 6, 7, 8, 9, 10, 15, 20, 30, 40, 50 or more protospacer sequences). For example, the plurality of protospacer sequences can comprise some or all possible protospacer sequences in the sequence of interest. A protospacer sequence when present in a guide can be referred to as a spacer sequence (T(s) in the protospacer sequence would be U(s) in the spacer sequence). The processor can be programmed by the executable instructions to perform: generating a plurality of homology strings (e.g., 30, 40, 50, 60, 70, 80, 90, 100, 150, 200, 300, 400, 500, 750, 1000, or more, homology strings) of each of the plurality of protospacer sequences (or a plurality of homology strings of each of one or more of the protospacer sequences). The processor can be programmed by the executable instructions to perform: mapping (or aligning) each of the plurality of homology strings to a reference sequence (or a genome, or a sequence), such as a reference genome sequence, to determine a match (or at least one match, or one or more matches, such as 2, 3, 4, 5, 10, 15, 20, 30, 40, 50, 100, or more matches) of the homology string in the reference sequence. The match can be a perfect match (have zero mismatch) to (a subsequence of) the reference sequence. The match can have a perfect alignment to (a subsequence of) the reference sequence. The processor can be programmed by the executable instructions to perform: filtering (or removing) one or more of the matches of each of one or more homology strings of the plurality of homology strings, based on a protospacer adjacent motif (PAM) space, to determine one or more off-target sites of the protospacer sequence (e.g., 100, 1000, 2500, 5000, 7500, 10000, 25000, 50000, 75000, 100000, 250000, 500000, 750000, 1000000, or more off-target sites). The processor can be programmed by the executable instructions to perform: determining a protospacer sequence score of each of the plurality of protospacer sequences (or a protospacer sequence score of each of one or more protospacer sequences of the plurality of protospacer sequences) based on the off-target sites of the protospacer sequence. The processor can be programmed by the executable instructions to perform: outputting each (or one or more, such as 1, 2, 3, 4, 5, 6, 7, 8, 9, 10, 15, 20, 30, 40, 50, or more) of the plurality of protospacer sequences and the protospacer sequence score of the protospacer sequence. In some embodiments, the processor is programmed by the executable instructions to perform: determining a profile, of each of the plurality of protospacer sequences, comprising the protospacer sequence score of the protospacer sequence and/or based on the off-target sites of the protospacer sequence. Outputting each of the plurality of protospacer sequences and the protospacer sequence score of the protospacer sequence can comprise: outputting each (or one or more, such as 1, 2, 3, 4, 5, 6, 7, 8, 9, 10, 15, 20, 30, 40, 50, or more) of the plurality of protospacer sequences and the profile of the protospacer sequence.

Disclosed herein include systems (or devices) for determining profiles of protospacer sequences. In some embodiments, a system (or a device) for determining profiles of protospacer sequences comprises: non-transitory memory configured to store executable instructions. The non-transitory memory can be configured to store the reference sequence. The system can comprise a processor (e.g., a hardware processor or a virtual processor, or two or more processors) in communication with the non-transitory memory. The processor can be programmed by the executable instructions to perform: receiving a plurality of protospacer sequences (e.g., 1, 2, 3, 4, 5, 6, 7, 8, 9, 10, 15, 20, 30, 40, 50 or more protospacer sequences). For example, the plurality of protospacer sequences can comprise some or all possible protospacer sequences in a sequence of interest. A protospacer sequence when present in a guide can be referred to as a spacer sequence (T(s) in the protospacer sequence would be U(s) in the spacer sequence). The processor can be programmed by the executable instructions to perform: for each of the plurality of protospacer sequences: generating a plurality of homology strings of the protospacer sequence (e.g., 30, 40, 50, 60, 70, 80, 90, 100, 150, 200, 300, 400, 500, 750, 1000, or more, homology strings). The processor can be programmed by the executable instructions to perform: mapping (or aligning) each of the plurality of homology strings to a reference sequence (or a genome, or a sequence), such as a reference genome sequence, to determine a match (or at least one match, or one or more matches, such as 2, 3, 4, 5, 10, 15, 20, 30, 40, 50, 100, or more matches) of the homology string in the reference sequence. The match can be a perfect match (have zero mismatch) to (a subsequence of) the reference sequence. The match can have a perfect alignment to (a subsequence of) the reference sequence. The processor can be programmed by the executable instructions to perform: filtering (or removing) one or more of the matches of homology strings of the plurality of homology strings, based on a protospacer adjacent motif (PAM) space, to determine one or more off-target sites of the protospacer sequence (e.g., 100, 1000, 2500, 5000, 7500, 10000, 25000, 50000, 75000, 100000, 250000, 500000, 750000, 1000000, or more off-target sites). The processor can be programmed by the executable instructions to perform: determining a profile of the protospacer sequence using the off-target sites of the protospacer sequence. In some embodiments, the profile of a protospacer sequence comprises a protospacer sequence score of the protospacer sequence. In some embodiments, the processor is programmed by the executable instructions to perform: outputting the profile of the protospacer sequence of each of one or more of the plurality of protospacer sequences.

Disclosed herein include systems (or devices) for performing method (or a portion thereof) of the present disclosure. In some embodiments, a system (or a device) comprises: non-transitory memory configured to store executable instructions. The non-transitory memory can be configured to store the reference sequence. The system can comprise a processor (e.g., a hardware processor or a virtual processor, or two or more processors) in communication with the non-transitory memory. The processor can be programmed by the executable instructions to perform: any method (or a portion thereof) of the present disclosure. A processor of a system or a device can perform any method (or a portion thereof) of the present disclosure.

Disclosed herein include methods for determining a profile of a protospacer sequence. In some embodiments, a method for determining a profile of a protospacer sequence can be under control of a processor (e.g., a hardware processor or a virtual processor, or two or more processors). The method can comprise: receiving a sequence of interest. The method can comprise: determining a protospacer sequence in the sequence of interest. A protospacer sequence when present in a guide can be referred to as a spacer sequence. The method can comprise: generating homology strings of the protospacer sequence (e.g., 30, 40, 50, 60, 70, 80, 90, 100, 150, 200, 300, 400, 500, 750, 1000, or more, homology strings). The method can comprise: mapping (or aligning) the homology strings to a reference sequence (or a genome, or a sequence), such as a reference genome sequence, to determine matches (e.g., 100, 1000, 2500, 5000, 7500, 10000, 25000, 50000, 75000, 100000, 250000, 500000, 750000, 1000000, or more, matches) of the homology strings in the reference sequence. The match can be a perfect match (have zero mismatch) to (a subsequence of) the reference sequence. The match can have a perfect alignment to (a subsequence of) the reference sequence. The method can comprise: filtering (or removing) one or more (e.g., 10, 20, 30, 40, 50, 100, 500, 1000, or more) of the matches of the homology strings, based on a protospacer adjacent motif (PAM) space, to determine one or more off-target sites of the protospacer sequence (e.g., 100, 1000, 2500, 5000, 7500, 10000, 25000, 50000, 75000, 100000, 250000, 500000, 750000, 1000000, or more off-target sites). The method can comprise: determining a profile of the protospacer sequence using the off-target sites of the protospacer sequence.

Disclosed herein include methods for determining a profile of a protospacer sequence. In some embodiments, a method for determining a profile of a protospacer sequence comprises: receiving a protospacer sequence in a sequence of interest. A protospacer sequence when present in a guide can be referred to as a spacer sequence. The method can comprise: generating a plurality of homology strings of the protospacer sequence. The method can comprise: mapping (or aligning) each of one or more of the plurality of homology strings to a reference sequence or a genome, or a sequence), such as a reference genome sequence, to determine a match (or at least one match, or one or more matches, such as 2, 3, 4, 5, 10, 15, 20, 30, 40, 50, 100, or more matches) of the homology string in the reference sequence. The match can be a perfect match (have zero mismatch) to (a subsequence of) the reference sequence. The match can have a perfect alignment to (a subsequence of) the reference sequence. The method can comprise: filtering (removing) one or more of the matches of homology strings of the plurality of homology strings, based on a protospacer adjacent motif (PAM) space, to determine one or more off-target sites of the protospacer sequence. The method can comprise: determining a profile of the protospacer sequence using the off-target sites of the protospacer sequence. In some embodiments, the method comprises: outputting the protospacer sequence and the profile of the protospacer sequence.

Disclosed herein include methods of editing a sequence. In some embodiments, a method for editing a sequence comprises: obtaining a guide comprising a protospacer sequence of a sequence of interest. The protospacer sequence can be selected from a plurality of protospacer sequences (e.g., 1, 2, 3, 4, 5, 6, 7, 8, 9, 10, 15, 20, 30, 40, 50 or more protospacer sequences) of the sequence of interest. For example, the plurality of protospacer sequences can comprise some or all possible protospacer sequences in a sequence of interest. The protospacer sequence can be selected from a plurality of protospacer sequences of the sequence of interest by: for each of the plurality of protospacer sequences of the sequence of interest: generating a plurality of homology strings of the protospacer sequence (e.g., 30, 40, 50, 60, 70, 80, 90, 100, 150, 200, 300, 400, 500, 750, 1000, or more, homology strings). For example, the plurality of protospacer sequences can comprise some or all possible protospacer sequences in the sequence of interest. A protospacer sequence when present in a guide can be referred to as a spacer sequence (T(s) in the protospacer sequence would be U(s) in the spacer sequence). The protospacer sequence can be selected from a plurality of protospacer sequences of the sequence of interest by: mapping (or aligning) each of the plurality of homology strings to a reference sequence (or a genome, or a sequence), such as a reference genome sequence, to determine a match (or at least one match, or one or more matches, such as 2, 3, 4, 5, 10, 15, 20, 30, 40, 50, 100, or more matches) of the homology string in the reference sequence. The match can be a perfect match (have zero mismatch) to (a subsequence of) the reference sequence. The match can have a perfect alignment to (a subsequence of) the reference sequence. The protospacer sequence can be selected from a plurality of protospacer sequences of the sequence of interest by: filtering (or removing) one or more of the matches of homology strings of the plurality of homology strings, based on a protospacer adjacent motif (PAM) space, to determine one or more off-target sites of the protospacer sequence. The protospacer sequence can be selected from a plurality of protospacer sequences of the sequence of interest by: determining a profile of the protospacer sequence using the off-target sites of the protospacer sequence. The protospacer sequence can be selected from a plurality of protospacer sequences of the sequence of interest by: selecting the protospacer sequence from the plurality of protospacer sequences of the sequence of interest based on the profile of the protospacer sequence selected (or based on the profile of each of one or more of the plurality of protospacer sequences). The method can comprise: editing a sequence in a nucleic acid using the guide and a nucleic acid guided nuclease.

Disclosed herein include methods of for generating a guide for editing a sequence. In some embodiments, a method for generating a guide for editing a sequence comprises: receiving a plurality of protospacer sequences (e.g., 1, 2, 3, 4, 5, 6, 7, 8, 9, 10, 15, 20, 30, 40, 50 or more protospacer sequences). For example, the plurality of protospacer sequences can comprise some or all possible protospacer sequences in the sequence of interest. A protospacer sequence when present in a guide can be referred to as a spacer sequence (T(s) in the protospacer sequence would be U(s) in the spacer sequence). The method can comprise, for each of the plurality of protospacer sequences: generating a plurality of homology strings of the protospacer sequence (e.g., 30, 40, 50, 60, 70, 80, 90, 100, 150, 200, 300, 400, 500, 750, 1000, or more, homology strings). The method can comprise: mapping each of the plurality of homology strings to a reference sequence to determine a match (or at least one match, or one or more matches, such as 2, 3, 4, 5, 10, 15, 20, 30, 40, 50, 100, or more matches) of the homology string in the reference sequence. The match can be a perfect match (have zero mismatch) to (a subsequence of) the reference sequence. The match can have a perfect alignment to (a subsequence of) the reference sequence. The method can comprise: filtering (or removing) one or more of the matches of homology strings of the plurality of homology strings, based on a protospacer adjacent motif (PAM) space, to determine one or more off-target sites of the protospacer sequence (e.g., 100, 1000, 2500, 5000, 7500, 10000, 25000, 50000, 75000, 100000, 250000, 500000, 750000, 1000000, or more off-target sites). The method can comprise: determining a profile of the protospacer sequence using the off-target sites of the protospacer sequence. The method can comprise: obtaining a guide comprising a protospacer sequence of the plurality of protospacer sequences. The guide can be selected based on the profiles of protospacer sequences of the plurality of protospacer sequences (or based on the profile of each of the plurality of protospacer sequences). The method can comprise: selecting the protospacer sequence based on the profiles of protospacer sequences of the plurality of protospacer sequences.

Also disclosed herein include a non-transitory computer-readable medium storing executable instructions, when executed by a system (e.g., a computing system) or a device, causes the system to perform any method or one or more steps of a method disclosed herein.

A method (or a system or device) for determining protospacer sequences and their profiles (or off-target prediction/determination and/or guide design) can be referred to herein as AVOLANCHE. A protospacer sequence can be selected based on its profile and a guide comprising the protospacer sequence can be designed and used for gene editing. A protospacer sequence when present in a guide can be referred to as a spacer sequence (T(s) in the protospacer sequence would be U(s) in the spacer sequence). A method (or a system or device) for determining protospacer sequences and their profiles can be efficient and fast. A method (or a system or device) for determining protospacer sequences and their profiles can be comprehensive (or exhaustive). A method (or a system or device) for determining protospacer sequences and their profiles can have search comprehensiveness. A method (or a system or device) for determining protospacer sequences and their profiles can be a method that is not a brute force method. A method (or a system or device) for determining protospacer sequences and their profiles can avoid user error. A method (or a system or device) for determining protospacer sequences and their profiles can be easily updated for new or additional nucleic acid guided nuclease (e.g., Cas proteins) and/or new genomes (or genome sequences). A method (or a system or device) for determining protospacer sequences and their profiles can be used for both mismatch gap prediction. A method (or a system or device) for determining protospacer sequences and their profiles can have a scalable infrastructure. A method (or a system or device) for determining protospacer sequences and their profiles can allow for modular extensions and/or allow new features.

### Guide Design and Off-Target Searches

Embodiments of guide design and off-target searches of the present disclosure can have one or more of the following capabilities as described below.

### Expanded Homology Search Space

Previous tools are limited in terms of the off-target homology space that can be searched: (i) CCTop/Guido: Up to 5mm0gap, no gapped search available; (ii) COSMID: Up to 3mm0gap, 2mm1gap; (iii) CRISPOR: Up to 4mm0gap, no gapped search available. In order to be as comprehensive as possible, results had to be combined across different tools to come up with the final predicted off-target site list for a given guide. AVOLANCHE has added an option to search for off-target sites with up to 2gaps relative to the gRNA sequence that was not previously available with the other tools. With AVOLANCHE, a 5mm0gap, 2mm1gap search can be performed with a single tool. The tool has successfully run up to a 4mm0gap, 3mm1gap, 2mm2gaps homology space, though could go higher in some embodiments. Running with higher homology searches than was possible with the previous three tools allows expanded gapped off-target searches.

Gapped searches were previously limited to advanced GACT users, since COSMID was too slow for most users to use on a regular basis. Most users were previously using CCTop/Guido for the initial gRNA design, which was not comprehensive. This required that GACT then ran those same guides through COSMID and CRISPOR at a later date. Now all searches can be performed with a single tool. Running with higher homology searches also enables new capabilities, such as performing more expanded searches for human variants that could result in editing activity. AVOLANCHE is also faster, allowing users to iterate through guide design and off-target searches faster.

### PAM Flexibility

AVOLANCHE treats input PAMs as a motif, rather than as part of the sequence to be searched for mismatches and gaps, like other tools do. This make specification of PAM sequences easier and enables users to iterate through different lists of PAMs at on-targets and off-targets more readily.

### 5′ PAM Guides

AVOLANCHE has the ability to find guides with 5′ PAM sequences and perform corresponding off-target searches. Currently, the only major Cas ortholog known to have a 5′ PAM sequence is Cpf1/Cas12a.

### Site Consolidation

AVOLANCHE performs an exhaustive search, so sometimes it finds multiple alignments between the guide and a given off-target site within the homology search space. In order to prevent AVOLANCHE from outputting too many sites at the same location, consolidation of alignments can be performed. In some embodiments, AVOLANCHE consolidates two alignments together into the same output site if their PAM start coordinates are within 2*(max number of gaps) of one another. In some embodiments, AVOLANCHE may be modified to consolidate two alignments together into the same site in several possible ways: (1) their protospacer sequences overlap one another; (2) their protospacer+PAM sequences overlap one another; (3) their PAM sequences overlap one another.

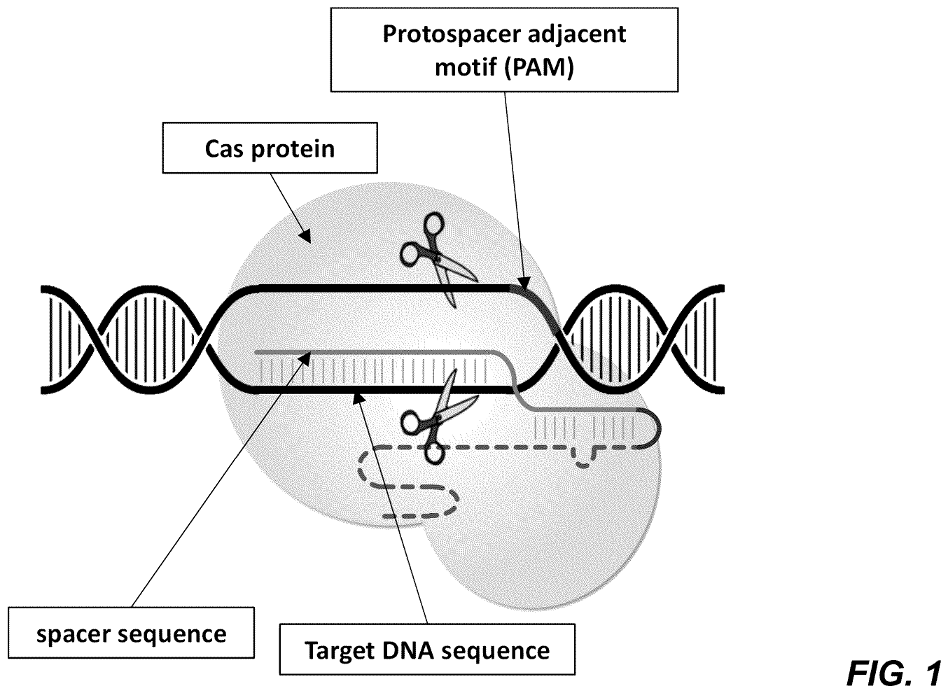

CRISPR/Cas9 editing of a DNA sequence involves Cas9+gRNA binding to a target site (FIG. 1). Sometimes binding and editing can occur at unintended sites, termed off-target editing. Non-limiting examples of factors that can contribute to off-target editing include: mismatches and gaps between, e.g., spacer and target are more tolerated when they occur distant from the protospacer adjacent motif (PAM); some Cas variants are more specific due to protein structure and PAM length; some 20 bp sequences are more unique in the genome (without being bound by any particular theory, there can be less opportunity for cleavage); off-target cleavage can be more likely in open chromatin.

In some embodiments, CRISPR off-target editing has consequences for drug safety and efficacy. In some embodiments, edits can occur in tumor suppressors, oncogenes, or oncogenic regions. In some embodiments, competing off-target sites can reduce on-target cleavage efficiency. In some embodiments, reducing off-target editing can advantageously reduce possibilities for large deletions and translocations. In some embodiments, off-target sites may create unanticipated phenotypic changes in cells.

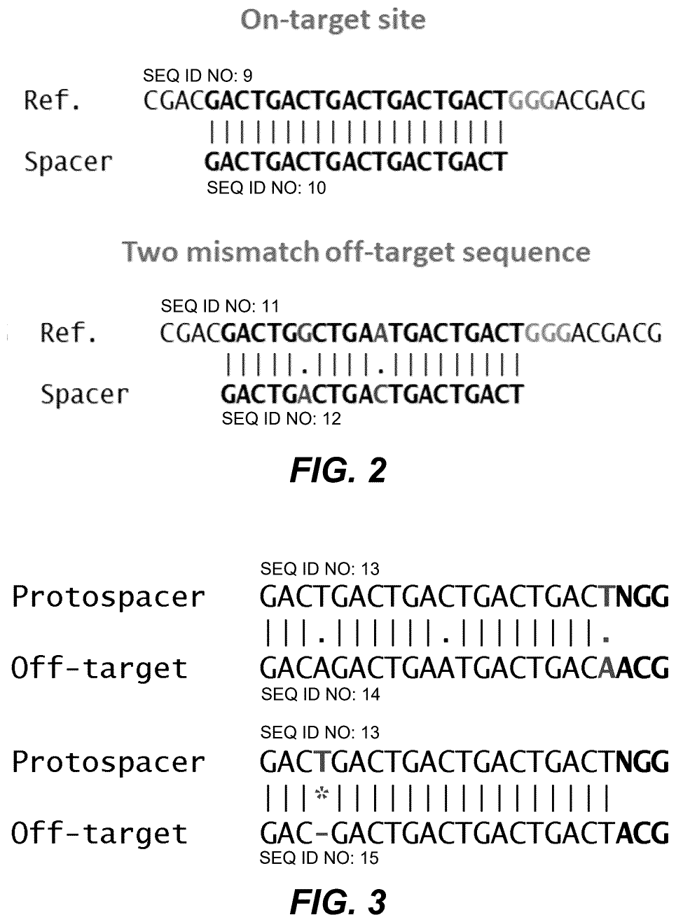

Off-targets can generally be defined based on homology to the guide, meaning they can contain mismatches (mm) and/or gaps relative to the guide spacer sequence (FIG. 2). Using computational bioinformatics tools and a guide sequence, one can predict where sites with homology to a guide exist in a genome even before ordering the guides or performing any experiments. Previous workflows (e.g., Guido) can miss off-target sites during guide design. For example, Guido can't find sites with mismatches in the first two bases adjacent to the PAM and/or sites with gaps (FIG. 3).

Multiple tools can be used for experimentally assessing off-targets for guides of interest (e.g., Guido, as well as CRISPOR, COSMID and low-complexity region filter). In some embodiments of a standard workflow, three off-target search algorithms are used to nominate sites-Guido, COSMID, and CRISPOR-all with different inputs, outputs, and capabilities. One additional tool can be used to merge results from those three and filter by an input list of desired PAMs. Maintaining four different tools to perform one task is difficult. Current tools as described above are inefficient and slow, with many opportunities for user error. It can be hard to update four tools to find targets for new Cas proteins in new genomes. No single tool can be used for mismatch and gap prediction with a scalable infrastructure, and each tool has technical limitations in terms of search comprehensiveness. Using four different tools does not allow for modular extensions, preventing new features, and not all tools are available to bench scientists looking to design new guide RNAs. A solution to the above problems in the art are provided by the methods disclosed herein. A Variant-aware Off-target Location Algorithm for Nominating CRISPR Homology-based Events (AVOLANCHE) is a new tool as a one-stop-shop for CRISPR guide design and off-target prediction needs.

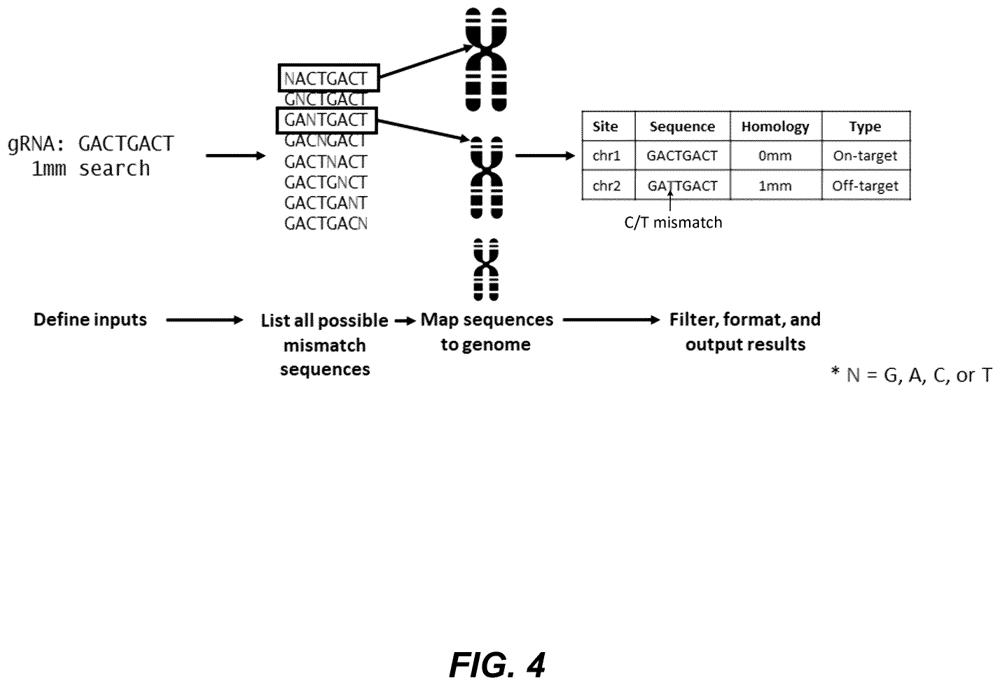

AVOLANCHE solves many of the issues described above. As shown in FIG. 4, AVOLANCHE uses an exhaustive approach for its search strategy. A number of features available through AVOLANCHE make it an improvement for guide design and off-target prediction. AVOLANCHE uses a PAM-agnostic approach that simplifies PAM input requirements. Implementation of AVOLANCHE in a more modern programming language with a simpler architecture makes it easier to add new features. Searches of equivalent homology spaces run more quickly than older tools. A comprehensive search enables higher off-target homology spaces. Addition of new genomes is faster with a more modular input/output structure. AVOLANCHE has been validated for a range of different use cases (Table 1).

TABLE 1
EXEMPLARY AVOLANCHE USE CASES

| Use case | Impact |
|---|---|
| Guides with accurate off-target | Can easily perform guide and off-target searches for |
| scoring | novel guide design |
| Predicting putative off-targets | Runs off-target searches in homology spaces with more |
| accounting for human genetic | mismatches and gaps than available with old tools |
| variation | |
| Estimating off-target in rapidly | Much more quickly (and independently) perform cross- |
| changing model organism genomes | species guide and off-target searches |
| Guides for Cas orthologs with | Can perform guide and off-target searches for novel |
| flexible design criteria | PAM sequences |

Described below is a non-limiting example of using the AVOLANCHE Web Platform.

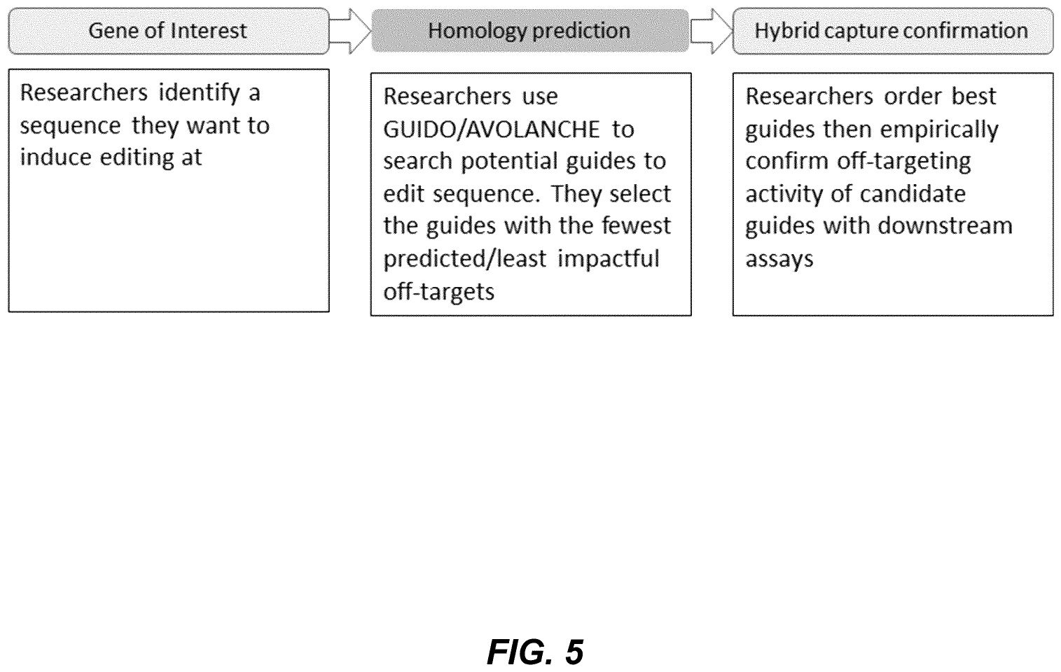

Previous workflows (e.g., GUIDO) have several disadvantages, including, but not limited to: the GUIDO algorithm can't search off-targets that have indels or for certain PAMs; GUIDO is unstable and not always available. The method disclosed herein has several advantages. In some embodiments, AVOLANCHE is advantageously comprehensive in examining off-targets with indels and atypical PAMs. FIG. 5 displays an exemplary flowchart showing where AVOLANCHE can fit in the research workflow.

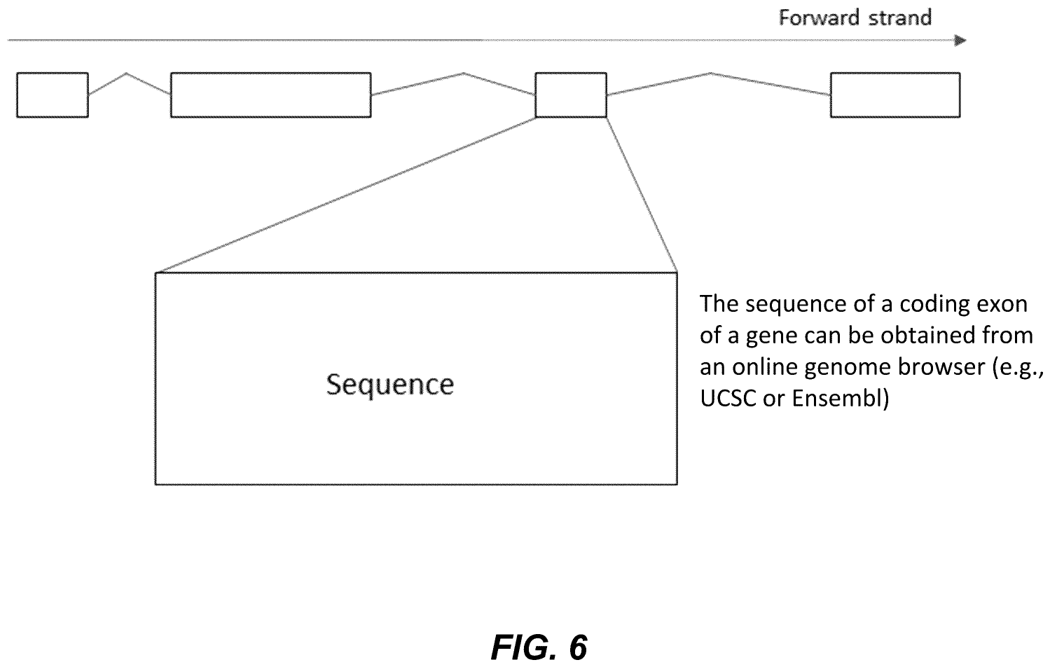

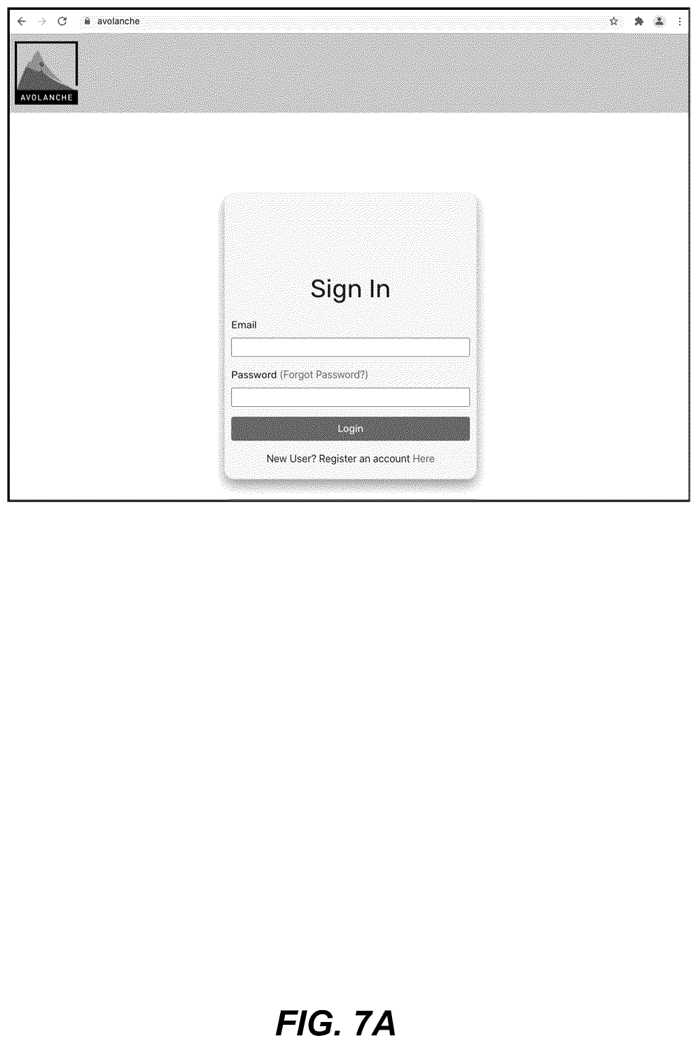

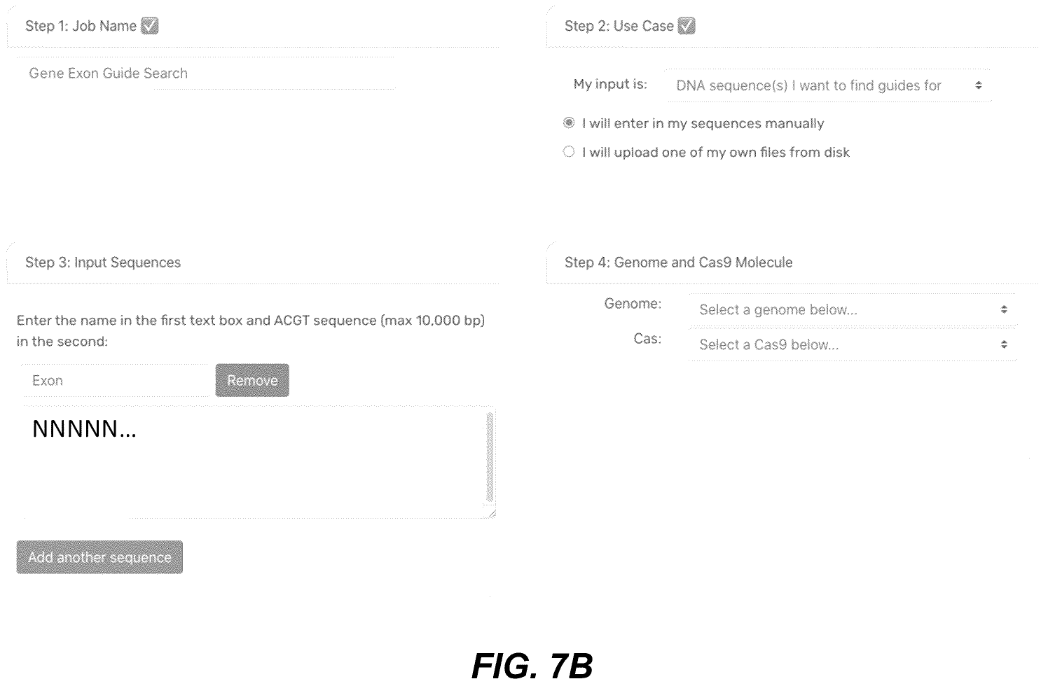

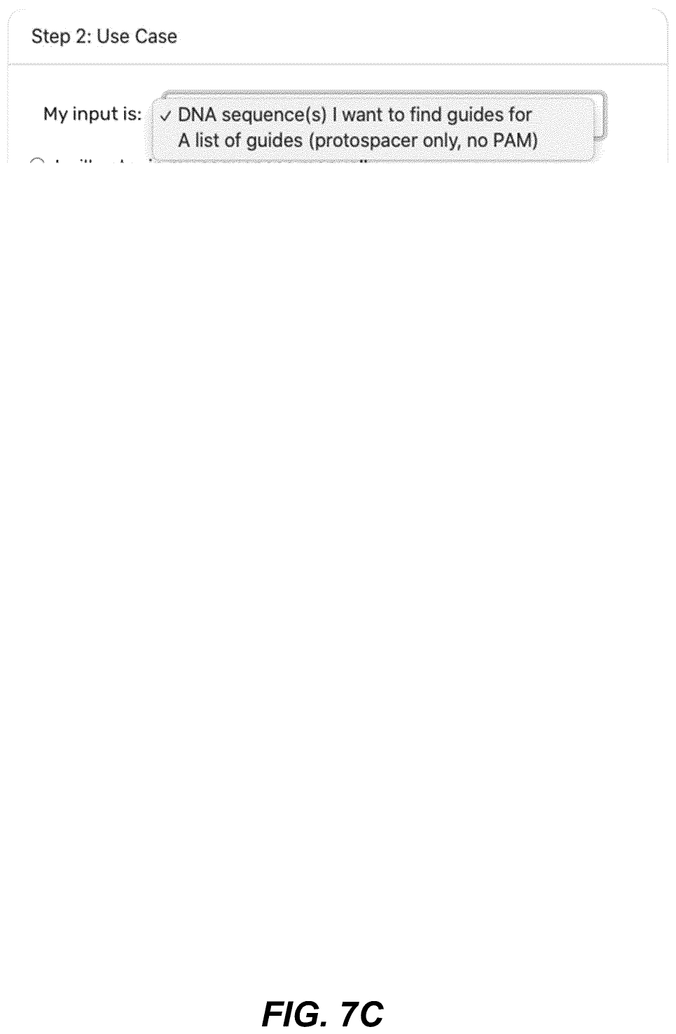

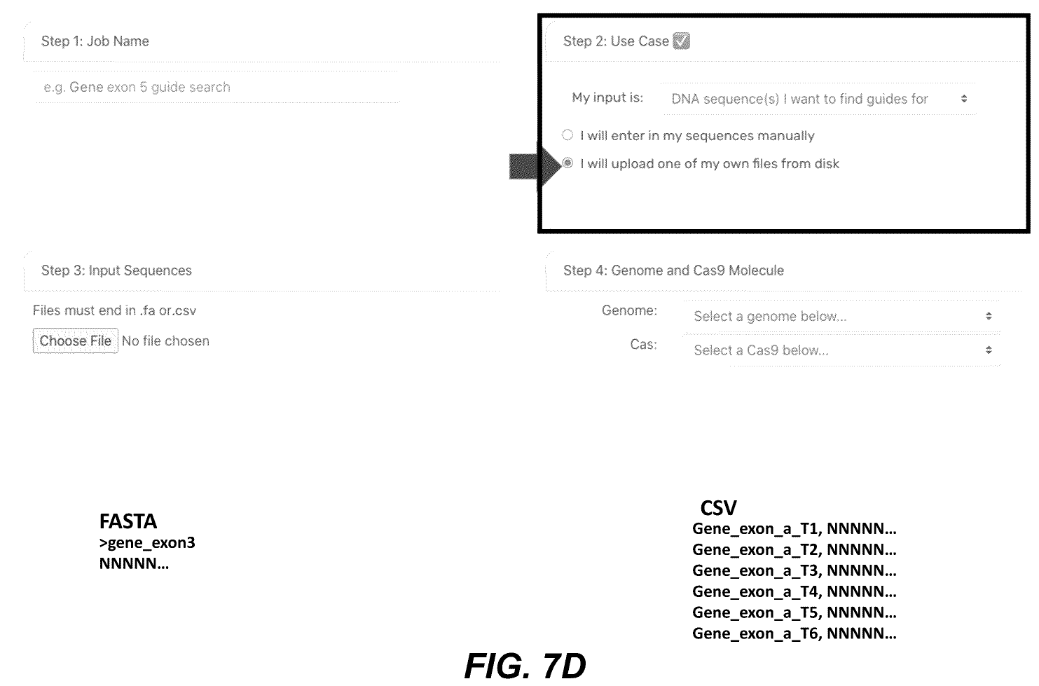

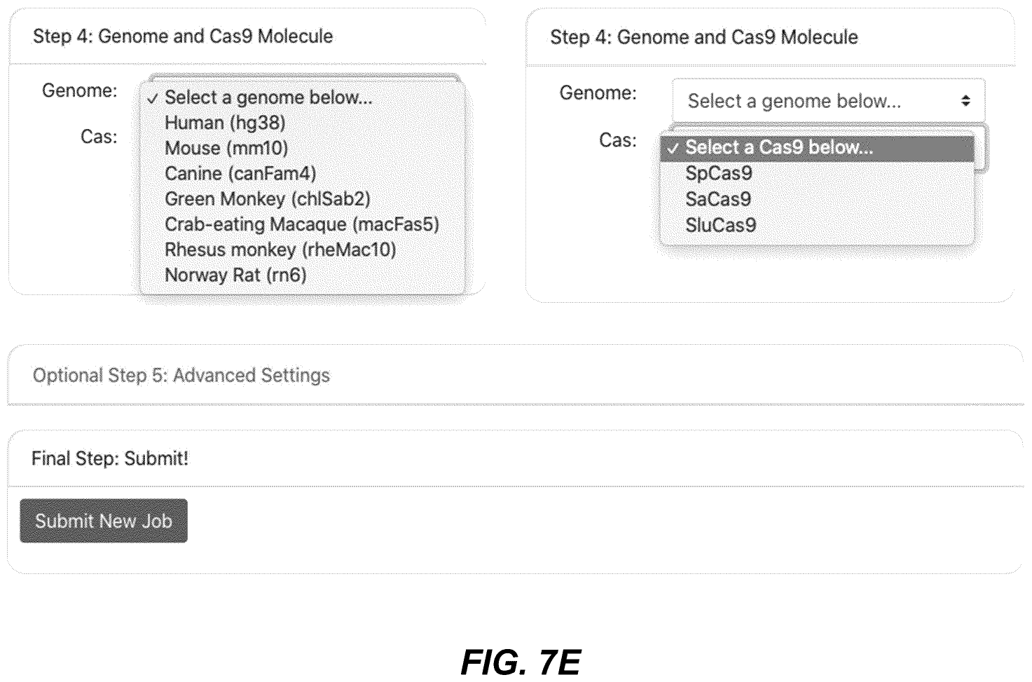

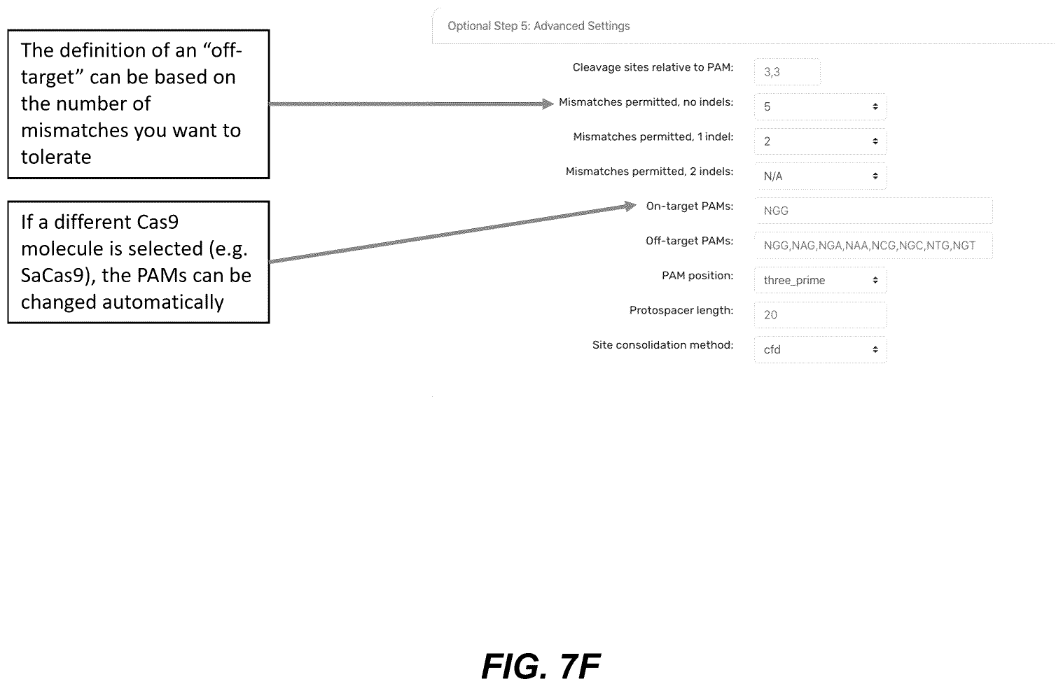

Described below is an exemplary use case for AVOLANCHE for finding best guides to disrupt an exon of a gene (FIG. 6). The sequence of a coding exon of a gene can be obtained from an online genome browser such as UCSC or Ensembl. The steps for using AVOLANCHE (FIG. 7A) can comprise the following: (1) Give the job a name (FIG. 7B); (2) Specify a use case (FIG. 7C), for example, in Case 1: Input is a sequence and results are potential guides or, in Case 2, a list of guide spacer sequences is provided by the user (without PAMs) and the results will just be the off-target profile of each guide; (3) Enter sequence (In some embodiments, sequences can be uploaded as, e.g., FASTA or CSV, FIG. 7D); (4) specify genome and Cas protein (FIG. 7E). In some embodiments, advanced parameters can be input (FIG. 7F).

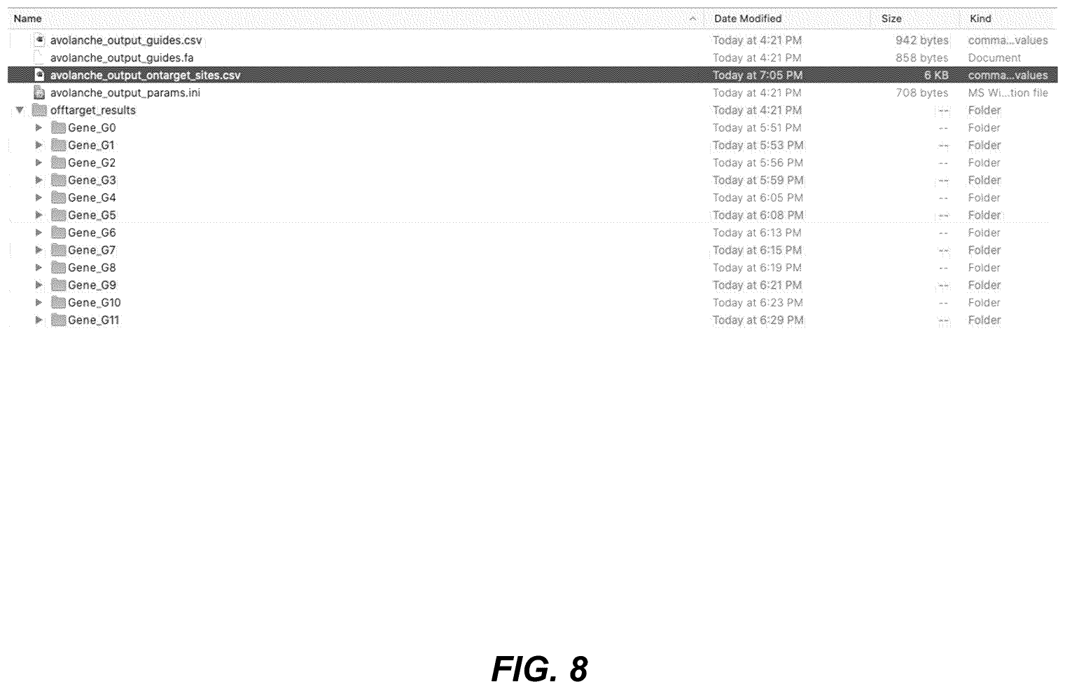

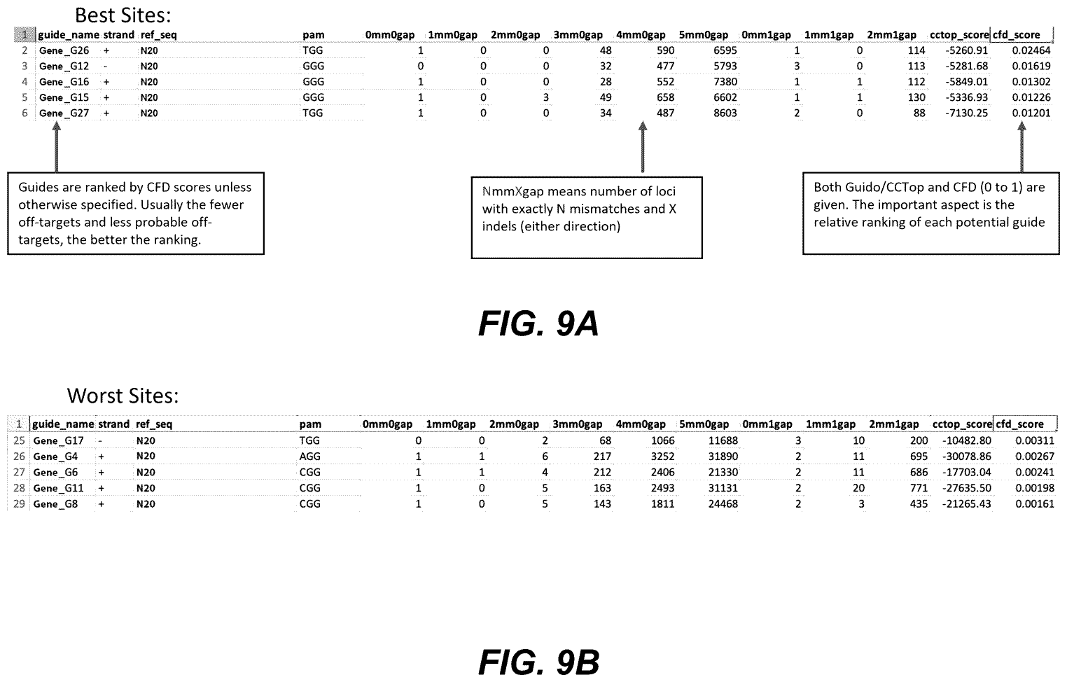

FIG. 8-FIG. 9B display exemplary output of the AVOLANCHE method. In some embodiments, the output can comprise the following: spreadsheet containing scores of all potential guides found (e.g., “avolanche_output_ontarget_sites.csv”); the guide sequences themselves (e.g., avolanche_output_guides (as, e.g., .fa, .csv)); debugging information (e.g., avolanche_output_params.ini); off target for each guide (G0, G1, G2, etc.) (e.g., offtarget_results).

In some embodiments, additional features of the algorithm can comprise: consolidation of overlapping off-target sites, on-target site SNP information, annotation of genes overlapped by sites, full support of Cas9 molecules with variable spacer lengths. In some embodiments, the web interface can be incorporated with other modular packages as part of a full, self-service pipeline. In some embodiments, the web interface can interface with a cloud application (e.g., Okta). In some embodiments, the web interface can comprise visualization.

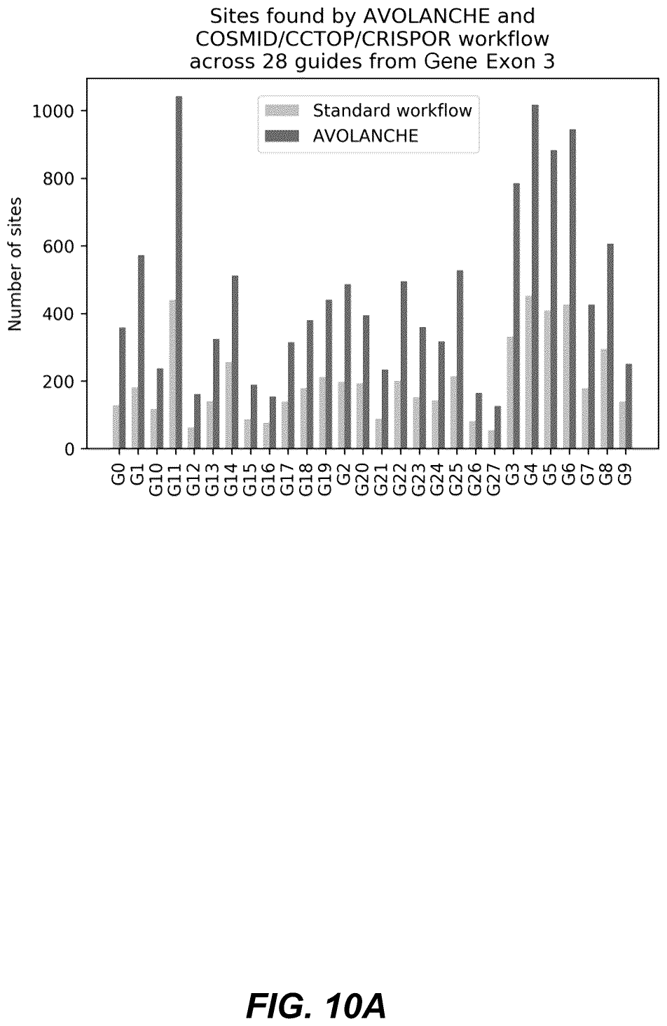

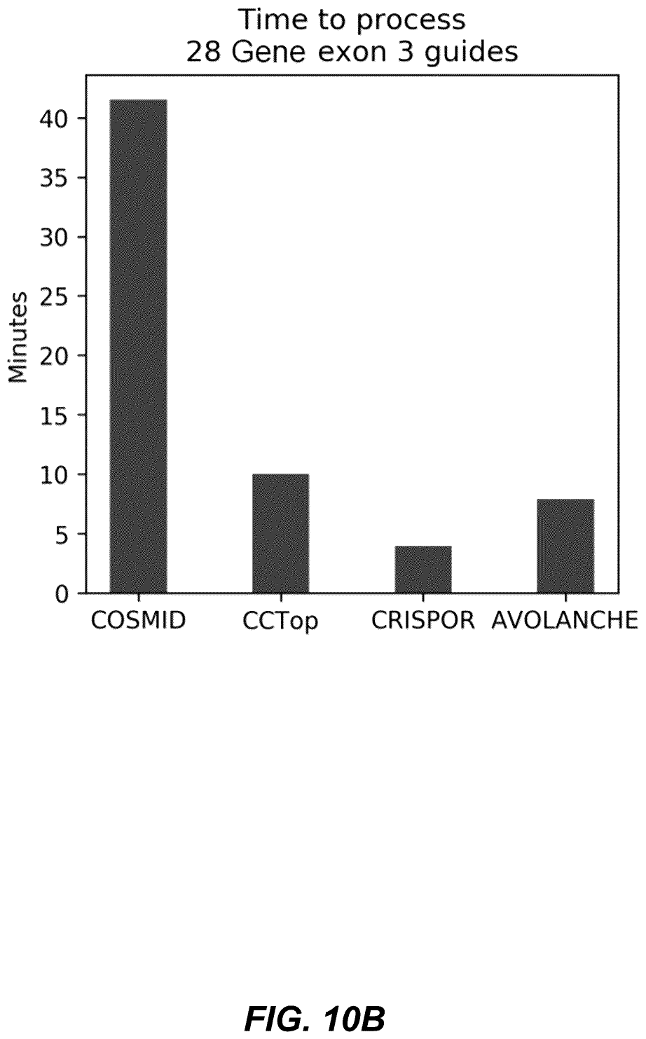

Described below are results from an exemplary use-case of the AVOLANCHE method provided herein. 28 SpCas9 guides from Gene exon 3 were designed. In an exemplary use-case, AVOLANCHE finds more sites (e.g., 3mm0gap, 2mm1gap; NGG, NAG, NGA, NAA, NCG, NGC, NTG, NGT off-target PAMs) than previous workflows and runs faster (FIG. 10A-FIG. 10B).

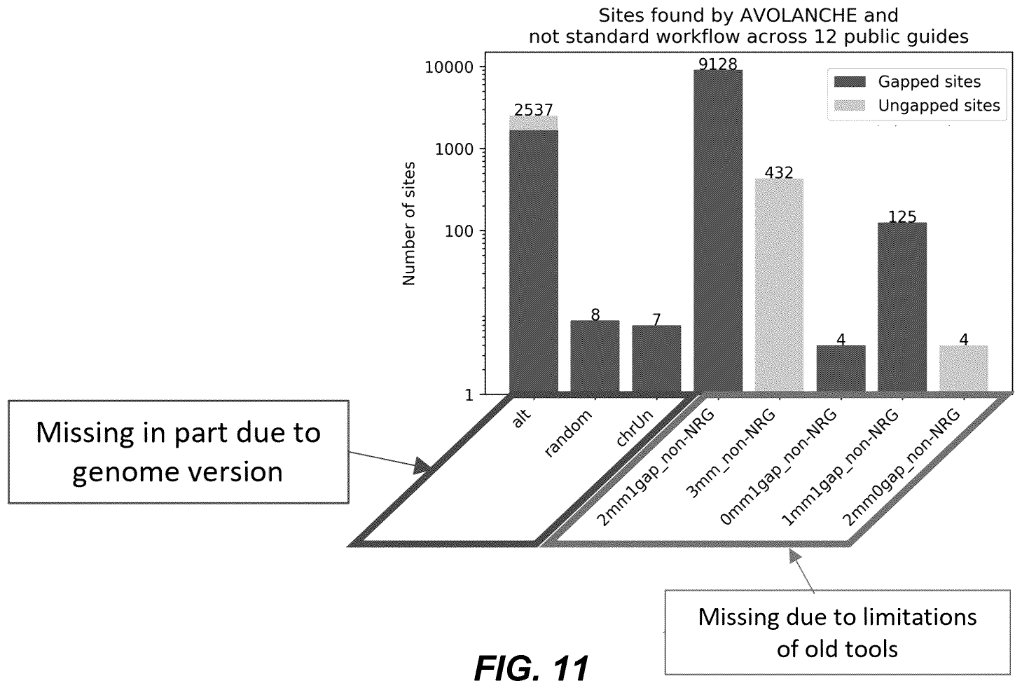

In a comparison between AVOLANCHE and previous tools, AVOLANCHE found additional off-target sites (FIG. 11). An off-target search using AVOLANCHE and old tools with 12 public guides, including several very dirty ones was run. AVOLANCHE and the old tools found 40,194 sites in common. AVOLANCHE found an additional 12,245 off-targets across the 12 guides.

In testing AVOLANCHE, it was validated that the homology sequences were being generated correctly. Determining the number of expected homology strings is a combinatorial problem, as shown in Equation 1 below:

$$S ( L , G , M ) = {\sum }_{g = 0}^{G} {\sum }_{d = 0}^{g} ( ( \begin{matrix}L \\ d\end{matrix} ) {\sum }_{m = 0}^{M} ( {3}^{m} ( \begin{matrix}L - d \\ m\end{matrix} ) ) {\sum }_{p = sgn ( g - d )}^{g - d} ( {4}^{( g - d )} ( \begin{matrix}L - 2 d + 1 \\ p\end{matrix} ) ) ) \qquad ( 1 )$$

where, S: number of expected sequences, L: length of the protospacer sequence, M: number of mismatches, G: number of gaps, d, p: subindexes. The number of expected sequences matched the number of sequences obtained for each homology space tested (Table 2).

TABLE 2
OBSERVED VS. EXPECTED SEQUENCES

| Homology space | Expected counts | Observed counts |
|---|---|---|
| 3mm0gap, 2mm1gap | 215,027 | 215,027 |
| 3mm0gap, 2mm1gap, | 545,438 | 545,438 |
| 1mm2gaps | | |
| 5mm0gap | 4,192,469 | 4,192,469 |
| 4mm0gap, 3mm1gap, | 13,174,643 | 13,174,643 |
| 2mm2gaps | | |

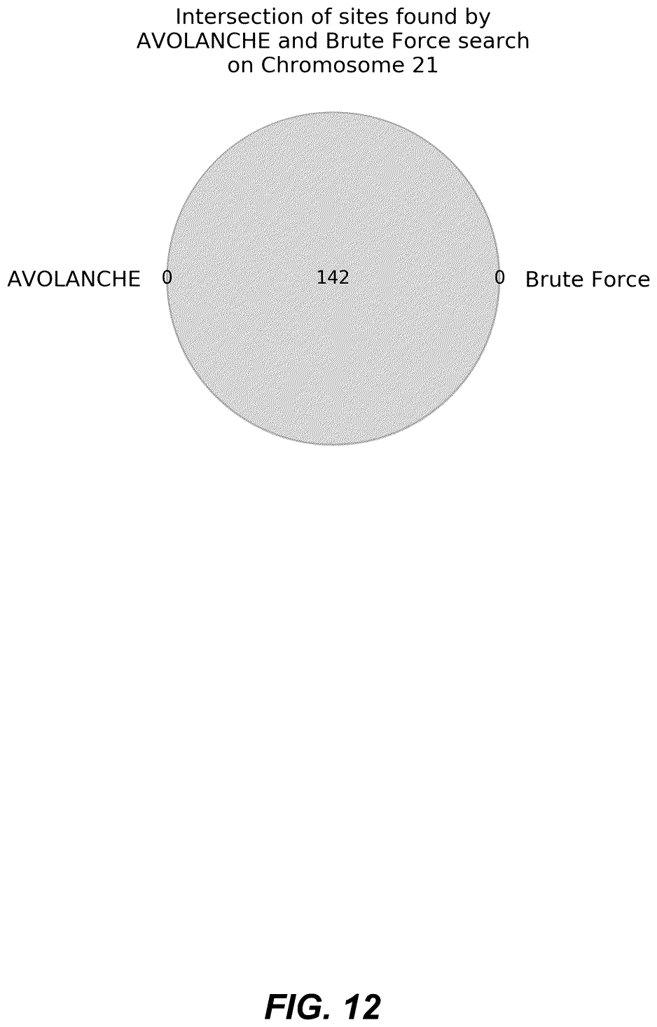

A brute-force algorithm was developed for scanning a chromosome base-by-base and finding off-targets. A brute-force search was performed to search for sites on Chr21 and compared to AVOLANCHE (Used 12 public guides; NRG PAM; 3mm0gap, 2mm1gap space). There is no evidence that AVOLANCHE misses sites that exist in the genome (FIG. 12). Table 3 below provides an exemplary summary of improvements to stages of guide development using AVOLANCHE.

TABLE 3
AVOLANCHE CAN IMPROVE ALL STAGES OF GUIDE DEVELOPMENT

| | | IND-enabling off- | BLA-enabling off- |
|---|---|---|---|
| Guide/Cas Design* | Off-target screening | target | target |
| Predict cleaner guides | Faster screen design. | Simplifies and de-risks | Allows for variant- |
| earlier. Avoid variant | Further de-risks guide | filings via | aware off-target to |
| off-targets | selection | comprehensive search | enable BLA filing |
| Predict cleaner guides | Faster screen design. | Simplifies and de-risks | |
| earlier. Enables exon | Further de-risks guide | filings via | |
| structure models. | selection | comprehensive search | |
| Avoid variant off- | | | |
| targets | | | |
| Predict cleaner guides | More comprehensive | Simplifies off-target | |
| earlier. Enables exon | search will de-risk | search for screening | |
| structure models. | WGS off-target | WGS indels | |
| Avoid variant off- | | | |
| targets | | | |
| Model organism | More comprehensive | Simplifies and de-risks | |
| genomes. PAMs for | search to de-risk in | filings via | |
| Cas orthologs. Avoid | vivo off-target | comprehensive search | |
| variant off-targets | | | |

*In some embodiments, column headers are stages of guide development displayed in temporal order from left to right.

### AVOLANCHE Testing Technical Summary

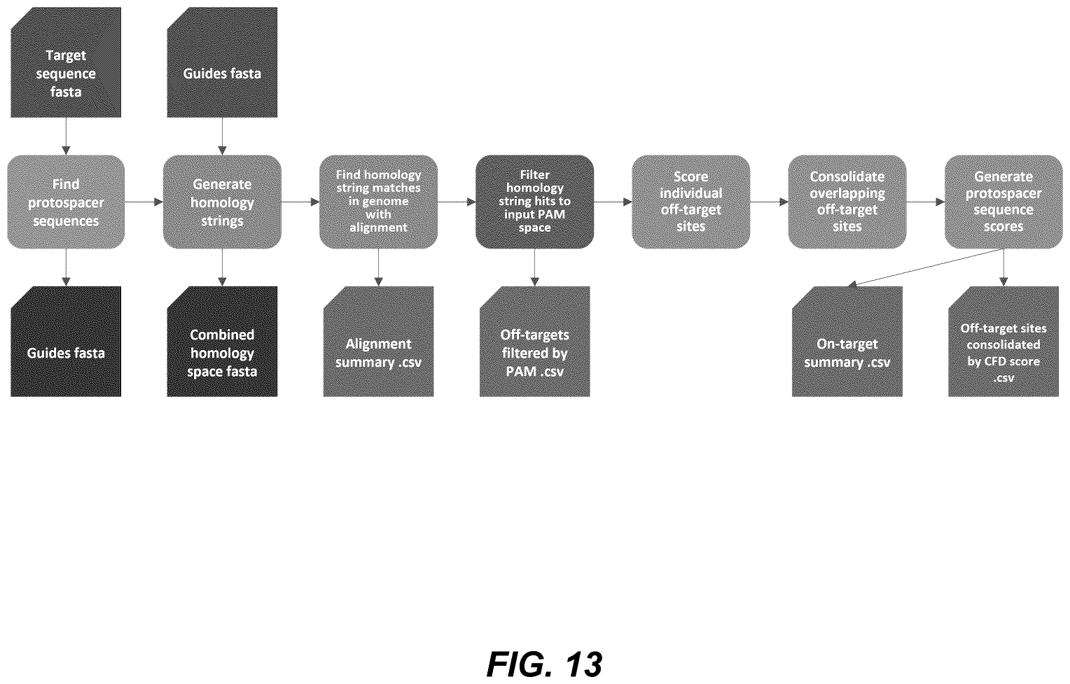

Described below is a technical summary of the methods described herein. FIG. 13 displays an exemplary workflow of the AVOLANCHE method. As described herein, AVOLANCHE generates the expected number of strings and the alignment can find every relevant site. Also provided are comparisons between the output of AVOLANCHE compared to a standard workflow.

### Homology Strings

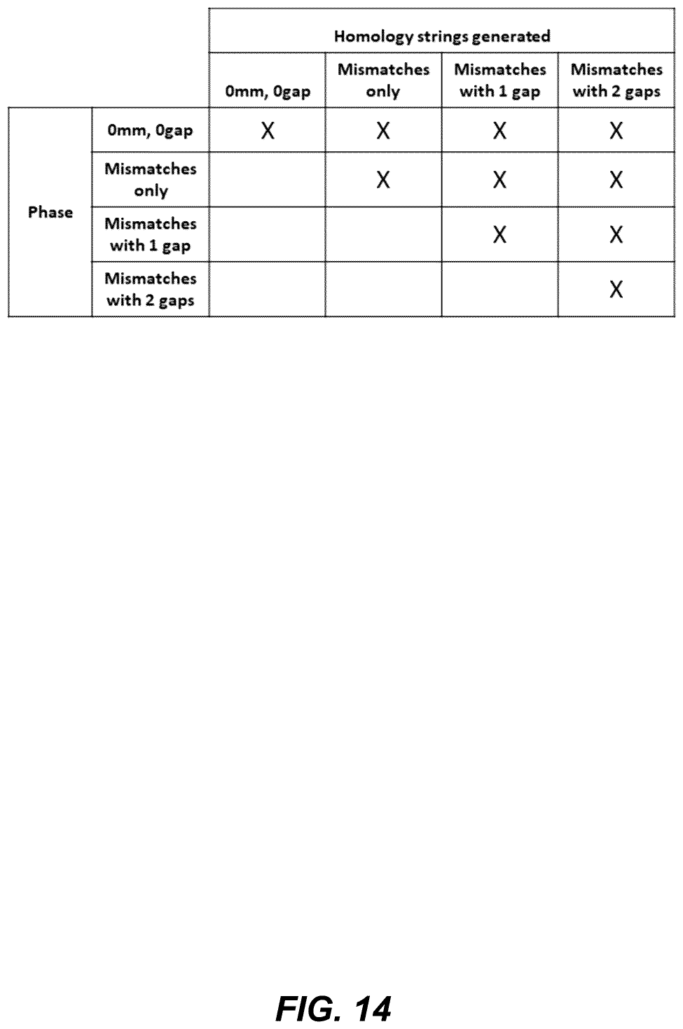

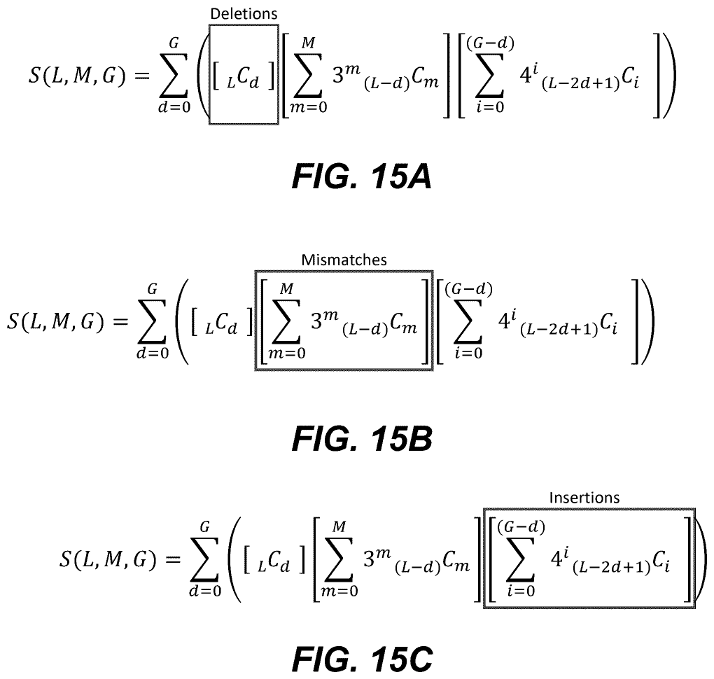

Homology string generation code can be run in, for example, four phases, a result of the input parameter structure (FIG. 14). Each phase generates all possible strings within its input homology space, leading to duplication of some strings. Calculating the number of expected homology strings for the 3mm0gap, 2mm1gap is a combinatorial problem (See, FIG. 15A-FIG. 15C). Shown below is a formula for calculating expected sequences for sequences with max 1 gap (Equation 2-4):

$$S ( L , G , M ) = {\sum }_{d = 0}^{G} ( [ {}_{L} {C}_{d} ] [ {\sum }_{m = 0}^{M} {3}_{( L - d )}^{m} {C}_{m} ] [ {\sum }_{i = 0}^{( G - d )} {4}_{( L - 2 d + 1 )}^{i} {C}_{i} ] ) \qquad ( 2 )$$

where S: number of expected sequences, L: length of the protospacer sequence, M: number of mismatches, G: number of gaps.

$$S ( L , G , M ) = {\sum }_{d = 0}^{G} ( [ {}_{L} {C}_{d} ] [ {\sum }_{m = 0}^{M} {3}_{( L - d )}^{m} {C}_{m} ] [ {\sum }_{i = 0}^{( G - d )} {4}_{( L - 2 d + 1 )}^{i} {C}_{i} ] ) + [ {\sum }_{m = 0}^{M} {3}_{( L - d )}^{m} {C}_{m} ] [ {\sum }_{i = 0}^{( G - d )} {4}_{( L - 2 d + 1 )}^{i} {C}_{i} ] \qquad ( 3 )$$

$$S ( L , G , M ) = {\sum }_{g = 0}^{G} ( {\sum }_{d = 0}^{g} [ {}_{L} {C}_{d} ] [ {\sum }_{m = 0}^{M} {3}_{( L - d )}^{m} {C}_{m} ] [ {\sum }_{i = 0}^{( g - d )} {4}_{( L + 2 G - d - 1 )}^{i} {C}_{i} ] ) \qquad ( 4 )$$

TABLE 4
EXPECTED STRING COUNTS
3mm0gap, 2mm1gap

| Phase | Inputs | S(L, M, G) |
|---|---|---|
| 0mm0gap | L = 20 | 1 |
| | M = 0 | |
| | G = 0 | |
| 3mm0gap | L = 20 | 32,551 |
| | M = 3 | |
| | G = 0 | |
| 2mm1gap | L = 20 | 182,475 |
| | M = 2 | |
| | G = 1 | |
| | Total | 215,027 |

An exemplary calculation is as follows: 1+3*(20choose1)+(3\hat{ }2)*(20choose2)+(3\hat{ }3)*(20choose3)+4*(21choose1)+(20choose1)+(4*(21choose1)*3*(20choose1))+((20choose1)*3*(19choose1))+(4*(21choose1)*(3\hat{ }2)*(20choose2))+((20choose1)*(3\hat{ }2)*(19choose2)).

TABLE 5A
EXPECTED STRING COUNTS
5mm0gap

| Phase | Inputs | S(L, M, G) |
|---|---|---|
| 0mm0gap | L = 20 | 1 |
| | M = 0 | |
| | G = 0 | |
| 5mm0gap | L = 20 | 4,192,468 |
| | M = 5 | |
| | G = 0 | |
| | Total | 4,192,469 |

TABLE 5B
EXPECTED STRING COUNTS
3mm0gap, 2mm1gap, 1mm2gaps

| Phase | Inputs | S(L, M, G) |
|---|---|---|
| 0mm0gap | L = 20 | 1 |
| | M = 0 | |
| | G = 0 | |
| 3mm0gap | L = 20 | 32,551 |
| | M = 3 | |
| | G = 0 | |
| 2mm1gap | L = 20 | 182,475 |
| | M = 2 | |
| | G = 1 | |
| 1mm2gap | L = 20 | 330,411 |
| | M = 1 | |
| | G = 2 | |
| | Total | 545,438 |

TABLE 5C
EXPECTED STRING COUNTS
4mm0gap, 3mm1gap, 2mm2gaps

| Phase | Inputs | S(L, M, G) |
|---|---|---|
| 0mm0gap | L = 20 | 1 |
| | M = 0 | |
| | G = 0 | |
| 4mm0gap | L = 20 | 424,996 |
| | M = 4 | |
| | G = 0 | |
| 3mm1gap | L = 20 | 3,322,035 |
| | M = 3 | |
| | G = 1 | |
| 2mm2gap | L = 20 | 8,960,315 |
| | M = 2 | |
| | G = 2 | |
| | Total | 13,267,679 |

Expected string counts for the 3mm0gap, 2mm1gap space matched those from the code for the 12 public guides. Described below is calculation of expected counts for a 3mm0gap, 2mm1gap space by phase: 0mm0gap, 1=₂₀C₀; 3mm0gap,

$$32551 = {\sum }_{r = 0}^{3} {3}^{r} \cdot {}_{20} {C}_{r} ; 2 mm 1 gap , 182475 = 4 \cdot {}_{21} {C}_{1} {\sum }_{r = 0}^{2} {3}^{r} \cdot {}_{20} {C}_{r} + {}_{20} {C}_{1} {\sum }_{r = 0}^{2} {3}^{r} \cdot {}_{19} {C}_{r} + {\sum }_{r = 0}^{2} {3}^{r} \cdot {}_{20} {C}_{r} ;$$

Total strings, 215027. Also, see Table 6 below.

TABLE 6
EXPECTED STRING COUNTS
3mm0gap, 2mm1gap

| Phase | Subspace counts | Subspace Total |
|---|---|---|
| 0mm0gap | 0mm0gap: 1 | 1 |
| 3mm0gap | 3mm0gap: 30,780 | 32,551 |
| | 2mm0gap: 1,710 | |
| | 1mm0gap: 60 | |
| | 0mm0gap: 1 | |
| 2mm1gap | 2mm1gap: 174,420 | 182,475 |
| | 1mm1gap: 6,180 | |
| | 0mm1gap: 104 | |
| | 2mm0gap: 1,710 | |
| | 1mm0gap: 60 | |
| | 0mm0gap: 1 | |
| | Total | 215,027 |

Expected string counts also match string counts obtained for other homology spaces (Table 7A-Table 7C).

TABLE 7A
EXPECTED STRING COUNTS
5mm0gap

| Phase | Subspace count | Subspace Total |
|---|---|---|
| 0mm0gap | 0mm0gap: 1 | 1 |
| 5mm0gap | 5mm0gap: 3,767,472 | 4,192,468 |
| | 4mm0gap: 392,445 | |
| | 3mm0gap: 30,780 | |
| | 2mm0gap: 1,710 | |
| | 1mm0gap: 60 | |
| | 0mm0gap: 1 | |
| | Total | 4,192,469 |

TABLE 7B
EXPECTED STRING COUNTS
3mm0gap, 2mm1gap, 1mm2gaps

| Phase | Subspace counts | Subspace Total |
|---|---|---|
| 0mm0gap | 0mm0gap: 1 | 1 |
| 3mm0gap | 3mm0gap: 30,780 | 32,551 |
| | 2mm0gap: 1,710 | |
| | 1mm0gap: 60 | |
| | 0mm0gap: 1 | |
| 2mm1gap | 2mm1gap: 174,420 | 182,475 |
| | 1mm1gap: 6,180 | |
| | 0mm1gap: 104 | |
| | 2mm0gap: 1,710 | |
| | 1mm0gap: 60 | |
| | 0mm0gap: 1 | |
| 1mm2gap | 1mm2gap sites: 318,660 | 330,411 |
| | 1mm1gap sites: 6,180 | |
| | 0mm2gap sites: 5,406 | |
| | 0mm1gap sites: 104 | |
| | 1mm0gap sites: 60 | |
| | 0mm0gap sites: 1 | |
| | Total | 545,438 |

TABLE 7C
EXPECTED STRING COUNTS
4mm0gap, 3mm1gap, 2mm2gaps

| Phase | Subspace counts | Subspace Total |
|---|---|---|
| 0mm0gap | 0mm0gap: 1 | 1 |
| 4mm0gap | 4mm sites: 392,445 | 424,996 |
| | 3mm0gap: 30,780 | |
| | 2mm0gap: 1,710 | |
| | 1mm0gap: 60 | |
| | 0mm0gap: 1 | |
| 3mm1gap | 3mm1gap sites: 3,108,780 | 3,322,035 |
| | 2mm1gap sites: 174,420 | |
| | 1mm1gap sites: 6,180 | |
| | 1gap sites: 104 | |
| | 3mm sites: 30,780 | |
| | 2mm sites: 1,710 | |
| | 1mm sites: 60 | |
| | 0mm0gap sites: 1 | |
| 2mm2gap | 2mm2gap sites: 8,921,070 | 9,427,611 |
| | 1mm2gap sites: 318,660 | |
| | 2mm1gap sites: 174,420 | |
| | 1mm1gap sites: 6,180 | |
| | 2gap sites: 5,406 | |
| | 1 gap sites: 104 | |
| | 2mm sites: 1,710 | |
| | 1mm sites: 60 | |
| | 0mm0gap sites: 1 | |
| | Total | 13,174,643 |

### AVOLANCHE Finds all Relevant Sites

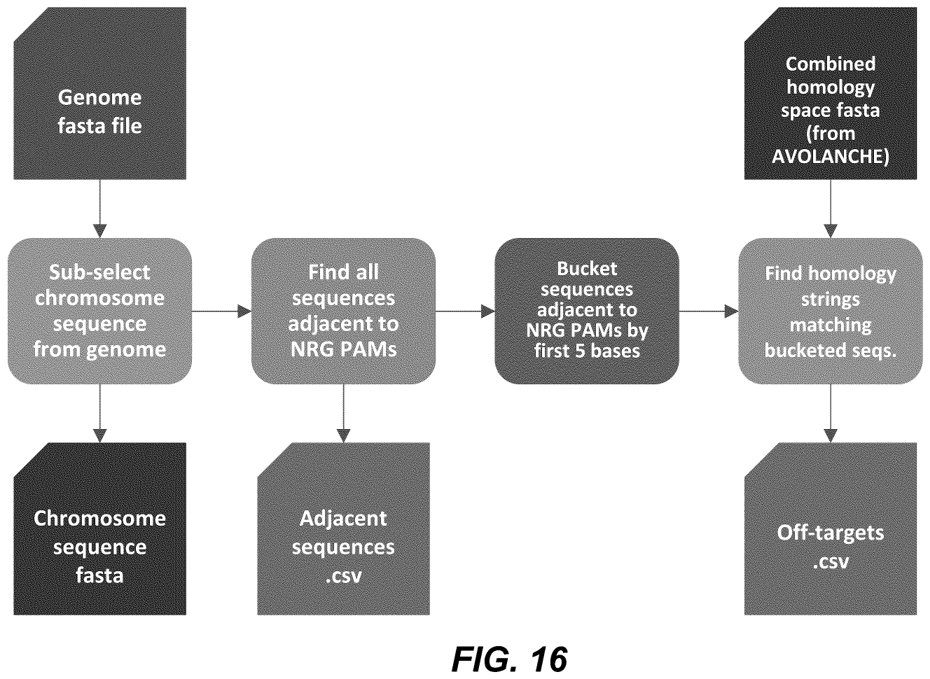

12 public guides were run using AVOLANCHE and a brute-force search (NRG PAM; 3mm0gap, 2mm1gap). Due to the excessive time and memory it takes to run the brute-force search, off-target results were computed for chromosome 21. The brute-force search was built to validate that the alignment output wasn't missing off-target sequences (FIG. 16). As shown in FIG. 12, AVOLANCHE and the brute force search found the exact same sites on chromosome 21.

### Comparison to Previous Methods

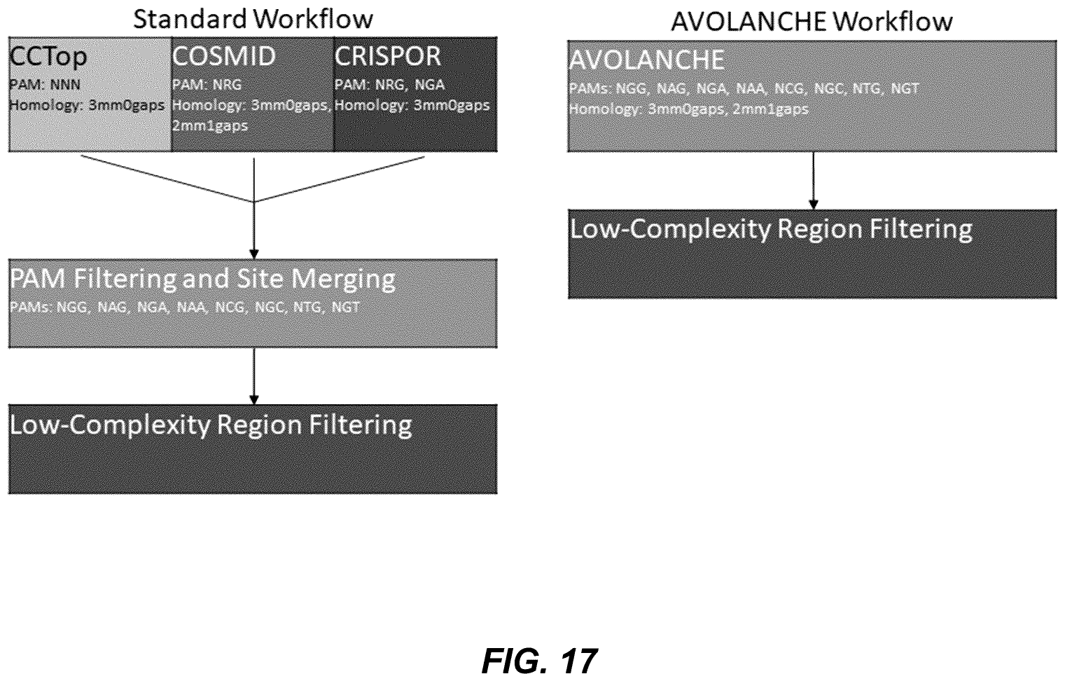

To determine if AVOLANCHE can find more sites than previous methods, 12 public guides were run with the standard off-target workflow and AVOLANCHE using comparable inputs (FIG. 17). The tools in the standard workflow have several known limitations (Table 8).

TABLE 8
LIMITATIONS OF STANDARD WORKFLOWS

| CCTop | CRISPOR | COSMID |
|---|---|---|
| Cannot identify sites with | Cannot identify sites with | hg38 lacks alternative |
| gaps; | gaps; | chromosomes; |
| The two PAM-adjacent | Only tracks NRG, NGA | Includes PAM mismatches in |
| bases cannot contain | PAMs; | the total mismatches |
| mismatches | Automated score-based | |
| | filtering | |

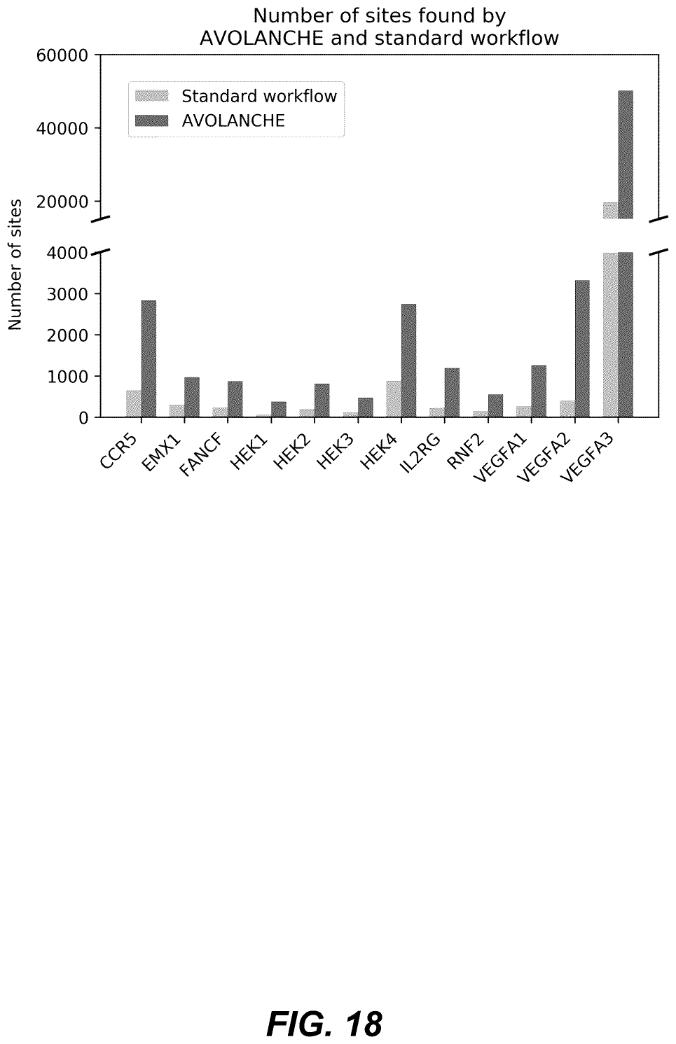

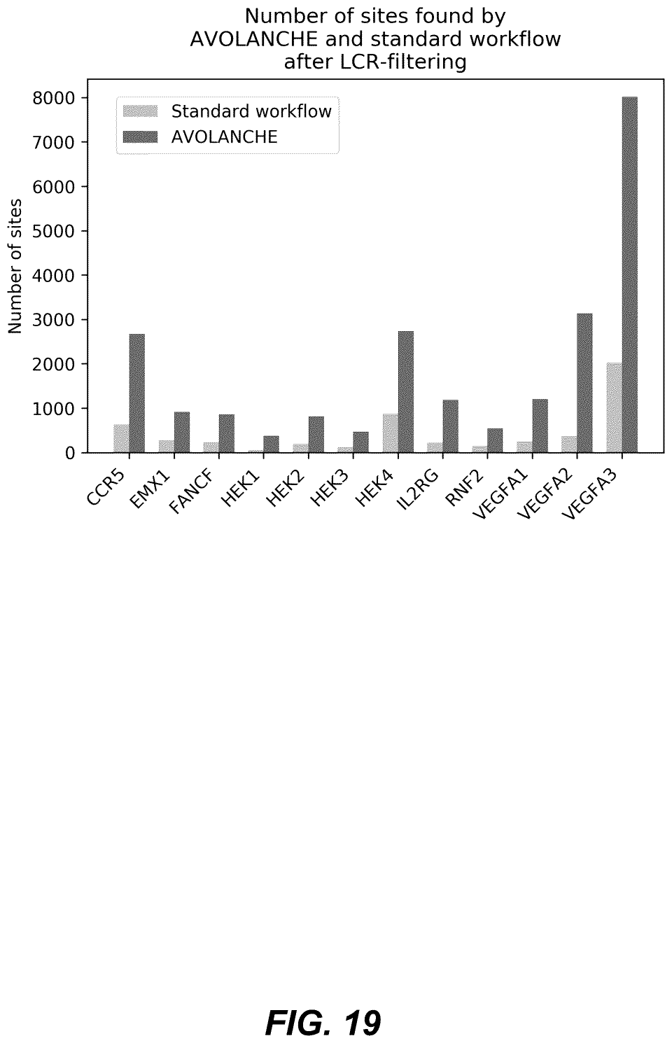

As shown in FIG. 18, AVOLANCHE found more sites than the standard workflow for every guide. The standard workflow found 23,318 total sites. In contrast, AVOLANCHE found 65,475 total sites (49,062 unique genomic coordinates). After removing low-complexity regions (LCRs), AVOLANCHE still found more sites than the standard workflow for every guide. As shown in FIG. 19, standard workflow found 5,462 sites not overlapping an LCR, while AVOLANCHE found 22,923 (15,688) sites not overlapping an LCR.

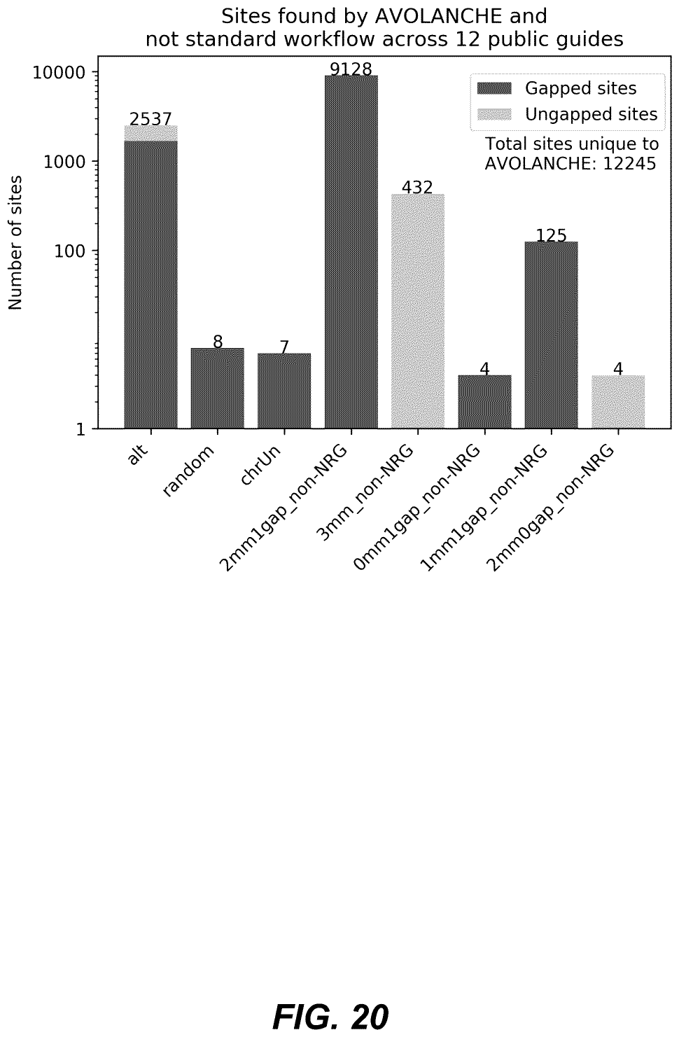

AVOLANCHE found 12,245 (8,868) sites that do not overlap any site found by the standard workflow (FIG. 20). The genome used by AVOLANCHE accounts for some of the sites not found by COSMID in the standard workflow. COSMID's copy of hg38 lacks alternative chromosomes. This accounts for 2,552 (1,834) of the 12,245 sites not found (See, e.g., first 3 bars of graph shown in FIG. 20).

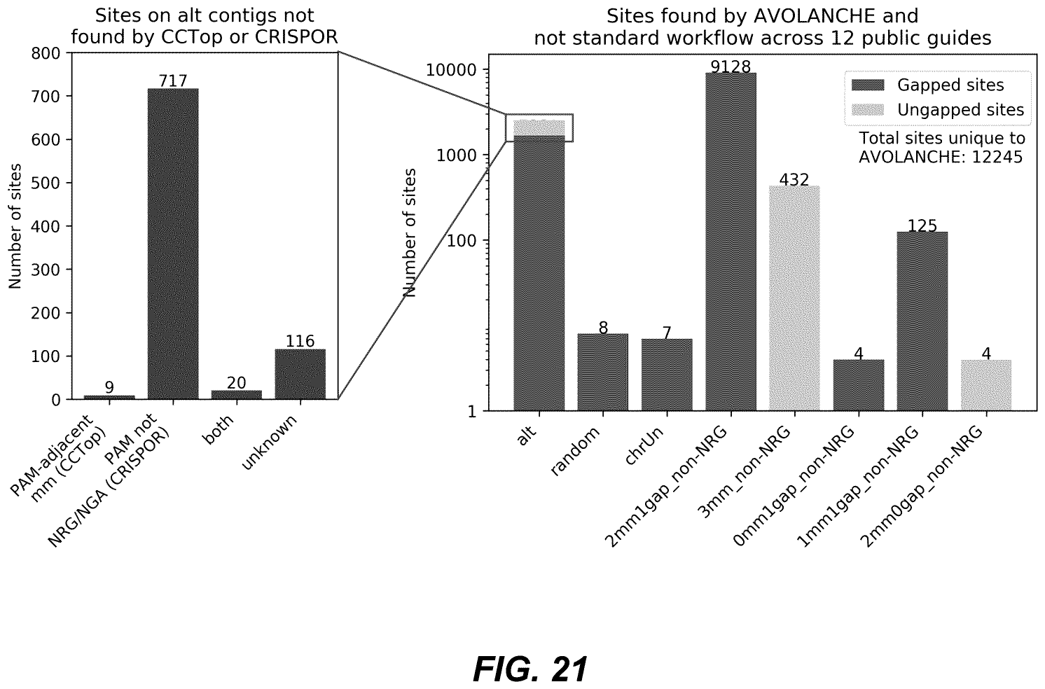

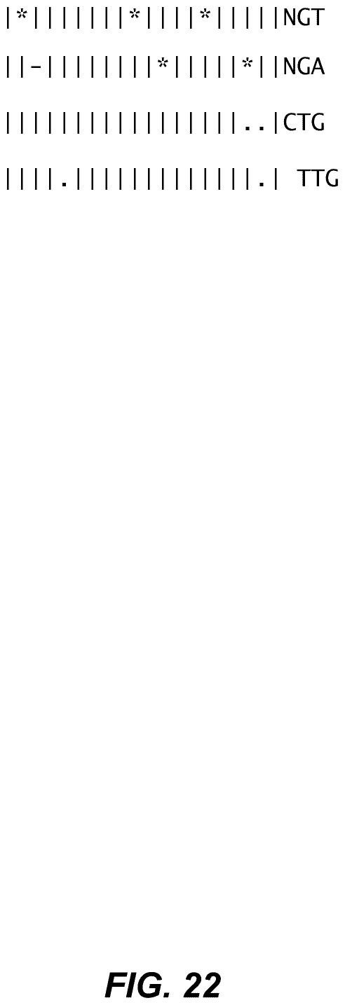

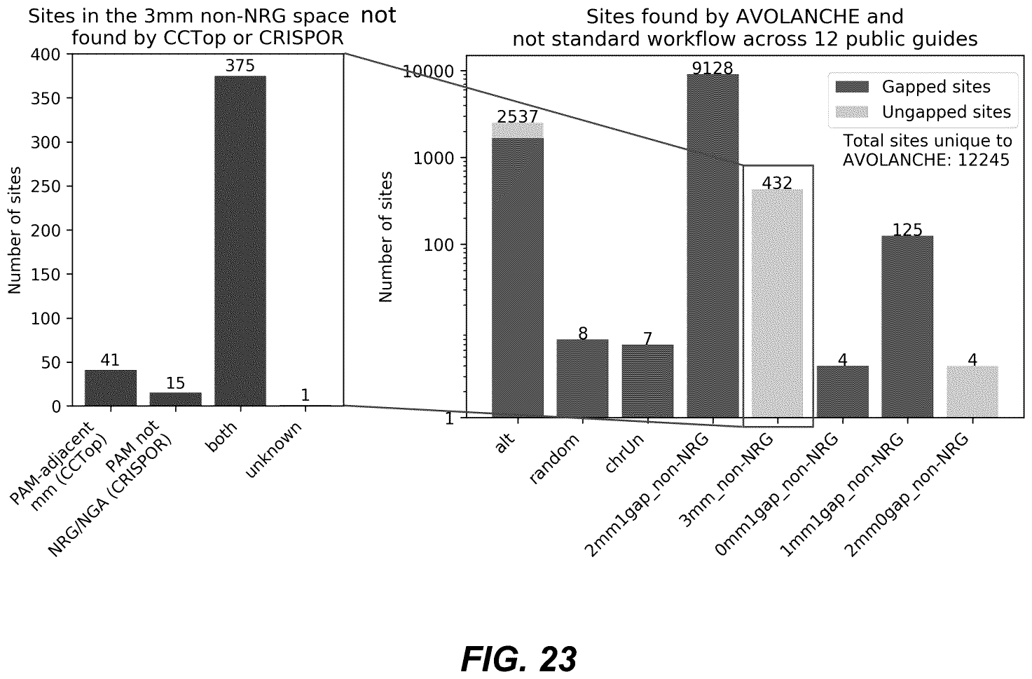

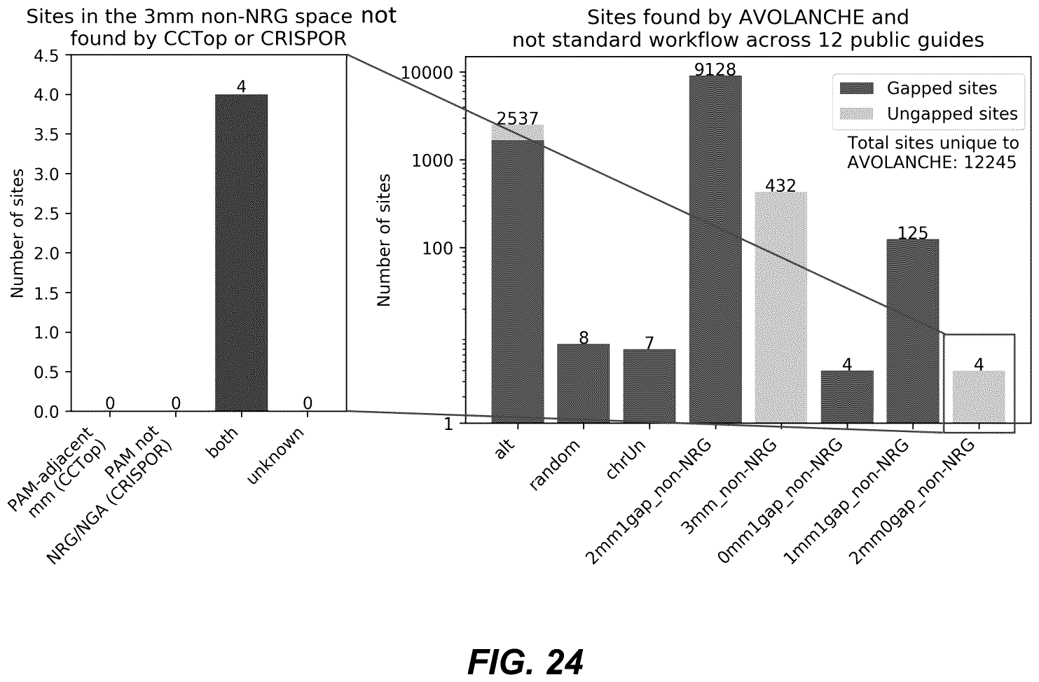

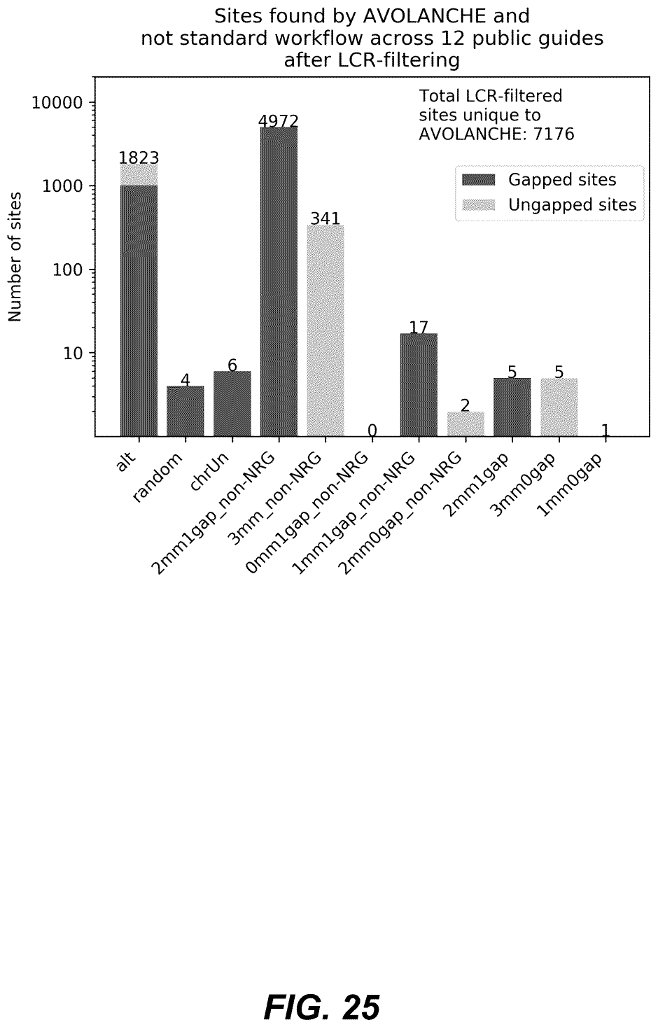

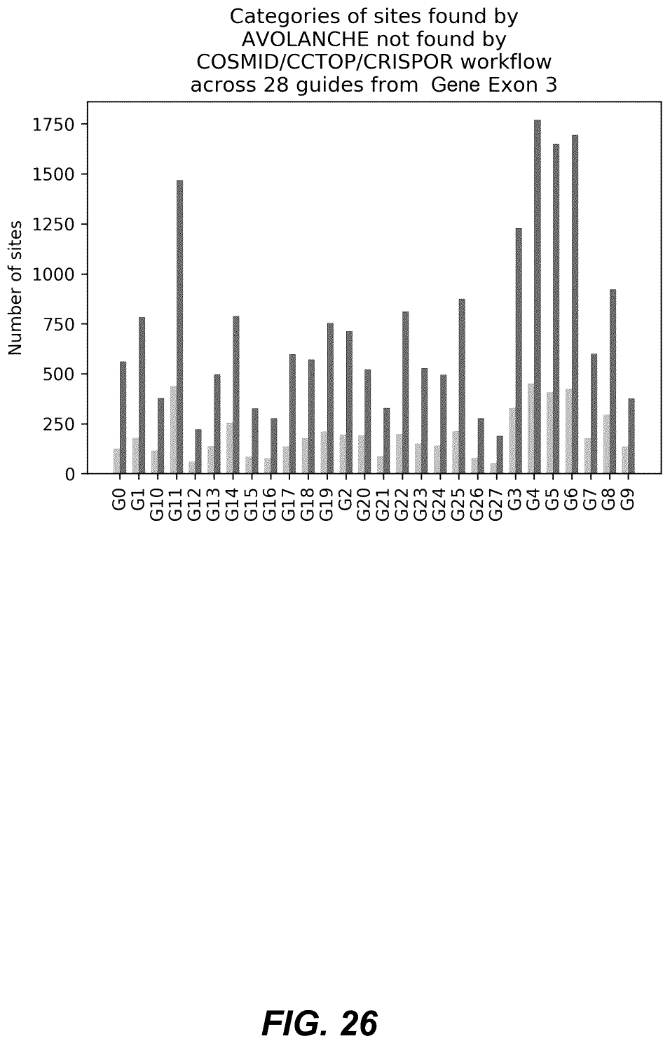

CCTop and CRISPOR missed 862 (826) ungapped sites on haplotype chromosomes (FIG. 21). COSMID missed 2mm1gap sites with non-NRG PAMs. The standard workflow missed 9,128 (6,600) sites with 2mm1gap and non-NRG PAMs (See, e.g., “2mm1gap_non-NRG” bar of graph shown in FIG. 20). COSMID misses sites at edge of homology space with non-NRG PAMs (FIG. 22). COSMID also missed 3 mm sites with non-NRG PAMs. The standard workflow missed 432 (432) sites with 3mm0gap and non-NRG PAMs (See, e.g., “3 mm_non-NRG” bar in graph shown in FIG. 20). CCTop and CRISPOR also missed these 3 mm sites (FIG. 23). PAM filtering at multiple steps removed sites that could have been found by COSMID. 133 (100) sites were missed due to COSMID's internal PAM filtering or were removed during the filter and merge step (See, e.g., last three bars of graph shown in FIG. 20). When “R” is specified in PAM, that position is locked into “A” or “G”. COSMID did find sites with “NYN” PAMs, but only 3 cases out of 23,318 total results, all with deletions at the “R”. AVOLANCHE found 5,469 sites with “NYN” PAMs. Acceptable workflow PAMs can comprise: NGG, NAG, NGA, NAA, NCG, NGC, NTG, NGT. 2 sites were found by COSMID but reported with GAT and GAC PAMs, so the filter and merge step removed them. CCTop and CRISPOR missed 4 (2) 2mm0gap sites due to known limitations (FIG. 24). After LCR-filtering, AVOLANCHE found 7,176 (7,176) sites that do not overlap any site found by the standard workflow (FIG. 25). Discrepancies in the coordinates reported by the two workflows caused 11 sites to be differentially filtered (See, e.g., last 3 bars of graph shown in FIG. 25).

For testing AVOLANCHE with LCR-filtering, 28 guides targeting Gene exon 3 were designed and used for testing (NGG, NAG, NGA, NAA, NCG, NGC, NTG, NGT PAMs; 3mm0gap, 2mm1gap). AVOLANCHE found more sites than the standard workflow for all 28 guides before site consolidation. The standard workflow found 5,591 sites across the 28 guides and 5,532 after LCR-filtering. AVOLANCHE found 20,227 (13,971) sites across the 28 guides and 13,258 after LCR-filtering. AVOLANCHE found more sites than the standard workflow for all 28 guides after site consolidation (FIG. 10A, Table 9). AVOLANCHE is faster than the standard workflow for the Gene use case (FIG. 10B). Taking top 10 guides (by lowest sites) for a hybrid capture guide screen would get 994 sites with standard workflow and 2150 with AVOLANCHE.

TABLE 9
COMPARISON AFTER LCR FILTERING

| | Standard workflow | AVOLANCHE |
|---|---|---|
| Total sites | 5,591 | 12,703 |
| After LCR-filtering | 5,532 | 12,589 |
| Median sites per guide | 179.5 | 387.5 |

As discussed above, COSMID is the bottleneck of the standard workflow. As described herein, AVOLANCHE outperforms the current gold-standard, COSMID, and the standard workflow in general. AVOLANCHE is faster, more easily maintained and updated, comprises a modular architecture, can be written in python (e.g., and not Perl); can use a modern aligner (e.g., bwa) with wider community acceptance, and can be more easily configured for larger homology spaces.

In some embodiments, two options are provided: (1) Start a new EC2 instance using, e.g., the avolanche_0.0.0_200515 AMI; (2) On an instance with your own AMI that has Anaconda installed, clone the avolanche repository and run conda env create-f avolanche/avolanche_env.yml. The user can start the conda environment with, e.g., conda activate avolanche_env. The user can then set up input files and run jobs.

### AVOLANCHE Site Consolidation

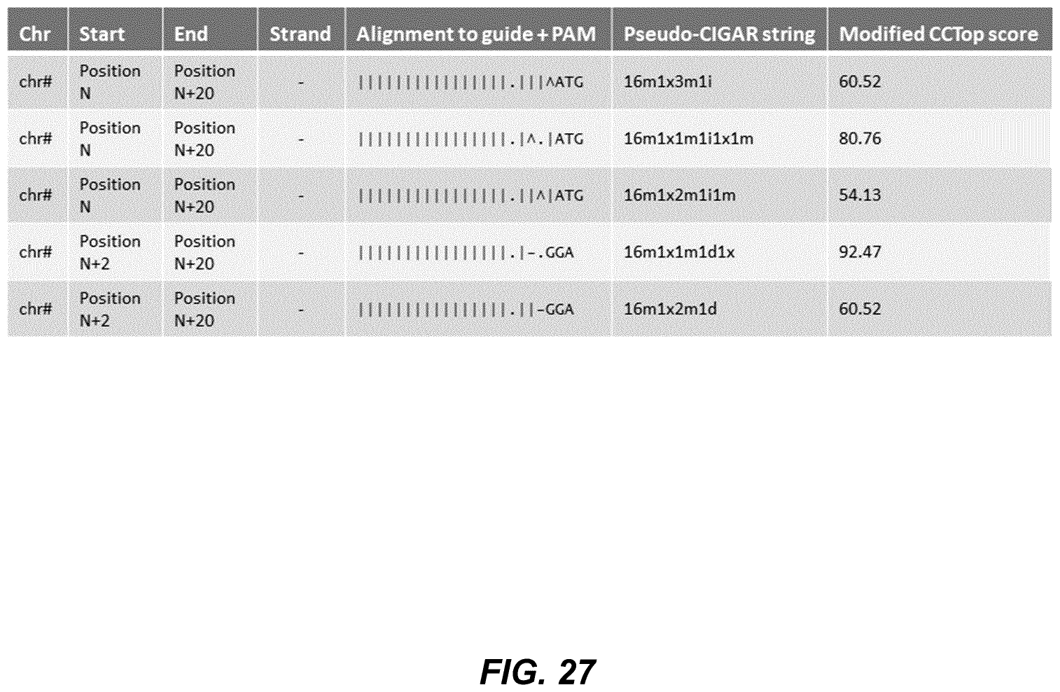

In some embodiments, AVOLANCHE performs a step consolidating overlapping off-target sites prior to reporting the finalized outputs. Sites get consolidated to remove those with multiple possible alignments. In some embodiments, AVOLANCHE finds sites with many possible alignments. In an exemplary case shown in FIG. 27, 5 alignments are found at chr #: position N-position (N+20).

Several different options exist for implementing site consolidation and are listed below (in order from less conservative to more conservative): Consolidate sites with a certain threshold of overlap; Consolidate sites with the same start OR end coordinate; Consolidate sites with the same PAM location and the same start OR end coordinate; Consolidate on PAM coordinates; Consolidate sites with same cut and start coordinates; Consolidate sites with the same start and end coordinates; No site consolidation—report all sites.

In some embodiments, sites with the same PAM coordinates can be consolidated. In some embodiments, this can be easy to implement and simple to explain. Two sites are reported in the example based on exemplary rules (See, FIG. 27, rows 3 and 5).

In some embodiments, the reference version of the human genome (e.g., hg38) contains contigs that can confound off-target analysis: _alt: alternative contigs representing common complex variation; chrUn_: contigs of unknown chromosomal origin; _random: contigs of known chromosomal origin, with unknown position; Pseudoautosomal regions: regions on the X and Y chromosomes with the same sequences; EBV and decoy contigs*: contigs to siphon off reads from EBV and some repetitive sequences (*Not found in current recommended hg38 version used for one implementation of AVOLANCHE).

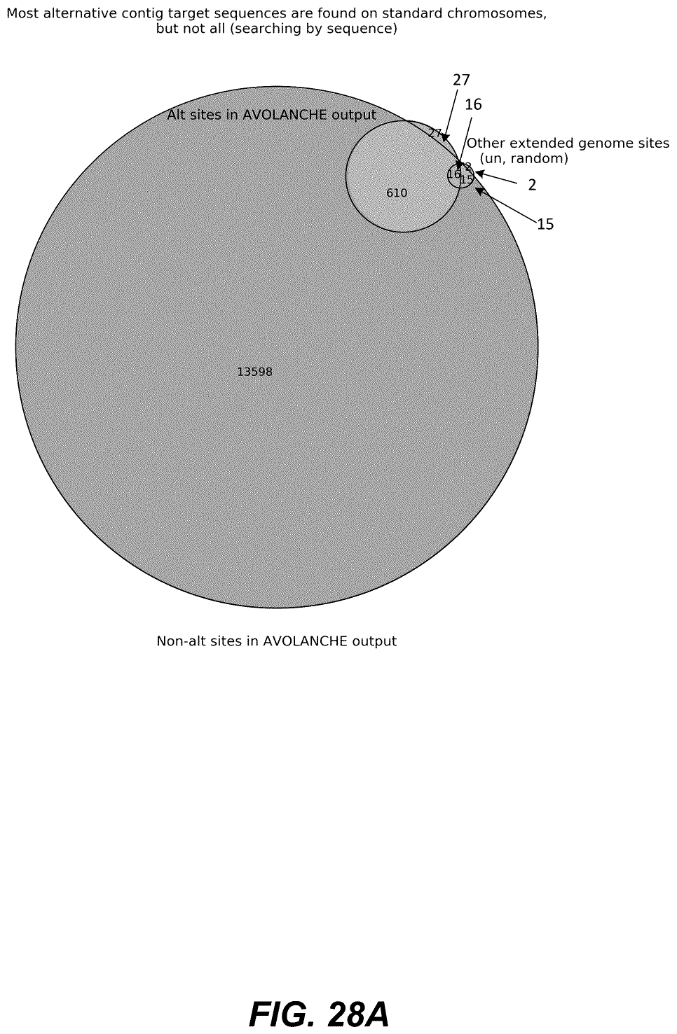

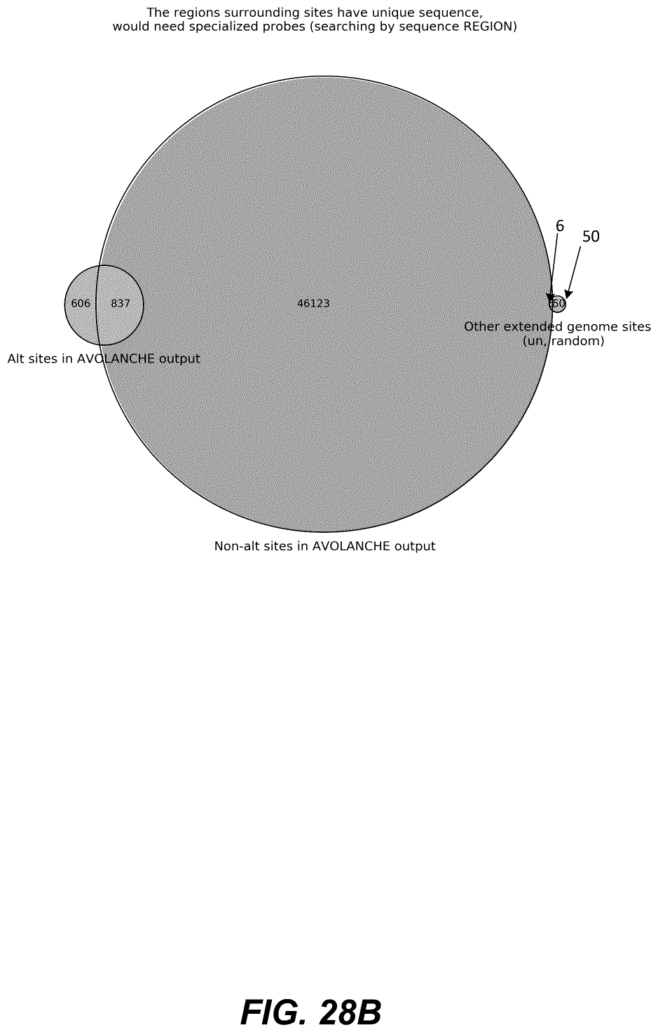

As shown in FIG. 28A-FIG. 28B, alternative chromosome sites are not 100% redundant in two different AVOLANCHE-generated data sets. In some embodiments, the specific target sites are mostly found on other chromosomes (FIG. 28A). In some embodiment, the sequences around them (+/−100 bp) are more unique (FIG. 28B). This could, in some embodiments, require probes.

Consolidation options for, e.g., probe design and regulatory reporting are shown in Table 10 below.

TABLE 10
CONSOLIDATION SUMMARY

| | | | | |
|---|---|---|---|---|
| | | Hybrid | | |
| Consolidation | Consolidation | capture probe | Hybrid capture | Regulatory |
| option | heuristic | design | indel analysis | reporting |

Site consolidation

| | | | |
|---|---|---|---|
| 1 | Consolidate by PAM | select either | select either this |
| | location, then CFD | this or #2 | or #2 |
| | score | | |
| 2 | Consolidate by PAM | Y^{†} | Y |
| | location, then | | |
| | minimum homology*, | | |
| | then CFD score | | |
| 3 | Consolidate by PAM | Y, in vivo | Y, in vivo |
| | location, then by | | |
| | CCTop (or SCAM- | | |
| | seq)*** | | |

Region consolidation

| | | | |
|---|---|---|---|
| 4 | Consolidate by | Y | Y |
| | regional proximity** | | |

*Minimum edit distance, followed by fewer gaps [consider using only for homology space comparisons?]
**5 bp buffer to create a site ‘group’, then select representative based on #2 or #1
***only for non-Sp Cas9 proteins
^{†}Y, option selection

Additional site consolidation options and output files can include: (1) Consolidate sites with the same PAM coordinates, reporting the alignment that's most likely to cut; (2) Consolidate with a hierarchical rule-based system of homology at same PAM coordinates, and then by alignment that's most likely to cut (e.g. 1 mm sites take priority over gap sites, etc.); (3) Consolidate proximal sites with a certain threshold of overlap.

### AVOLANCHE and LCR Filter

To allow the LCR Filter step to automatically be configured to run (if requested) after an AVOLANCHE job finishes, on the website, there can be, in some embodiments, two approaches: (1) The one web app approach—The AVOLANCHE HELIX app will let users apply a further stop (e.g., LCR Filter); (2) The integrated multiple web apps approach—A separate LCR Filter HELIX app integrates with other HELIX apps such as AVOLANCHE and allows it to use inputs directly from there. In some embodiments, the approach will impact other programs/applets beyond LCR Filter.

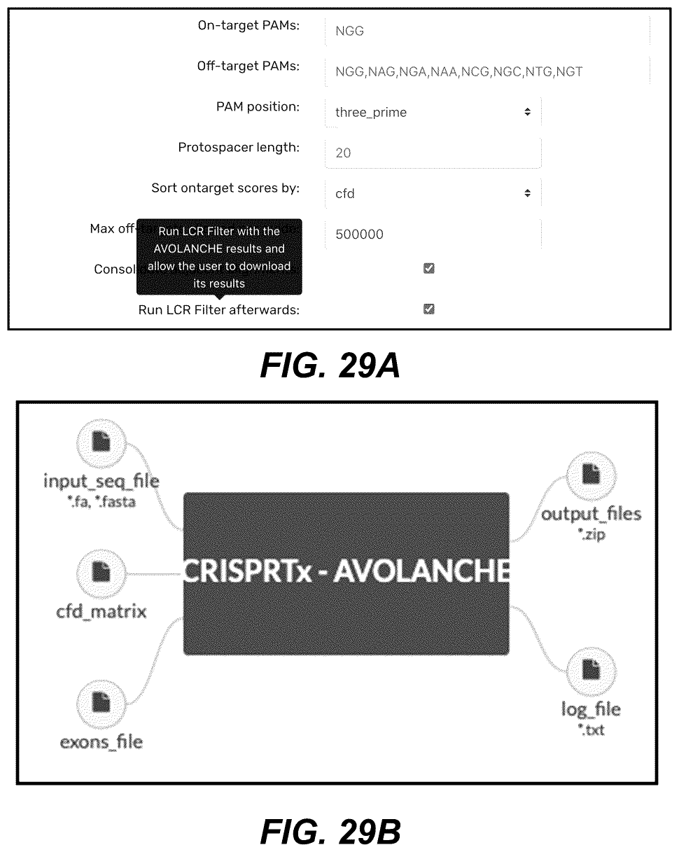

For a single web-app approach, in some embodiments, the AVOLANCHE web-app starts a DNANexus applet job when a new job is submitted. If the LCR Filter checkbox is checked, instead of an applet being launched, a separate webflow consisting of multiple applets (AVOLANCHE and LCR Filter) can be launched (FIG. 29A-FIG. 29B). This may advantageously provide an easier workflow for end user and be faster to iterate.

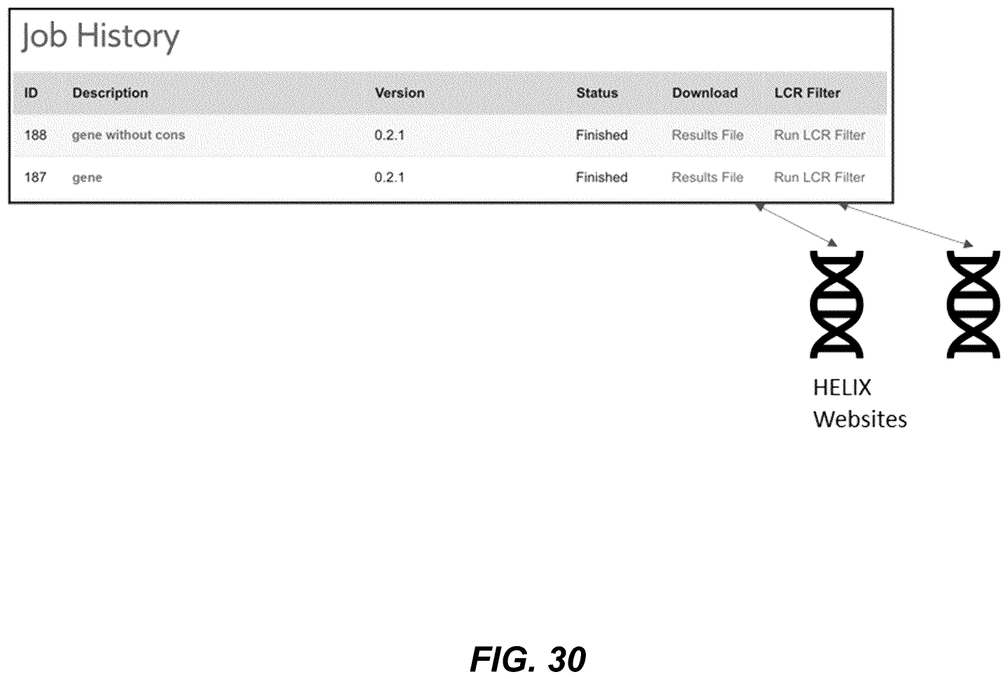

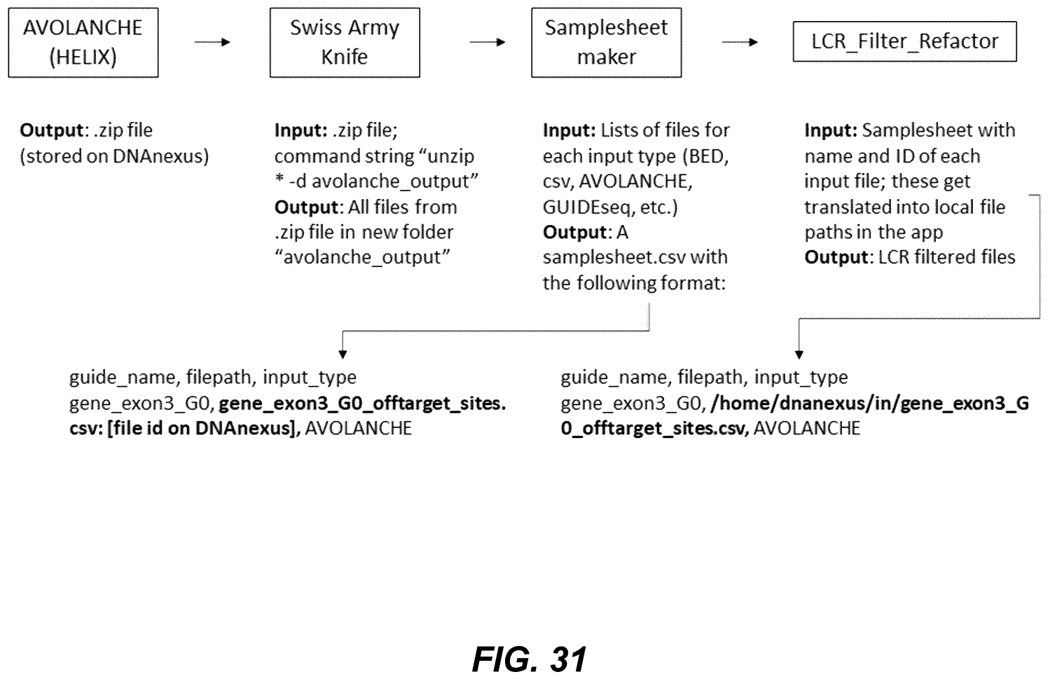

Under a multi web-app approach (FIG. 30), a separate LCR Filter HELIX app can be granted access to the completed AVOLANCHE web app jobs (and vice versa) and it can use the AVOLANCHE outputs as inputs. This would advantageously not need to define workflows ahead of time and/or compose new pipelines on the spot.

### Determining Protospacer Profiles

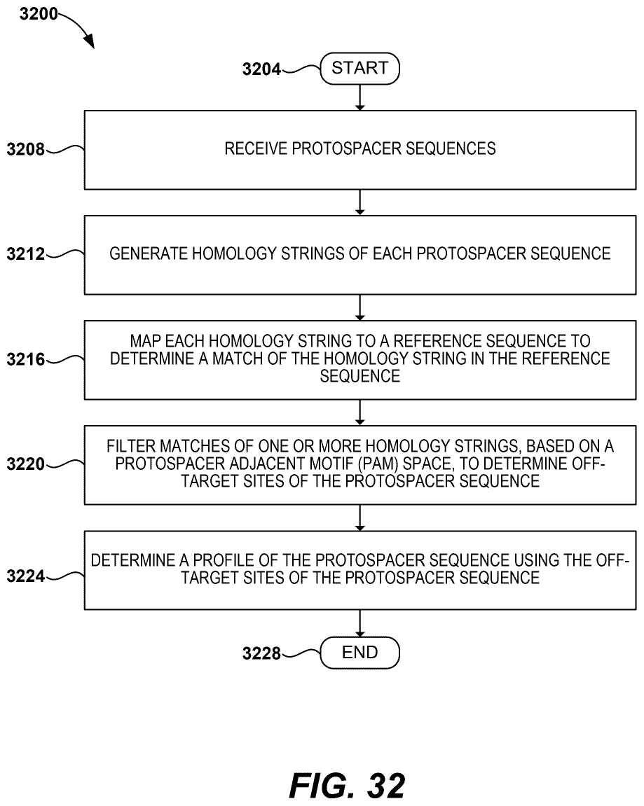

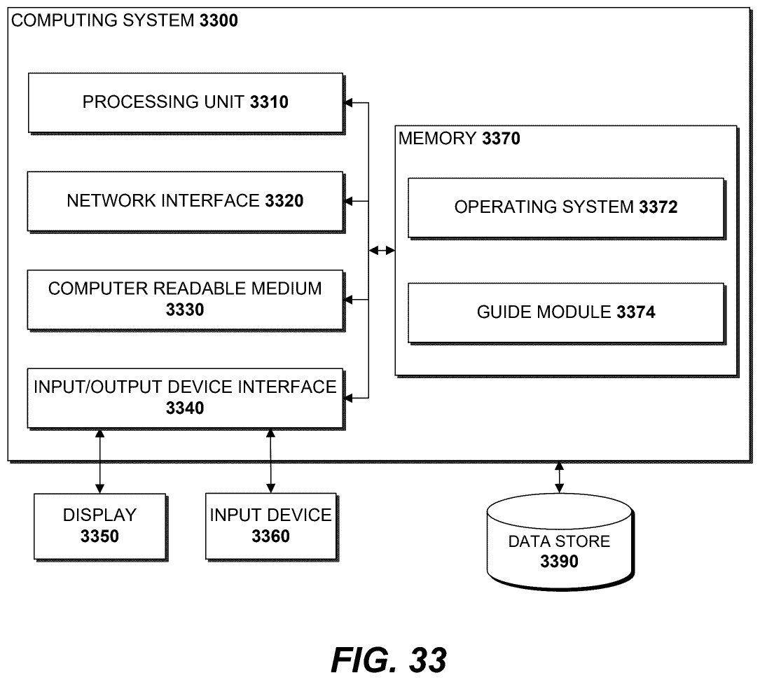

FIG. 32 is a flow diagram showing an exemplary method 3200 of determining protospacer sequence profiles (or selecting one or more protospacer sequences, off-target prediction, or guide design). The method 3200 (or a portion thereof) may be embodied in a set of executable program instructions stored on a computer-readable medium, such as one or more disk drives, of a computing system. For example, the computing system 3300 shown in FIG. 33 and described in greater detail below can execute a set of executable program instructions to implement the method 3200. When the method 3200 (or a portion thereof) is initiated, the executable program instructions can be loaded into memory, such as RAM, and executed by one or more processors of the computing system 3300. Although the method 3200 (or a portion thereof) is described with respect to the computing system 3300 shown in FIG. 33, the description is illustrative only and is not intended to be limiting. In some embodiments, the method 3200 or portions thereof may be performed serially or in parallel by multiple computing systems.

A method for determining protospacer sequences and their profiles (or off-target prediction/determination and/or guide design) can be referred to herein as AVOLANCHE. FIG. 13 shows a non-limiting exemplary flowchart of the AVOLANCHE method. A method for determining protospacer sequences and their profiles can be efficient and fast. A method for determining protospacer sequences and their profiles can be comprehensive (or exhaustive). A method for determining protospacer sequences and their profiles can have search comprehensiveness. A method for determining protospacer sequences and their profiles can be a method that is not a brute force method. A method for determining protospacer sequences and their profiles can avoid user error. A method for determining protospacer sequences and their profiles can be easily updated for new or additional nucleic acid guided nuclease (e.g., Cas proteins) and/or new genomes (or genome sequences). A method for determining protospacer sequences and their profiles can be used for both mismatch gap prediction. A method for determining protospacer sequences and their profiles can have a scalable infrastructure. A method for determining protospacer sequences and their profiles can allow for modular extensions and/or allow new features. A method for determining protospacer sequences and their profiles can have one, some, or all of the performance characteristics described herein. A method for determining protospacer sequences and their profiles can have one, some, or all of the features of the present disclosure.

After the method 3200 begins at block 3204, the method 3200 proceeds to block 3208, where the method includes receiving a plurality of protospacer sequences. For example, a computing system (e.g., the computing system 3300) can receive a plurality of protospacer sequences. The number of protospacer sequences can be, for example, 1, 2, 3, 4, 5, 6, 7, 8, 9, 10, 15, 20, 30, 40, 50, 75, 100, 150, 200, 300, 400, 500, 1000, 2500, 5000, 7500, 10000, or more. The plurality of protospacer sequences can comprise some or all possible protospacer sequences in the sequence of interest. A protospacer sequence when present in a guide can be referred to as a spacer sequence (T(s) in the protospacer sequence would be U(s) in the spacer sequence).

The plurality of protospacer sequences can comprise protospacer sequences in a sequence of interest. The plurality of protospacer sequences can comprise all possible protospacer sequences in a sequence of interest. In some embodiments, the sequence of interest can comprise a gene, or a portion thereof. The sequence of interest can comprise an exon, or a portion thereof, of a gene and/or an intron, or a portion thereof, of a gene.

Receiving the plurality of protospacer sequences can comprise: receiving a sequence of interest. Receiving the plurality of protospacer sequences can comprise: determining the plurality of protospacer sequences in the sequence of interest. Receiving the sequence of interest can comprise: receiving the sequence of interest from a user interface (UI) element (e.g., a text field). A UI element can be a window (e.g., a container window, browser window, text terminal, child window, or message window), a menu (e.g., a menu bar, context menu, or menu extra), an icon, or a tab. A UI element can be for input control (e.g., a checkbox, radio button, dropdown list, list box, button, toggle, text field, or date field). A UI element can be navigational (e.g., a breadcrumb, slider, search field, pagination, slider, tag, icon). A UI element can informational (e.g., a tooltip, icon, progress bar, notification, message box, or modal window). A UI element can be a container (e.g., an accordion). Receiving the sequence of interest can comprise: obtaining the sequence of interest from a file (e.g., a file in a storage device, e.g., a file in FASTA format or CSV format) and/or over a network (e.g., LAN, WAN, or Internet).

Determining the plurality of protospacer sequences in the sequence of interest can comprise: determining the plurality of protospacer sequences in the sequence of interest based on the PAM space. Determining the plurality of protospacer sequences in the sequence of interest based on the PAM space can comprise: identifying an on-target PAM sequence in the sequence of interest. Determining the plurality of protospacer sequences in the sequence of interest based on the PAM space can comprise: identifying a protospacer sequence associated with the on-target PAM sequence in the sequence of interest using a protospacer length (e.g., 20 nucleotides in length, or 15, 16, 17, 18, 19, 20, 21, 22, 23, 24, 25, 26, 27, or more, nucleotides in length), a spacing between an on-target PAM sequence and an associated protospacer sequence (e.g., 1, 2, 3, 4, 5, 6, 7, 8, 9, 10, 11, 12, 13, 14, 15, or more, nucleotides in length), and/or a relative positioning (e.g., 3′ or 5′) of an on-target PAM sequence and an associated protospacer sequence in the PAM space.

In some embodiments, the method comprises: receiving a selection of a nucleic acid guided nuclease (or nucleic acid guided endonuclease or RNA-guided DNA endonuclease), or a portion thereof and/or a variant thereof (e.g., a nickase). The method can comprise: obtaining (or selecting or retrieving) the PAM space associated with the nucleic acid guided nuclease. The method can comprise: receiving a selection of a reference sequence (e.g., a reference genome sequence of hg16, hg17, hg18, hg19, hg38, mm10, canFam4, chlSab2, macFas5, rheMac10, or rn6).

The method 3200 proceeds from block 3208 to block 3212, where the method includes generating a plurality of homology strings of a protospacer sequence (or a protospacer sequence of each of the plurality of protospacer sequences). For example, a computing system (e.g., the computing system 3300) can generate, for each of the plurality of protospacer sequences, a plurality of homology strings of the protospacer sequence. The number of homology strings (of a protospacer sequence) can be, for example, 30, 40, 50, 60, 70, 80, 90, 100, 150, 200, 300, 400, 500, 750, 1000, 2500, 5000, 7500, 10000, or more. See FIG. 4 for an illustration.

Each of the plurality of homology strings (e.g., 30, 40, 50, 60, 70, 80, 90, 100, 150, 200, 300, 400, 500, 750, 1000 or more) of a protospacer sequence can comprise one or more mismatches (mm) (or zero, one, or more mismatches) relative to the protospacer sequence and/or one or more indels (or zero, one, or more indels) relative to the protospacer sequence. An indel can be referred to as a gap. An indel can be an insertion. An indel can be a deletion. The maximum number of mismatches can vary, such as 0, 1, 2, 3, 4, or 5 mismatches. The maximum number of indels can vary, such as 0, 1, 2, 3, 4, or 5 indels. In some embodiments, the maximum number of mismatches can be 5 when there is no indel. The maximum number of mismatches can be 2 when there is 1 indel (or at most 1 indel). The maximum number of mismatches can be 0 when there are 2 indels (or at most 2 indels). A homology string can be of a homology string type. A homology string type can comprise a combination of a number of mismatches and a number of indels, NmmXgap, where N can be for example 0, 1, 2, 3, 4, or 5, and X can be for example 0, 1, or 2, such as 0mm0gap, 1mm0gap, 2mm0gap, 3mm0gap, 4mm0gap, 5mm0gap, 0mm1gap, 1mm1gap, 2mm1gap, or 0mm2gap. In some embodiments, homology strings of the plurality of homology strings of a protospacer sequence with one mismatch, relative to the protospacer sequence, comprise all possible sequences with one mismatch at each position of the protospacer sequence. Homology strings of the plurality of homology strings of a protospacer sequence with two mismatches, relative to the protospacer sequence, can comprise all possible sequences with two mismatches relative to the protospacer sequence. Homology strings of the plurality of homology strings of a protospacer sequence with three mismatches, relative to the protospacer sequence, can comprise all possible sequences with three mismatches relative to the protospacer sequence. Homology strings of the plurality of homology strings of a protospacer sequence with four mismatches, relative to the protospacer sequence, can comprise all possible sequences with four mismatches relative to the protospacer sequence. Homology strings of the plurality of homology strings of a protospacer sequence with five mismatches, relative to the protospacer sequence, can comprise all possible sequences with five mismatches relative to the protospacer sequence. Homology strings of the plurality of homology strings of a protospacer sequence with one indel relative to the protospacer sequence can comprise all sequences with one indel at each position of the protospacer sequence. Homology strings of the plurality of homology strings of a protospacer sequence with two indels relative to the protospacer sequence can comprise all sequences with two indel relative to the protospacer sequence.

In some embodiments, the plurality of homology strings of a protospacer sequence comprises all (comprehensive or exhaustive) homology strings of the protospacer sequence of each of one or more homology string types, optionally wherein homology string type comprises a combination of a number of mismatches (e.g., 0, 1, 2, 3, 4, 5, 6, 7, 8, 9, or 10 mismatches) and a number of indels (e.g., 0, 1, 2, 3, 4, or 5 indels). In some embodiments, the plurality of homology strings of a protospacer sequence comprises the protospacer sequence. Alternatively, the plurality of homology strings of a protospacer sequence does not comprise the protospacer sequence.

The method 3200 proceeds from block 3212 to block 3216, where the method includes mapping (or aligning) each of the plurality of homology strings (or each of homology strings of the plurality of homology strings) to a reference sequence to determine a match (or at least one match, or one or more matches) of the homology string in the reference sequence. For example, a computing system (e.g., the computing system 3300) can maps (or aligns) each of the plurality of homology strings to a reference sequence to determine a match (or at least one match, or one or more matches) of the homology string in the reference sequence. The number of match(es) can be, for example, 1, 2, 3, 4, 5, 10, 15, 20, 30, 40, 50, 100, or more matches. A match can be a perfect match (have zero mismatch) to (a subsequence of) the reference sequence. A match can have a perfect alignment to (a subsequence of) the reference sequence.

A match of a homology string of a protospacer sequence can comprise a perfect alignment (e.g., 0 mismatch) of the homology string to a position of the reference sequence. A corresponding off-target site of the protospacer sequence can comprise an alignment of the off-target site to the position of the reference sequence that is not a perfect alignment. For example, a protospacer sequence can be ATGCATGCATGCATGCATGC (SEQ ID NO: 1) (no associated PAM sequence shown). Homology strings of this protospacer sequence with 1 mismatch at position 9 and no gap can be ATGCATGCTTGCATGCATGC (SEQ ID NO: 2), ATGCATGCGTGCATGCATGC (SEQ ID NO: 3), and ATGCATGCCTGCATGCATGC (SEQ ID NO: 4). A match of the homology string ATGCATGCTTGCATGCATGC (SEQ ID NO: 2) in a reference sequence can be ATGCATGCTTGCATGCATGC (SEQ ID NO: 2), which is an off-target site of the protospacer sequence ATGCATGCATGCATGCATGC (SEQ ID NO: 1) due to the difference of 1 mismatch between the protospacer sequence ATGCATGCATGCATGCATGC (SEQ ID NO: 1) and the homology string ATGCATGCTTGCATGCATGC (SEQ ID NO: 2).

For example, a protospacer sequence can be ATGCATGCATGCATGCATGC (SEQ ID NO: 1) (no associated PAM sequence shown). Homology strings of this protospacer sequence with 0 mismatch and 1 insertion at position 9 can be ATGCATGCAATGCATGCATGC (SEQ ID NO: 5), ATGCATGCTATGCATGCATGC (SEQ ID NO: 6), ATGCATGCGATGCATGCATGC (SEQ ID NO: 7), and ATGCATGCCATGCATGCATGC (SEQ ID NO: 8). A match of the homology string ATGCATGCAATGCATGCATGC (SEQ ID NO: 5) in a reference sequence can be ATGCATGCAATGCATGCATGC (SEQ ID NO: 5), which is an off-target site of the protospacer sequence ATGCATGCATGCATGCATGC (SEQ ID NO: 1) due to the difference of 1 insertion between the protospacer sequence ATGCATGCATGCATGCATGC (SEQ ID NO: 1) and the homology string ATGCATGCAATGCATGCATGC (SEQ ID NO: 5).

Mapping (or aligning) each of the plurality of homology strings to a reference sequence to determine a match (or at least one match, or one or more matches) of the homology string in the reference sequence can be performed using an alignment method such as Burrows-Wheeler Aligner (BWA), ISAAC, BarraCUDA, BFAST, BLASTN, BLAT, Bowtie, CASHX, Cloudburst, CUDA-EC, CUSHAW, CUSHAW2, CUSHAW2-GPU, drFAST, ELAND, ERNE, GNUMAP, GEM, GensearchNGS, GMAP and GSNAP, Geneious Assembler, LAST, MAQ, mrFAST and mrsFAST, MOM, MOSAIK, MPscan, Novoaligh & NovoalignCS, NextGENe, Omixon, PALMapper, Partek, PASS, PerM, PRIMEX, QPalma, RazerS, REAL, cREAL, RMAP, rNA, RT Investigator, Segemehl, SeqMap, Shrec, SHRIMP, SLIDER, SOAP, SOAP2, SOAP3 and SOAP3-dp, SOCS, SSAHA and SSAHA2, Stampy, STORM, Subread and Subjunc, Taipan, UGENE, VelociMapper, XpressAlign, and ZOOM.

The method 3200 proceeds from block 3216 to block 3220, where the method includes filtering (or removing) one or more of the matches of homology strings of the plurality of homology strings of the protospacer sequence to determine one or more off-target sites of the protospacer sequence. For example, a computing system (e.g., the computing system 3300) can filter (or remove) one or more of the matches of homology strings of the plurality of homology strings of the protospacer sequence to determine one or more off-target sites of the protospacer sequence. Filtering (or removing) one or more of the matches of homology strings of the plurality of homology strings of the protospacer sequence to determine one or more off-target sites of the protospacer sequence can be based on a protospacer adjacent motif (PAM) space. The number of off-target sites can be, for example, 100, 1000, 2500, 5000, 7500, 10000, 25000, 50000, 75000, 100000, 250000, 500000, 750000, 1000000, 2500000, 5000000, 7500000, 10000000, or more.

Filtering one or more of the matches of the homology strings can comprise: removing from the matches of the homology strings of the plurality of homology string one or more of the matches of the homology strings. The one or more off-target sites of the protospacer sequence can comprise the remaining matches of the plurality of homology strings. The remaining matches of the plurality of homology strings can be the one or more off-target sites. Filtering one or more of the matches of the homology strings can comprise: filtering a match of a homology string, based on an absence of a PAM sequence (e.g., an on-target PAM sequence) being associated with the match in the reference sequence (e.g., the match does not have an associated PAM sequence in the genome), to determine one or more off-target sites of the protospacer sequence. Filtering one or more of the matches of the homology strings can comprise: filtering a match of a homology string, based on an absence of any on-target PAM sequence and/or any off-target PAM sequence being associated with the match in the reference sequence, to determine one or more off-target sites of the protospacer sequence. The one or more off-target sites of the protospacer sequence can be comprehensive or exhaustive, such as 100%, of the off-target sites of the protospacer sequence. The one or more off-target sites can comprise at least 99% (sor 95%, 96%, 97%, 98%, 99%, 99.9%, 99.99%, or more) of all possible off-target sites of the protospacer sequence.

The PAM space can comprise a PAM sequence. The PAM sequence can be 2, 3, 4, 5, 6, or more nucleotides in length. The PAM space can comprise an on-target PAM sequence (e.g., NGG for SpCas9). Alternatively or additionally, the PAM space can comprise one or more off-target PAM sequences (e.g., NAG, NGA, NAA, NCG, NGC, NTG, and NGT for SpCas9). Alternatively or additionally, the PAM space can comprise a spacing (e.g., 1, 2, 3, 4, 5, 6, 7, 8, 9, or 10 nucleotides) between an PAM sequence and an associated protospacer sequence. Alternatively or additionally, the PAM space can comprise a spacing (e.g., 1, 2, 3, 4, 5, 6, 7, 8, 9, or 10 nucleotides) between an PAM sequence and a cleavage site in an associated protospacer sequence. Alternatively or additionally, the PAM space can comprise a relative positioning (e.g., 3′ or 5′) of an on-target PAM sequence and an associated protospacer sequence. In some embodiments, each of the plurality of protospacer sequences is associated with a PAM sequence (e.g., an on-target PAM sequence) in the reference sequence.

In some embodiments, a nucleic acid guided nuclease (or nucleic acid guided endonuclease or RNA-guided DNA endonuclease), or a portion thereof and/or a variant thereof (e.g., a nickase Cas9 (nCas9)), is associated with the PAM space. The PAM space can be determined based on the specific nucleic acid guided nuclease, which can be selected. The nucleic acid guided nuclease can be associated with a protospacer length (e.g., 20 nucleotides in length, or 15, 16, 17, 18, 19, 20, 21, 22, 23, 24, 25, 27, 27, or more, nucleotides in length). The nucleic acid guided nuclease can be a CRISPR-associated (Cas) nuclease of a species. The nucleic acid guided nuclease can be S. pyogenes Cas9 (SpCas9), S. aureus Cas9 (SaCas9), or S. lugdunensis Cas9 (slCas9). The nucleic acid guided nuclease can be a Class 1 Cas or Class 2 Cas. The nucleic acid guided nuclease can be a Cas of type I, II, III, IV, V, or VI. The nucleic acid guided nuclease can be Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, Cas9, Csn2, Cas4, Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5), C2c4, C2c8, C2c9, Cas13, Cas13a (C2c2), Cas13b, Cas13c, Cas13d, or Cas13x.1.

The method 3200 proceeds from block 3220 to block 3224, where the method includes determining a profile of the protospacer sequence (or a profile of each of one or more protospacer sequences of the plurality of protospacer sequences, or a profile of each of the plurality of protospacer sequences) using the off-target sites of the protospacer sequence. For example, a computing system (e.g., the computing system 3300) can determine a profile of the protospacer sequence (or a profile of each of the plurality of protospacer sequences, a profile of each of one or more protospacer sequences of the plurality of protospacer sequences) using the off-target sites of the protospacer sequence.

The profile of a protospacer sequence can comprise a protospacer sequence score of the protospacer sequence. Determining the profile of the protospacer sequence can comprise: determining a protospacer sequence score of the protospacer sequence using the off-target sites of the protospacer sequence.

The profile of a protospacer sequence can comprise an off-target profile of the protospacer sequence. The profile of a protospacer sequence can comprise a summary of the off-target sites of the protospacer sequence. The summary of the off-target sties of the protospacer sequence can comprise a number of one or more matches of the protospacer sequence in the reference sequence. The summary of the off-target sties of the protospacer sequence can comprise a number of off-target sites of the protospacer sequence for each of one or more homology string types.

Determining the protospacer sequence score of the protospacer sequence comprises: determining an off-target site score for each of the one or more off-target sites of the protospacer sequence. Determining the protospacer sequence score of the protospacer sequence can comprise: determining the protospacer sequence score of the protospacer sequence using the off-target site scores of the one or more off-target sites of the protospacer sequence.

The protospacer sequence score can be based on a number of the off-target sites. The protospacer sequence score can be based on the distribution of mismatches of the off-target sites. The protospacer sequence score can be based on the distance of an off-target site to the closest annotated exon. The protospacer sequence score can reflect a strength of interaction between a guide comprising the protospacer sequence (T(s) in the protospacer sequence would be U(s) in the corresponding spacer sequence of the guide) and a target of the guide. The protospacer sequence score can comprise an off-target score, a CCTop score and/or a CFD score.

LCR. In some embodiments, the method comprises: filtering the one or more off-target sites of the protospacer sequence using low complexity region (LCR) filtering to generated one or more filtered off-target sites. LCR filtering removes any off-target sites that overlap pre-identified LCR regions. So, with LCR filtering, there will be fewer or the same number of off-target sites compared to off-target sites not LCR filtered. This is because there may be no off-target site overlapping LCRs in some instances, and in other instances, there may be 1 or more off-target sites overlapping LCRs. Determining the protospacer sequence score of the protospacer sequence can comprise: determining the protospacer sequence score of the protospacer sequence using the filtered off-target sites of the protospacer sequence. Determining the profile of the protospacer sequence can comprise: determining the profile of the protospacer sequence using the filtered off-target sites of the protospacer sequence.

Consolidation. In some embodiments, there is no consolidation of overlapping off-targets sites. In some embodiments, the method comprises: consolidating two of the off-target sites of a protospacer sequence that overlap to generate consolidated off-target sites of the protospacer sequence. The method can comprises: consolidating overlapping off-target sites of the off-target sites of a protospacer sequence to generate consolidated off-target sites of the protospacer sequence. Consolidation can be based on 1 or more of the following criteria:
Consolidate off-target sites with a certain threshold of overlap
Consolidate off-target sites with the same start or end coordinate
Consolidate off-target sites with the same PAM location and the same start or end coordinate
Consolidate on PAM coordinates
Consolidate sites with same cut and start coordinates
Consolidate sites with the same start and end coordinates
Consolidate sites with the same PAM coordinates, reporting the alignment that's most likely to cut
Consolidate with a hierarchical rule-based system of homology at same PAM coordinates, and then by alignment that's most likely to cut, e.g., 1 mm sites take priority over gap sites, etc.
Consolidate proximal sites with a certain threshold of overlap

Determining the protospacer sequence score comprises: determining a protospacer sequence score of each of the plurality of protospacer sequences based on the consolidated off-target sites of the protospacer sequence.

### Output

In some embodiments, the method comprises: outputting the protospacer sequence of each of one or more protospacer sequences (or each protospacer sequence) of the plurality of protospacer sequences and/or the profile of the protospacer sequence. In some embodiments, the method comprises: ranking and/or sorting the plurality of protospacer sequences based on the protospacer sequence scores and/or the profiles. Outputting each of the plurality of protospacer sequences and the profile of the protospacer sequence can comprise: outputting each of the plurality of protospacer sequences and the profile of the protospacer sequence comprises based on the ranking and/or sorting.

Outputting each of the one or more protospacer sequences and the profile of the protospacer sequence comprises: outputting the profile of the protospacer sequence of each of one or more protospacer sequences and the profile of the protospacer sequence to one or more files. Outputting each of the one or more protospacer sequences and the profile of the protospacer sequence comprises: generating a report comprising the profile of the protospacer sequence of each of the one or more protospacer sequences and the profile of the protospacer sequence. Outputting each of the one or more protospacer sequences and the profile of the protospacer sequence comprises: generating a user interface (UI) comprises one or more UI elements representing the profile of the protospacer sequence of each of the one or more protospacer sequences and the profile of the protospacer sequence. A UI element can be a window (e.g., a container window, browser window, text terminal, child window, or message window), a menu (e.g., a menu bar, context menu, or menu extra), an icon, or a tab. A UI element can be for input control (e.g., a checkbox, radio button, dropdown list, list box, button, toggle, text field, or date field). A UI element can be navigational (e.g., a breadcrumb, slider, search field, pagination, slider, tag, icon). A UI element can informational (e.g., a tooltip, icon, progress bar, notification, message box, or modal window). A UI element can be a container (e.g., an accordion).

### Guide and Editing

In some embodiments, the method can comprise: obtaining a guide comprising a protospacer sequence (T(s) in the protospacer sequence would be U(s) in the corresponding spacer sequence in the guide) of the plurality of protospacer sequences. The guide can be selected based on the profiles of protospacer sequences of the plurality of protospacer sequences (or based on the profile of each of the plurality of protospacer sequences). The method can comprise: selecting the protospacer sequence based on the profiles of one or more protospacer sequences of the plurality of protospacer sequences. The method can comprise: selecting the protospacer sequence based on the profile of each of the plurality of protospacer sequences.

The protospacer sequence selected (or the protospacer sequence of the guide) can have the best profile among profiles of protospacer sequences of the plurality of protospacer sequences (or among the profile of each of the plurality of protospacer sequences). For example, the protospacer sequence selected (or the protospacer sequence of the guide) can have the best protospacer sequence score (e.g., the biggest). For example, the protospacer sequence selected (or the protospacer sequence of the guide) can be the protospacer sequence with fewest predicted off-target sites and/or least impactful off-target sites.

Obtaining the guide can comprise: designing the guide. The guide can comprise a guide ribonucleic acid (RNA). The guide can comprise a single guide RNA (sgRNA). The sgRNA can comprise a prime editing guide RNA (pegRNA).

In some embodiments, the method comprises: editing a sequence in a nucleic acid (e.g., deoxyribonucleic acid (DNA)) using the guide and a nucleic acid guided nuclease (or nucleic acid guided endonuclease or RNA-guided DNA endonuclease), or a portion thereof and/or a variant thereof (e.g., a nickase Cas9 (nCas9)). The editing can be base editing or prime editing. The nucleic acid can be in a cell. The cell can be in a subject, e.g., a mammal, such as a human. The nucleic acid guided nuclease can be a CRISPR-associated (Cas) nuclease of a species. The nucleic acid guided nuclease can be S. pyogenes Cas9 (SpCas9), S. aureus Cas9 (SaCas9), or S. lugdunensis Cas9 (slCas9). The nucleic acid guided nuclease can be a Class 1 Cas or Class 2 Cas. The nucleic acid guided nuclease can be a Cas of type I, II, III, IV, V, or VI. The nucleic acid guided nuclease can be Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, Cas9, Csn2, Cas4, Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5), C2c4, C2c8, C2c9, Cas13, Cas13a (C2c2), Cas13b, Cas13c, Cas13d, or Cas13x.1.

In some embodiments, the method comprises: determining an empirical profile of the guide. The empirical profile can comprise, for example, editing efficiency, or off-target profile.

The method 3200 ends at block 3228.

### Execution Environment

FIG. 33 depicts a general architecture of an example computing device 3300 that can be used in some embodiments to execute the processes and implement the features described herein. The general architecture of the computing device 3300 depicted in FIG. 33 includes an arrangement of computer hardware and software components. The computing device 3300 may include many more (or fewer) elements than those shown in FIG. 33. It is not necessary, however, that all of these generally conventional elements be shown in order to provide an enabling disclosure. As illustrated, the computing device 3300 includes a processing unit 3310, a network interface 3320, a computer readable medium drive 3330, an input/output device interface 3340, a display 3350, and an input device 3360, all of which may communicate with one another by way of a communication bus. The network interface 3320 may provide connectivity to one or more networks or computing systems. The processing unit 3310 may thus receive information and instructions from other computing systems or services via a network. The processing unit 3310 may also communicate to and from memory 3370 and further provide output information for an optional display 3350 via the input/output device interface 3340. The input/output device interface 3340 may also accept input from the optional input device 3360, such as a keyboard, mouse, digital pen, microphone, touch screen, gesture recognition system, voice recognition system, gamepad, accelerometer, gyroscope, or other input device.

The memory 3370 may contain computer program instructions (grouped as modules or components in some embodiments) that the processing unit 3310 executes in order to implement one or more embodiments. The memory 3370 generally includes RAM, ROM and/or other persistent, auxiliary or non-transitory computer-readable media. The memory 3370 may store an operating system 3372 that provides computer program instructions for use by the processing unit 3310 in the general administration and operation of the computing device 3300. The memory 3370 may further include computer program instructions and other information for implementing aspects of the present disclosure.

For example, in one embodiment, the memory 3370 includes a guide module 3374 for guide design and/or off-target searches. In addition, memory 3370 may include or communicate with the data store 3390 and/or one or more other data stores that store the input data, intermediate results, and/or final results of guide design and/or off-target searches described herein.

### Additional Considerations

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C can include a first processor configured to carry out recitation A and working in conjunction with a second processor configured to carry out recitations B and C. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

What is claimed is:

1. A system for determining protospacer sequences in a sequence of interest comprising:
non-transitory memory configured to store executable instructions; and
a hardware processor in communication with the non-transitory memory, the hardware processor programmed by the executable instructions to perform:
receiving a sequence of interest;
determining a plurality of protospacer sequences in the sequence of interest;
generating a plurality of homology strings of each of the plurality of protospacer sequences;
mapping each of the plurality of homology strings to a reference sequence to determine a match of the homology string in the reference sequence;
filtering one or more of the matches of each of one or more homology strings of the plurality of homology strings, based on a protospacer adjacent motif (PAM) space, to determine one or more off-target sites of the protospacer sequence;
determining a protospacer sequence score of each of the plurality of protospacer sequences based on the off-target sites of the protospacer sequence;
determining a profile, of each of the plurality of protospacer sequences, comprising the protospacer sequence score of the protospacer sequence and based on the off-target sites of the protospacer sequence; and
outputting each of the plurality of protospacer sequences and the profile of the protospacer sequence.

2. A system for determining protospacer sequences in a sequence of interest comprising:
non-transitory memory configured to store executable instructions; and
a hardware processor in communication with the non-transitory memory, the hardware processor programmed by the executable instructions to perform:
receiving a plurality of protospacer sequences;
generating a plurality of homology strings of each of the plurality of protospacer sequences;
mapping each of the plurality of homology strings to a reference sequence to determine a match of the homology string in the reference sequence;
filtering one or more of the matches of each of one or more homology strings of the plurality of homology strings, based on a protospacer adjacent motif (PAM) space, to determine one or more off-target sites of the protospacer sequence;
determining a protospacer sequence score of each of the plurality of protospacer sequences based on the off-target sites of the protospacer sequence;
outputting each of the plurality of protospacer sequences and the protospacer sequence score of the protospacer sequence.

3. The system of claim 2, wherein the hardware processor is programmed by the executable instructions to perform: determining a profile, of each of the plurality of protospacer sequences, comprising the protospacer sequence score of the protospacer sequence and/or based on the off-target sites of the protospacer sequence, and wherein outputting each of the plurality of protospacer sequences and the protospacer sequence score of the protospacer sequence comprises: outputting each of the plurality of protospacer sequences and the profile of the protospacer sequence.

4. A system for determining profiles of protospacer sequences comprising:
non-transitory memory configured to store executable instructions; and
a hardware processor in communication with the non-transitory memory, the hardware processor programmed by the executable instructions to perform:
receiving a plurality of protospacer sequences;
for each of the plurality of protospacer sequences:
generating a plurality of homology strings of the protospacer sequence;
mapping each of the plurality of homology strings to a reference sequence to determine a match of the homology string in the reference sequence;
filtering one or more of the matches of homology strings of the plurality of homology strings, based on a protospacer adjacent motif (PAM) space, to determine one or more off-target sites of the protospacer sequence; and
determining a profile of the protospacer sequence using the off-target sites of the protospacer sequence.

5. The system of claim 4, wherein the profile of a protospacer sequence comprises a protospacer sequence score of the protospacer sequence.

6. The system of any one of claims 4-5, wherein the hardware processor is programmed by the executable instructions to perform: outputting the profile of the protospacer sequence of each of one or more of the plurality of protospacer sequences.

7. The system of any one of claims 2-6, wherein the plurality of protospacer sequences comprises protospacer sequences in the sequence of interest.

8. The system of any one of claims 2-7, wherein receiving the plurality of protospacer sequences comprises:
receiving a sequence of interest; and
determining the plurality of protospacer sequences in the sequence of interest.

9. The system of any one of claims 1-8, wherein receiving the sequence of interest comprises: receiving the sequence of interest from a user interface (UI) element.

10. The system of any one of claims 1-9, wherein receiving the sequence of interest comprises: obtaining the sequence of interest from a file or over a network.

11. The system of any one of claims 1-10, wherein the sequence of interest comprises a gene, or a portion thereof, optionally wherein the sequence of interest comprises an exon, or a portion thereof, of a gene and/or an intron, or a portion thereof, of a gene.

12. The system of any one of claims 1-11, wherein the PAM space comprises an on-target PAM sequence, one or more off-target PAM sequences, a spacing between an on-target PAM sequence and an associated protospacer sequence, a spacing between an on-target PAM sequence and a cleavage site in an associated protospacer sequence, and/or a relative positioning of an on-target PAM sequence and an associated protospacer sequence.

13. The system of any one of claims 1-12, wherein each of the plurality of protospacer sequences is associated with a PAM sequence in the reference sequence.

14. The system of any one of claims 1-13,
wherein determining the plurality of protospacer sequences in the sequence of interest comprises: determining the plurality of protospacer sequences in the sequence of interest based on the PAM space,
optionally wherein determining the plurality of protospacer sequences in the sequence of interest based on the PAM space comprises:
identifying an on-target PAM sequence in the sequence of interest;
identifying a protospacer sequence associated with the on-target PAM sequence in the sequence of interest using a protospacer length, a spacing between an on-target PAM sequence and an associated protospacer sequence, and/or a relative positioning of an on-target PAM sequence and an associated protospacer sequence in the PAM space.

15. The system of any one of claims 1-14, wherein a nucleic acid guided nuclease, or a portion thereof and/or a variant thereof, is associated with the PAM space and a protospacer length, optionally wherein the nucleic acid guided nuclease is a CRISPR-associated (Cas) nuclease of a species, and optionally wherein nucleic acid guided nuclease is S. pyogenes Cas9, S. aureus Cas9, or S. lugdunensis Cas9,

16. The system of any one of claims 1-15, wherein the hardware processor is programmed by the executable instructions to perform:
receiving a selection of a nucleic acid guided nuclease, or a portion thereof and/or a variant thereof;
obtaining the PAM space associated with the nucleic acid guided nuclease; and/or
receiving a selection of a reference sequence.

17. The system of any one of claims 1-16, wherein each of the plurality of homology strings of a protospacer sequence comprises one or more mismatches relative to the protospacer sequence and/or one or more indels relative to the protospacer sequence.

18. The system of claim 17, wherein homology strings of the plurality of homology strings of a protospacer sequence with one mismatch, relative to the protospacer sequence, comprise all possible sequences with one mismatch at each position of the protospacer sequence, wherein homology strings of the plurality of homology strings of a protospacer sequence with two mismatches, relative to the protospacer sequence, comprise all possible sequences with two mismatches relative to the protospacer sequence, wherein homology strings of the plurality of homology strings of a protospacer sequence with one indel relative to the protospacer sequence comprise all sequences with one indel at each position of the protospacer sequence, and/or wherein homology strings of the plurality of homology strings of a protospacer sequence with two indels relative to the protospacer sequence comprise all sequences with two indel relative to the protospacer sequence.

19. The system of any one of claims 1-18, wherein the plurality of homology strings of a protospacer sequence comprises all homology strings of the protospacer sequence of each of one or more homology string types, optionally wherein homology string type comprises a combination of a number of mismatches and a number of indels.

20. The system of any one of claims 1-19, wherein the plurality of homology strings of a protospacer sequence comprises the protospacer sequence, or wherein the plurality of homology strings of a protospacer sequence does not comprise the protospacer sequence.

21. The system of any one of claims 1-20, wherein a match of a homology string of a protospacer sequence comprises a perfect alignment of the homology string to a position of the reference sequence, and wherein a corresponding off-target site of the protospacer sequence comprises an alignment of the off-target site to the position of the reference sequence that is not a perfect alignment.

22. The system of any one of claims 1-21, wherein filtering one or more of the matches of each of the one or more homology strings comprises: removing from the matches of each of the one or more homology strings one or more of the matches of the homology string with the one or more off-target sites of the protospacer sequence comprise the remaining matches of the homology string.

23. The system of any one of claims 1-22, wherein filtering one or more of the matches of the one or more homology strings comprises: filtering a match of a homology string, based on an absence of a PAM sequence being associated with the match in the reference sequence, to determine one or more off-target sites of the protospacer sequence.

24. The system of any one of claims 1-23, wherein the one or more off-target sites of the protospacer sequence are comprehensive of the off-target sites of the protospacer sequence, and/or wherein the one or more off-target sites comprise at least 99% of all possible off-target sites of the protospacer sequence.

25. The system of any one of claims 1-24,
wherein the hardware processor is programmed by the executable instructions to perform: filtering the one or more off-target sites of the protospacer sequence using low complexity region filtering to generated one or more filtered off-target sites,
wherein determining the protospacer sequence score of each of the plurality of protospacer sequences comprises determining the protospacer sequence score of each of the plurality of protospacer sequences based on the filtered off-target sites of the protospacer sequence, and
wherein determining the profile of each of the plurality of protospacer sequences comprises: determining the profile, of each of the plurality of protospacer sequences, comprising the protospacer sequence score of the protospacer sequence and based on the filtered off-target sites of the protospacer sequence.

26. The system of any one of claims 1-25, wherein determining the protospacer sequence score of each of the plurality of protospacer sequences comprises:
determining an off-target site score for each of the one or more off-target sites of the protospacer sequence; and
determining a protospacer sequence score of each of the plurality of protospacer sequences using the off-target site scores of the one or more off-target sites of the protospacer sequence.

27. The system of any one of claims 1-26,
wherein the protospacer sequence score is based on a number of the off-target sites, the distribution of mismatches of the off-target sites, and/or the distance of an off-target site to the closest annotated exon,
wherein the protospacer sequence score reflects a strength of interaction between a guide comprising the protospacer sequence and a target of the guide, and/or
wherein the protospacer sequence score comprises an off-target score, a CCTop score and/or a CFD score.

28. The system of any one of claims 1-27, wherein the hardware processor programmed by the executable instructions to perform: consolidating two of the off-target sites of a protospacer sequence that overlap to generate consolidated off-target sites of the protospacer sequence, and/or consolidating overlapping off-target sites of the off-target sites of a protospacer sequence to generate consolidated off-target sites of the protospacer sequence.

29. The system of claim 28, wherein determining the protospacer sequence score comprises: determining a protospacer sequence score of each of the plurality of protospacer sequences based on the consolidated off-target sites of the protospacer sequence

30. The system of any one of claims 1-29, wherein the profile of a protospacer sequence comprises an off-target profile of the protospacer sequence

31. The system of any one of claims 1-30, wherein the profile of a protospacer sequence comprises a summary of the off-target sites of the protospacer sequence, optionally wherein the summary of the off-target sties of the protospacer sequence comprises a number of one or more matches of the protospacer sequence in the reference sequence and/or a number of off-target sites of the protospacer sequence for each of one or more homology string types.

32. The system of any one of claims 1-31, wherein the hardware processor programmed by the executable instructions to perform: ranking and/or sorting the plurality of protospacer sequences based on the protospacer sequence scores and/or the profiles, and wherein outputting each of the plurality of protospacer sequences and the profile of the protospacer sequence comprises: outputting each of the plurality of protospacer sequences and the profile of the protospacer sequence comprises based on the ranking and/or sorting.

33. The system of any one of claims 1-32, wherein outputting each of the protospacer sequences and the profile of the protospacer sequence comprises: outputting each of the plurality of protospacer sequences and the profile of the protospacer sequence to one or more files.

34. The system of any one of claims 1-33, wherein outputting each of the protospacer sequences and the profile of the protospacer sequence comprises: generating a user interface (UI) comprises one or more UI elements representing each of the plurality of protospacer sequences and the profile of the protospacer sequence.

35. A method for determining a profile of a protospacer sequence comprising:
under control of a hardware processor:
receiving a sequence of interest;
determining a protospacer sequence in the sequence of interest;
generating homology strings of the protospacer sequence;
mapping the homology strings to a reference sequence to determine matches of the homology strings in the reference sequence;
filtering one or more of the matches of the homology strings, based on a protospacer adjacent motif (PAM) space, to determine one or more off-target sites of the protospacer sequence; and
determining a profile of the protospacer sequence using the off-target sites of the protospacer sequence.

36. A method for determining a profile of a protospacer sequence comprising:
receiving a protospacer sequence in a sequence of interest;
generating a plurality of homology strings of the protospacer sequence;
mapping each of one or more of the plurality of homology strings to a reference sequence to determine a match of the homology string in the reference sequence;
filtering one or more of the matches of homology strings of the plurality of homology strings, based on a protospacer adjacent motif (PAM) space, to determine one or more off-target sites of the protospacer sequence; and
determining a profile of the protospacer sequence using the off-target sites of the protospacer sequence.

37. The method of any one of claims 35-36, comprising: outputting the protospacer sequence and the profile of the protospacer sequence.

38. A method for editing a sequence comprising:
obtaining a guide comprising a protospacer sequence of a sequence of interest, wherein the protospacer sequence is selected from a plurality of protospacer sequences of the sequence of interest by:
for each of the plurality of protospacer sequences of the sequence of interest:
generating a plurality of homology strings of the protospacer sequence;
mapping each of the plurality of homology strings to a reference sequence to determine a match of the homology string in the reference sequence;
filtering one or more of the matches of homology strings of the plurality of homology strings, based on a protospacer adjacent motif (PAM) space, to determine one or more off-target sites of the protospacer sequence;
determining a profile of the protospacer sequence using the off-target sites of the protospacer sequence; and
selecting the protospacer sequence from the plurality of protospacer sequences of the sequence of interest based on the profile of each of one or more of the plurality of protospacer sequences; and
editing a sequence in a nucleic acid using the guide and a nucleic acid guided nuclease, or a portion thereof and/or a variant thereof.

39. A method for generating a guide for editing a sequence comprising:
receiving a plurality of protospacer sequences;
for each of the plurality of protospacer sequences:
generating a plurality of homology strings of the protospacer sequence;
mapping each of the plurality of homology strings to a reference sequence to determine a match of the homology string in the reference sequence;
filtering one or more of the matches of homology strings of the plurality of homology strings, based on a protospacer adjacent motif (PAM) space, to determine one or more off-target sites of the protospacer sequence; and
determining a profile of the protospacer sequence using the off-target sites of the protospacer sequence; and
obtaining a guide comprising a protospacer sequence of the plurality of protospacer sequences.

40. The method of any one of claims 35-39, wherein the protospacer sequence is selected based on the profiles of protospacer sequences of the plurality of protospacer sequences.

41. The method of any one of claims 35-40, comprising: selecting the protospacer sequence based on the profiles of protospacer sequences of the plurality of protospacer sequences.

42. The method of any one of claims 35-41, wherein the protospacer sequence of the guide has the best profile among profiles of protospacer sequences of the plurality of protospacer sequences.

43. The method of any one of claims 35-42, wherein obtaining the guide comprises: designing the guide.

44. The method of any one of claims 35-43, wherein the guide comprises a guide ribonucleic acid (gRNA), optionally wherein the guide comprises a single guide RNA (sgRNA), optionally wherein the sgRNA comprises a prime editing guide RNA (pegRNA).

45. The method of any one of claims 35-44, comprising: editing a sequence in a nucleic acid using the guide and a nucleic acid guided nuclease, or a portion thereof and/or a variant thereof, optionally wherein the editing is base editing or prime editing, optionally wherein the nucleic acid is in a cell, optionally wherein the cell is in a subject, optionally wherein the subject is a mammal, and optionally wherein the mammal is a human.

46. The method of any one of claims 35-45, comprising: determining an empirical profile of the guide.

47. The method of any one of claims 35-46, wherein the profile of a protospacer sequence comprises a protospacer sequence score of the protospacer sequence, and wherein determining the profile of the protospacer sequence comprises: determining a protospacer sequence score of the protospacer sequence using the off-target sites of the protospacer sequence.

48. The method of any one of claims 35-47, comprising: outputting the profile of the protospacer sequence of each of one or more of the plurality of protospacer sequences.

49. The method of any one of claims 35-48, wherein the plurality of protospacer sequences comprises protospacer sequences in a sequence of interest.

50. The method of any one of claims 35-49, wherein receiving the plurality of protospacer sequences comprises:
receiving a sequence of interest; and
determining the plurality of protospacer sequences in the sequence of interest.

51. The method of any one of claims 35-50, wherein receiving the sequence of interest comprises: receiving the sequence of interest from a user interface (UI) element.

52. The method of any one of claims 35-51, wherein receiving the sequence of interest comprises: obtaining the sequence of interest from a file or over a network.

53. The method of any one of claims 35-52, wherein the sequence of interest comprises a gene, or a portion thereof, optionally wherein the sequence of interest comprises an exon, or a portion thereof, of a gene and/or an intron, or a portion thereof, of a gene.

54. The method of any one of claims 35-53, wherein the PAM space comprises an on-target PAM sequence, one or more off-target PAM sequences, a spacing between an on-target PAM sequence and an associated protospacer sequence, a spacing between an on-target PAM sequence and a cleavage site in an associated protospacer sequence, and/or a relative positioning of an on-target PAM sequence and an associated protospacer sequence.

55. The method of any one of claims 35-54, wherein each of the plurality of protospacer sequences is associated with a PAM sequence in the reference sequence.

56. The method of any one of claims 35-55,
wherein determining the plurality of protospacer sequences in the sequence of interest comprises: determining the plurality of protospacer sequences in the sequence of interest based on the PAM space,
optionally wherein determining the plurality of protospacer sequences in the sequence of interest based on the PAM space comprises:
identifying an on-target PAM sequence in the sequence of interest;
identifying a protospacer sequence associated with the on-target PAM sequence in the sequence of interest using a protospacer length, a spacing between an on-target PAM sequence and an associated protospacer sequence, and/or a relative positioning of an on-target PAM sequence and an associated protospacer sequence in the PAM space.

57. The method of any one of claims 35-56, wherein a nucleic acid guided nuclease is associated with the PAM space and a protospacer length, optionally wherein the nucleic acid guided nuclease is a CRISPR-associated (Cas) nuclease of a species, and optionally wherein nucleic acid guided nuclease is S. pyogenes Cas9, S. aureus Cas9, or S. lugdunensis Cas9,

58. The method of any one of claims 35-57, comprising:
receiving a selection of a nucleic acid guided nuclease, or a portion thereof and/or a variant thereof;
obtaining the PAM space associated with the nucleic acid guided nuclease; and/or
receiving a selection of a reference sequence.

59. The method of any one of claims 35-58, wherein each of the plurality of homology strings of a protospacer sequence comprises one or more mismatches relative to the protospacer sequence and/or one or more indels relative to the protospacer sequence.

60. The system of claim 59, wherein homology strings of the plurality of homology strings of a protospacer sequence with one mismatch, relative to the protospacer sequence, comprise all possible sequences with one mismatch at each position of the protospacer sequence, wherein homology strings of the plurality of homology strings of a protospacer sequence with two mismatches, relative to the protospacer sequence, comprise all possible sequences with two mismatches relative to the protospacer sequence, wherein homology strings of the plurality of homology strings of a protospacer sequence with one indel relative to the protospacer sequence comprise all sequences with one indel at each position of the protospacer sequence, and/or wherein homology strings of the plurality of homology strings of a protospacer sequence with two indels relative to the protospacer sequence comprise all sequences with two indels relative to the protospacer sequence.

61. The method of any one of claims 35-60, wherein the plurality of homology strings of a protospacer sequence comprises all homology strings of the protospacer sequence of each of one or more homology string types, optionally wherein homology string type comprises a combination of a number of mismatches and a number of indels.

62. The method of any one of claims 35-61, wherein the plurality of homology strings of a protospacer sequence comprises the protospacer sequence, or wherein the plurality of homology strings of a protospacer sequence does not comprise the protospacer sequence.

63. The method of any one of claims 35-62, wherein a match of a homology string of a protospacer sequence comprises a perfect alignment of the homology string to a position of the reference sequence, and wherein a corresponding off-target site of the protospacer sequence comprises an alignment of the off-target site to the position of the reference sequence that is not a perfect alignment.

64. The method of any one of claims 35-63, wherein filtering one or more of the matches of the homology strings comprises: removing from the matches of the homology strings of the plurality of homology string one or more of the matches of the homology strings with the one or more off-target sites of the protospacer sequence comprise the remaining matches of the plurality of homology strings.

65. The method of any one of claims 35-64, wherein filtering one or more of the matches of the homology strings comprises: filtering a match of a homology string, based on an absence of a PAM sequence being associated with the match in the reference sequence, to determine one or more off-target sites of the protospacer sequence.

66. The method of any one of claims 35-65, wherein the one or more off-target sites of the protospacer sequence are comprehensive of the off-target sites of the protospacer sequence, and/or wherein the one or more off-target sites comprise at least 99% of all possible off-target sites of the protospacer sequence.

67. The method of any one of claims 35-66,
further comprising: filtering the one or more off-target sites of the protospacer sequence using low complexity region filtering to generated one or more filtered off-target sites,
determining the profile of the protospacer sequence comprises: determining the profile of the protospacer sequence using the filtered off-target sites of the protospacer sequence.

68. The method of any one of claims 35-67, wherein determining the protospacer sequence score of the protospacer sequence comprises:
determining an off-target site score for each of the one or more off-target sites of the protospacer sequence; and
determining the protospacer sequence score of the protospacer sequence using the off-target site scores of the one or more off-target sites of the protospacer sequence.

69. The method of any one of claims 35-68,
wherein the protospacer sequence score is based on a number of the off-target sites, the distribution of mismatches of the off-target sites, and/or the distance of an off-target site to the closest annotated exon,
wherein the protospacer sequence score reflects a strength of interaction between a guide comprising the protospacer sequence and a target of the guide, and/or
wherein the protospacer sequence score comprises an off-target score, a CCTop score and/or a CFD score.

70. The method of any one of claims 35-69, comprising: consolidating two of the off-target sites of a protospacer sequence that overlap to generate consolidated off-target sites of the protospacer sequence, and/or consolidating overlapping off-target sites of the off-target sites of a protospacer sequence to generate consolidated off-target sites of the protospacer sequence.

71. The system of claim 70, wherein determining the protospacer sequence score comprises: determining a protospacer sequence score of each of the plurality of protospacer sequences based on the consolidated off-target sites of the protospacer sequence

72. The method of any one of claims 35-71, wherein the profile of a protospacer sequence comprises an off-target profile of the protospacer sequence

73. The method of any one of claims 35-72, wherein the profile of a protospacer sequence comprises a summary of the off-target sites of the protospacer sequence, optionally wherein the summary of the off-target sties of the protospacer sequence comprises a number of one or more matches of the protospacer sequence in the reference sequence and/or a number of off-target sites of the protospacer sequence for each of one or more homology string types.

74. The method of any one of claims 35-73, comprising: ranking and/or sorting the plurality of protospacer sequences based on the protospacer sequence scores and/or the profiles, and wherein outputting each of the plurality of protospacer sequences and the profile of the protospacer sequence comprises: outputting each of the plurality of protospacer sequences and the profile of the protospacer sequence comprises based on the ranking and/or sorting.

75. The method of any one of claims 35-74, wherein outputting each of the protospacer sequences and the profile of the protospacer sequence comprises: outputting the profile of the protospacer sequence of each of one or more of the plurality of protospacer sequences and the profile of the protospacer sequence to one or more files.

76. The method of any one of claims 35-75, wherein outputting each of the protospacer sequences and the profile of the protospacer sequence comprises: generating a user interface (UI) comprises one or more UI elements representing, or a report comprising, the profile of the protospacer sequence of each of one or more of the plurality of protospacer sequences and the profile of the protospacer sequence.


SEQUENCE LISTING (15 sequences)

SEQ ID NO: 1 - DNA, 20 bp, synthetic construct
  source 1..20: mol_type = other DNA
atgcatgcatgcatgcatgc

SEQ ID NO: 2 - DNA, 20 bp, synthetic construct
  source 1..20: mol_type = other DNA
atgcatgcttgcatgcatgc

SEQ ID NO: 3 - DNA, 20 bp, synthetic construct
  source 1..20: mol_type = other DNA
atgcatgcgtgcatgcatgc

SEQ ID NO: 4 - DNA, 20 bp, synthetic construct
  source 1..20: mol_type = other DNA
atgcatgcctgcatgcatgc

SEQ ID NO: 5 - DNA, 21 bp, synthetic construct
  source 1..21: mol_type = other DNA
atgcatgcaatgcatgcatgc

SEQ ID NO: 6 - DNA, 21 bp, synthetic construct
  source 1..21: mol_type = other DNA
atgcatgctatgcatgcatgc

SEQ ID NO: 7 - DNA, 21 bp, synthetic construct
  source 1..21: mol_type = other DNA
atgcatgcgatgcatgcatgc

SEQ ID NO: 8 - DNA, 21 bp, synthetic construct
  source 1..21: mol_type = other DNA
atgcatgccatgcatgcatgc

SEQ ID NO: 9 - DNA, 33 bp, synthetic construct
  source 1..33: mol_type = other DNA
cgacgactgactgactgactgactgggacgacg

SEQ ID NO: 10 - DNA, 20 bp, synthetic construct
  source 1..20: mol_type = other DNA
gactgactgactgactgact

SEQ ID NO: 11 - DNA, 33 bp, synthetic construct
  source 1..33: mol_type = other DNA
cgacgactggctgaatgactgactgggacgacg

SEQ ID NO: 12 - DNA, 20 bp, synthetic construct
  source 1..20: mol_type = other DNA
gactgactgactgactgact

SEQ ID NO: 13 - DNA, 23 bp, synthetic construct
  source 1..23: mol_type = other DNA
gactgactgactgactgactngg

SEQ ID NO: 14 - DNA, 23 bp, synthetic construct
  source 1..23: mol_type = other DNA
gacagactgaatgactgacaacg

SEQ ID NO: 15 - DNA, 22 bp, synthetic construct
  source 1..22: mol_type = other DNA
gacgactgactgactgactacg